SEQUENCE LISTING (303 sequences)

SEQ ID NO: 1 - protein, 13 aa, Artificial
  Synthetic peptide that binds C. difficile
VPAKLLRVIKKIP

SEQ ID NO: 2 - protein, 14 aa, Artificial
  Synthetic peptide that binds C. difficile
VPAKLLRVIDEIPE

SEQ ID NO: 3 - protein, 13 aa, Artificial
  Synthetic peptide that binds C. difficile
VPAKLLRVIDEIP

SEQ ID NO: 4 - protein, 13 aa, Artificial
  Synthetic peptide that binds C. difficile
VPAKLLRVIKKIP

SEQ ID NO: 5 - protein, 13 aa, Artificial
  Synthetic peptide that binds C. difficile
VPAKLLRVIKEIP

SEQ ID NO: 6 - protein, 14 aa, Artificial
  Synthetic peptide that binds C. difficile
VPAKLLRVIKKIPE

SEQ ID NO: 7 - protein, 14 aa, Artificial
  Synthetic peptide that binds C. difficile
EPIEDIVRLLKAPV

SEQ ID NO: 8 - protein, 13 aa, Artificial
  Synthetic peptide that binds C. difficile
PIEDIVRLLKAPV

SEQ ID NO: 9 - protein, 14 aa, Artificial
  Synthetic peptide that binds C. difficile
EPIKKIVRLLKAPV

SEQ ID NO: 10 - protein, 13 aa, Artificial
  Synthetic peptide that binds C. difficile
PIKKIVRLLKAPV

SEQ ID NO: 11 - protein, 14 aa, Artificial
  Synthetic peptide that binds C. difficile
LATKLKYEKEHKKM

SEQ ID NO: 12 - protein, 14 aa, Artificial
  Synthetic peptide that binds C. difficile
LATLKKYLKEHKKM

SEQ ID NO: 13 - protein, 13 aa, Artificial
  Synthetic peptide that binds C. difficile
ATKLKYEKEHKKM

SEQ ID NO: 14 - protein, 13 aa, Artificial
  Synthetic peptide that binds C. difficile
LATKLKYEKEHKK

SEQ ID NO: 15 - protein, 12 aa, Artificial
  Synthetic peptide that binds C. difficile
ATKLKYEKEHKK

SEQ ID NO: 16 - protein, 14 aa, Artificial
  Synthetic peptide that binds C. difficile
MKKHEKEYKLKTAL

SEQ ID NO: 17 - protein, 13 aa, Artificial
  Synthetic peptide that binds C. difficile
MKKHEKEYKLKTA

SEQ ID NO: 18 - protein, 13 aa, Artificial
  Synthetic peptide that binds C. difficile
KKHEKEYKLKTAL

SEQ ID NO: 19 - protein, 12 aa, Artificial
  Synthetic peptide that binds C. difficile
KKHEKEYKLKTA

SEQ ID NO: 20 - protein, 11 aa, Artificial
  Synthetic peptide that binds C. difficile
NILRVLKQVWK

SEQ ID NO: 21 - protein, 10 aa, Artificial
  Synthetic peptide that binds C. difficile
ILRVLKQVWK

SEQ ID NO: 22 - protein, 10 aa, Artificial
  Synthetic peptide that binds C. difficile
NILRVLKQVW

SEQ ID NO: 23 - protein, 9 aa, Artificial
  Synthetic peptide that binds C. difficile
ILRVLKQVW

SEQ ID NO: 24 - protein, 11 aa, Artificial
  Synthetic peptide that binds C. difficile
KWVQKLVRLIN

SEQ ID NO: 25 - protein, 10 aa, Artificial
  Synthetic peptide that binds C. difficile
WVQKLVRLIN

SEQ ID NO: 26 - protein, 10 aa, Artificial
  Synthetic peptide that binds C. difficile
KWVQKLVRLI

SEQ ID NO: 27 - protein, 9 aa, Artificial
  Synthetic peptide that binds C. difficile
WVQKLVRLI

SEQ ID NO: 28 - protein, 12 aa, Artificial
  Synthetic peptide that binds C. difficile
GNFYRLFKDILK

SEQ ID NO: 29 - protein, 11 aa, Artificial
  Synthetic peptide that binds C. difficile
NFYRLFKDILK

SEQ ID NO: 30 - protein, 11 aa, Artificial
  Synthetic peptide that binds C. difficile
GNFYRLFKDIL

SEQ ID NO: 31 - protein, 10 aa, Artificial
  Synthetic peptide that binds C. difficile
NFYRLFKDIL

SEQ ID NO: 32 - protein, 12 aa, Artificial
  Synthetic peptide that binds C. difficile
KLIDKFLRYFNG

SEQ ID NO: 33 - protein, 11 aa, Artificial
  Synthetic peptide that binds C. difficile
LIDKFLRYFNG

SEQ ID NO: 34 - protein, 11 aa, Artificial
  Synthetic peptide that binds C. difficile
KLIDKFLRYFN

SEQ ID NO: 35 - protein, 10 aa, Artificial
  Synthetic peptide that binds C. difficile
LIDKFLRYFN

SEQ ID NO: 36 - protein, 16 aa, Artificial
  Antimicrobial peptide
FIGAIARLLSKIFGKR

SEQ ID NO: 37 - protein, 18 aa, Artificial
  Antimicrobial peptide
GIFSKLAGKKIKNLLISG

SEQ ID NO: 38 - protein, 21 aa, Artificial
  Antimicrobial peptide
GIFSKLAGKKIKNLLISGLKG

SEQ ID NO: 39 - protein, 20 aa, Artificial
  Antimicrobial peptide
GLFSKFVGKGIKNFLIKGVK

SEQ ID NO: 40 - protein, 19 aa, Artificial
  Antimicrobial peptide
KAYSTPRCKGLFRALMCWL

SEQ ID NO: 41 - protein, 19 aa, Artificial
  Antimicrobial peptide
KIFGAIWPLALGALKNLIK

SEQ ID NO: 42 - protein, 15 aa, Artificial
  Antimicrobial peptide
FLKFLKKFFKKLKYY

SEQ ID NO: 43 - protein, 25 aa, Artificial
  Antimicrobial peptide
GWGSFFKKAAHVGKHVGKAALTHYL

SEQ ID NO: 44 - protein, 18 aa, Artificial
  Antimicrobial peptide
RGLRRLGRKIAHGVKKYG

SEQ ID NO: 45 - protein, 29 aa, Artificial
  Antimicrobial peptide
RGLRRLGRKIAHGVKKYGPTVLRIIRIAG

SEQ ID NO: 46 - protein, 18 aa, Artificial
  Antimicrobial peptide
KIAHGVKKYGPTVLRIIR

SEQ ID NO: 47 - protein, 37 aa, Artificial
  Antimicrobial peptide
LLGDFFRKSKEKIGKEFKRIVQRIKDFLRNLVPRTES

SEQ ID NO: 48 - protein, 13 aa, Artificial
  Antimicrobial peptide
FLPLIGRVLSGIL

SEQ ID NO: 49 - protein, 21 aa, Artificial
  Antimicrobial peptide
IGKFLKKAKKFGKAFVKILKK

SEQ ID NO: 50 - protein, 18 aa, Artificial
  Antimicrobial peptide
GKFLKKAKKFGKAFVKIL

SEQ ID NO: 51 - protein, 15 aa, Artificial
  Antimicrobial peptide
WFLKFLKKFFKKLKY

SEQ ID NO: 52 - protein, 14 aa, Artificial
  Antimicrobial peptide
WFLKFLKKFFKKLK

SEQ ID NO: 53 - protein, 17 aa, Artificial
  Antimicrobial peptide
RGLRRLGRKIAHGVKKY

SEQ ID NO: 54 - protein, 13 aa, Artificial
  Antimicrobial peptide
LLGDFFRKSKEKI

SEQ ID NO: 55 - protein, 12 aa, Artificial
  Antimicrobial peptide
ILRWPWWPWRRK

SEQ ID NO: 56 - protein, 7 aa, Artificial
  Antimicrobial peptide
RRRRWWW

SEQ ID NO: 57 - protein, 7 aa, Artificial
  Antimicrobial peptide
RRWWRRW

SEQ ID NO: 58 - protein, 7 aa, Artificial
  Antimicrobial peptide
RRRWWWR

SEQ ID NO: 59 - protein, 7 aa, Artificial
  Antimicrobial peptide
RWRWRWR

SEQ ID NO: 60 - protein, 24 aa, Artificial
  Antimicrobial peptide
GRLVLEITADEVKALGEALANAKI

SEQ ID NO: 61 - protein, 24 aa, Artificial
  Antimicrobial peptide
GRLVLEITADEVKALGEALANAKI

SEQ ID NO: 62 - protein, 7 aa, Artificial
  Antimicrobial peptide
RRRFWWR

SEQ ID NO: 63 - protein, 7 aa, Artificial
  Antimicrobial peptide
RRWWRRF

SEQ ID NO: 64 - protein, 7 aa, Artificial
  Antimicrobial peptide
RRRWWWF

SEQ ID NO: 65 - protein, 7 aa, Artificial
  Antimicrobial peptide
RWRWRWF

SEQ ID NO: 66 - protein, 7 aa, Artificial
  Antimicrobial peptide
RRRRWWK

SEQ ID NO: 67 - protein, 7 aa, Artificial
  Antimicrobial peptide
RRWWRRK

SEQ ID NO: 68 - protein, 7 aa, Artificial
  Antimicrobial peptide
RRRWWWK

SEQ ID NO: 69 - protein, 7 aa, Artificial
  Antimicrobial peptide
RWRWRWK

SEQ ID NO: 70 - protein, 7 aa, Artificial
  Antimicrobial peptide
RRRKWWK

SEQ ID NO: 71 - protein, 7 aa, Artificial
  Antimicrobial peptide
RRWKRRK

SEQ ID NO: 72 - protein, 7 aa, Artificial
  Antimicrobial peptide
RRRKWWK

SEQ ID NO: 73 - protein, 7 aa, Artificial
  Antimicrobial peptide
RWRKRWK

SEQ ID NO: 74 - protein, 14 aa, Artificial
  Antimicrobial peptide
KLKKLLKRWRRWWR

SEQ ID NO: 75 - protein, 14 aa, Artificial
  Antimicrobial peptide
RWRRLLKKLHHLLH

SEQ ID NO: 76 - protein, 14 aa, Artificial
  Antimicrobial peptide
KLKKLLKHLHHLLH

SEQ ID NO: 77 - protein, 14 aa, Artificial
  Antimicrobial peptide
LHLLHQLLHLLHQF

SEQ ID NO: 78 - protein, 14 aa, Artificial
  Antimicrobial peptide
AQAAHQAAHAAHQF

SEQ ID NO: 79 - protein, 14 aa, Artificial
  Antimicrobial peptide
KLKKLLKKLKKLLK

SEQ ID NO: 80 - protein, 14 aa, Artificial
  Antimicrobial peptide
LKLLKKLLKLLKKF

SEQ ID NO: 81 - protein, 14 aa, Artificial
  Antimicrobial peptide
LQLLKQLLKLLKQF

SEQ ID NO: 82 - protein, 14 aa, Artificial
  Antimicrobial peptide
AQAAKQAAKAAKQF

SEQ ID NO: 83 - protein, 14 aa, Artificial
  Antimicrobial peptide
RWRRWWRHFHHFFH

SEQ ID NO: 84 - protein, 14 aa, Artificial
  Antimicrobial peptide
HHFFHHFHHFFHHF

SEQ ID NO: 85 - protein, 14 aa, Artificial
  Antimicrobial peptide
FHFFHHFFHFFHHF

SEQ ID NO: 86 - protein, 21 aa, Artificial
  Antimicrobial peptide
KLLKGATFHFFHHFFHFFHHF

SEQ ID NO: 87 - protein, 18 aa, Artificial
  Antimicrobial peptide
KLLKFHFFHHFFHFFHHF

SEQ ID NO: 88 - protein, 18 aa, Artificial
  Antimicrobial peptide
FHFFHHFFHFFHHFKLLK

SEQ ID NO: 89 - protein, 13 aa, Artificial
  Antimicrobial peptide
FLKFLKKFFKKLK

SEQ ID NO: 90 - protein, 11 aa, Artificial
  Antimicrobial peptide
FKKFWKWFRRF

SEQ ID NO: 91 - protein, 11 aa, Artificial
  Antimicrobial peptide
LKRFLKWFKRF

SEQ ID NO: 92 - protein, 11 aa, Artificial
  Antimicrobial peptide
KLFKRWKHLFR

SEQ ID NO: 93 - protein, 11 aa, Artificial
  Antimicrobial peptide
RLLKRFKHLFK

SEQ ID NO: 94 - protein, 11 aa, Artificial
  Antimicrobial peptide
FKTFLKWLHRF

SEQ ID NO: 95 - protein, 11 aa, Artificial
  Antimicrobial peptide
IKQLLHFFQRF

SEQ ID NO: 96 - protein, 11 aa, Artificial
  Antimicrobial peptide
KLLQTFKQIFR

SEQ ID NO: 97 - protein, 11 aa, Artificial
  Antimicrobial peptide
RILKELKNLFK

SEQ ID NO: 98 - protein, 11 aa, Artificial
  Antimicrobial peptide
LKQFVHFIHRF

SEQ ID NO: 99 - protein, 11 aa, Artificial
  Antimicrobial peptide
VKTLLHIFQRF

SEQ ID NO: 100 - protein, 11 aa, Artificial
  Antimicrobial peptide
KLVEQLKEIFR

SEQ ID NO: 101 - protein, 11 aa, Artificial
  Antimicrobial peptide
RVLQEIKQILK

SEQ ID NO: 102 - protein, 11 aa, Artificial
  Antimicrobial peptide
VKNLAELVHRF

SEQ ID NO: 103 - protein, 11 aa, Artificial
  Antimicrobial peptide
ATHLLHALQRF

SEQ ID NO: 104 - protein, 11 aa, Artificial
  Antimicrobial peptide
KLAENVKEILR

SEQ ID NO: 105 - protein, 11 aa, Artificial
  Antimicrobial peptide
RALHEAKEALK

SEQ ID NO: 106 - protein, 11 aa, Artificial
  Antimicrobial peptide
FHYFWHWFHRF

SEQ ID NO: 107 - protein, 11 aa, Artificial
  Antimicrobial peptide
LYHFLHWFQRF

SEQ ID NO: 108 - protein, 11 aa, Artificial
  Antimicrobial peptide
YLFQTWQHLFR

SEQ ID NO: 109 - protein, 11 aa, Artificial
  Antimicrobial peptide
YLLTEFQHLFK

SEQ ID NO: 110 - protein, 11 aa, Artificial
  Antimicrobial peptide
FKTFLQWLHRF

SEQ ID NO: 111 - protein, 11 aa, Artificial
  Antimicrobial peptide
IKTLLHFFQRF

SEQ ID NO: 112 - protein, 11 aa, Artificial
  Antimicrobial peptide
KLLQTFNQIFR

SEQ ID NO: 113 - protein, 11 aa, Artificial
  Antimicrobial peptide
TILQSLKNIFK

SEQ ID NO: 114 - protein, 11 aa, Artificial
  Antimicrobial peptide
LKQFVKFIHRF

SEQ ID NO: 115 - protein, 11 aa, Artificial
  Antimicrobial peptide
VKQLLKIFNRF

SEQ ID NO: 116 - protein, 11 aa, Artificial
  Antimicrobial peptide
KLVQQLKNIFR

SEQ ID NO: 117 - protein, 11 aa, Artificial
  Antimicrobial peptide
RVLNQVKQILK

SEQ ID NO: 118 - protein, 11 aa, Artificial
  Antimicrobial peptide
VKKLAKLVRRF

SEQ ID NO: 119 - protein, 11 aa, Artificial
  Antimicrobial peptide
AKRLLKVLKRF

SEQ ID NO: 120 - protein, 11 aa, Artificial
  Antimicrobial peptide
KLAQKVKRVLR

SEQ ID NO: 121 - protein, 11 aa, Artificial
  Antimicrobial peptide
RALKRIKHVLK

SEQ ID NO: 122 - protein, 15 aa, Artificial
  Antimicrobial peptide
FVFRHKWVWKHRFLF

SEQ ID NO: 123 - protein, 15 aa, Artificial
  Antimicrobial peptide
FKAHIRFKLRVKFHF

SEQ ID NO: 124 - protein, 15 aa, Artificial
  Antimicrobial peptide
LKAKIKFKVKLKIKF

SEQ ID NO: 125 - protein, 14 aa, Artificial
  Antimicrobial peptide
WIWKHKFLHRHFLF

SEQ ID NO: 126 - protein, 14 aa, Artificial
  Antimicrobial peptide
VFLHRHVIKHKLVF

SEQ ID NO: 127 - protein, 13 aa, Artificial
  Antimicrobial peptide
FLHKHVLRHRIVF

SEQ ID NO: 128 - protein, 13 aa, Artificial
  Antimicrobial peptide
VFKHKIVHRHILF

SEQ ID NO: 129 - protein, 13 aa, Artificial
  Antimicrobial peptide
FLFKHLFLHRIFF

SEQ ID NO: 130 - protein, 12 aa, Artificial
  Antimicrobial peptide
LFKHILIHRVIF

SEQ ID NO: 131 - protein, 12 aa, Artificial
  Antimicrobial peptide
FLHKHLFKHKLF

SEQ ID NO: 132 - protein, 12 aa, Artificial
  Antimicrobial peptide
VFRHRFIHRHVF

SEQ ID NO: 133 - protein, 15 aa, Artificial
  Antimicrobial peptide
VFIHRHVWVHKHVLF

SEQ ID NO: 134 - protein, 11 aa, Artificial
  Antimicrobial peptide
KLFKFLRKHLL

SEQ ID NO: 135 - protein, 12 aa, Artificial
  Antimicrobial peptide
FIHKLVHKHVLF

SEQ ID NO: 136 - protein, 12 aa, Artificial
  Antimicrobial peptide
VLRHLFRHRIVF

SEQ ID NO: 137 - protein, 12 aa, Artificial
  Antimicrobial peptide
LVHKLILRHLLF

SEQ ID NO: 138 - protein, 12 aa, Artificial
  Antimicrobial peptide
VFKRVLIHKLIF

SEQ ID NO: 139 - protein, 12 aa, Artificial
  Antimicrobial peptide
IVRKFLFRHKVF

SEQ ID NO: 140 - protein, 12 aa, Artificial
  Antimicrobial peptide
VLKHVIAHKRLF

SEQ ID NO: 141 - protein, 11 aa, Artificial
  Antimicrobial peptide
FIRKFLFKHLF

SEQ ID NO: 142 - protein, 11 aa, Artificial
  Antimicrobial peptide
VIRHVWVRKLF

SEQ ID NO: 143 - protein, 13 aa, Artificial
  Antimicrobial peptide
FLFRHRFRHRLVF

SEQ ID NO: 144 - protein, 13 aa, Artificial
  Antimicrobial peptide
LFLHKHAKHKFLF

SEQ ID NO: 145 - protein, 15 aa, Artificial
  Antimicrobial peptide
WRWRARWRWRLRWRF

SEQ ID NO: 146 - protein, 11 aa, Artificial
  Antimicrobial peptide
FKHKFKHKFIF

SEQ ID NO: 147 - protein, 11 aa, Artificial
  Antimicrobial peptide
LRHRLRHRLIF

SEQ ID NO: 148 - protein, 11 aa, Artificial
  Antimicrobial peptide
LILKFLFKFVF

SEQ ID NO: 149 - protein, 11 aa, Artificial
  Antimicrobial peptide
VLIRILVRVIF

SEQ ID NO: 150 - protein, 9 aa, Artificial
  Antimicrobial peptide
FRHRFRHRF

SEQ ID NO: 151 - protein, 9 aa, Artificial
  Antimicrobial peptide
LKHKLKHKF

SEQ ID NO: 152 - protein, 9 aa, Artificial
  Antimicrobial peptide
FKFKHKLIF

SEQ ID NO: 153 - protein, 9 aa, Artificial
  Antimicrobial peptide
LRLRHRVLF

SEQ ID NO: 154 - protein, 9 aa, Artificial
  Antimicrobial peptide
FKFLFKFLF

SEQ ID NO: 155 - protein, 9 aa, Artificial
  Antimicrobial peptide
LRLFLRWLF

SEQ ID NO: 156 - protein, 15 aa, Artificial
  Antimicrobial peptide
WRIHLRARLHVKFRF

SEQ ID NO: 157 - protein, 9 aa, Artificial
  Antimicrobial peptide
FKFLFKHKF

SEQ ID NO: 158 - protein, 9 aa, Artificial
  Antimicrobial peptide
LRLFLRHRF

SEQ ID NO: 159 - protein, 7 aa, Artificial
  Antimicrobial peptide
FKFLFKF

SEQ ID NO: 160 - protein, 7 aa, Artificial
  Antimicrobial peptide
LRLFLRF

SEQ ID NO: 161 - protein, 15 aa, Artificial
  Antimicrobial peptide
LRIHARFKVHIRLKF

SEQ ID NO: 162 - protein, 15 aa, Artificial
  Antimicrobial peptide
FHIKFRVHLKVRFHF

SEQ ID NO: 163 - protein, 15 aa, Artificial
  Antimicrobial peptide
FHVKIHFRLHVKFHF

SEQ ID NO: 164 - protein, 15 aa, Artificial
  Antimicrobial peptide
LHIHAHFHVHIHLHF

SEQ ID NO: 165 - protein, 15 aa, Artificial
  Antimicrobial peptide
FKIHFRLKVHIRFKF

SEQ ID NO: 166 - protein, 15 aa, Artificial
  Antimicrobial peptide
GWRLIKKILRVFKGL

SEQ ID NO: 167 - protein, 38 aa, Artificial
  Antimicrobial peptide
CLLGDFFRKSKEKIGKEFKRIVQRIKDFLRNLVPRTES

SEQ ID NO: 168 - protein, 39 aa, Artificial
  Antimicrobial peptide
CLLGDFFRKSKEKIGKEFKRIVQRIKDFLRNLVPRTESC

SEQ ID NO: 169 - protein, 16 aa, Artificial
  Antimicrobial peptide
KNLRIIRKGIHIIKKY

SEQ ID NO: 170 - protein, 17 aa, Artificial
  Antimicrobial peptide
GLGRVIGRLIKQIIWRR

SEQ ID NO: 171 - protein, 11 aa, Artificial
  Antimicrobial peptide
KILKFLFKQVF

SEQ ID NO: 172 - protein, 11 aa, Artificial
  Antimicrobial peptide
KILKKLFKFVF

SEQ ID NO: 173 - protein, 11 aa, Artificial
  Antimicrobial peptide
GILKKLFTKVF

SEQ ID NO: 174 - protein, 9 aa, Artificial
  Antimicrobial peptide
LRKFLHKLF

SEQ ID NO: 175 - protein, 9 aa, Artificial
  Antimicrobial peptide
LRKNLRWLF

SEQ ID NO: 176 - protein, 11 aa, Artificial
  Antimicrobial peptide
FIRKFLQKLHL

SEQ ID NO: 177 - protein, 11 aa, Artificial
  Antimicrobial peptide
FTRKFLKFLHL

SEQ ID NO: 178 - protein, 12 aa, Artificial
  Antimicrobial peptide
KKFKKFKVLKIL

SEQ ID NO: 179 - protein, 12 aa, Artificial
  Antimicrobial peptide
LLKLLKLKKLKF

SEQ ID NO: 180 - protein, 19 aa, Artificial
  Antimicrobial peptide
VYRKRKSILKIYAKLKGWH

SEQ ID NO: 181 - protein, 14 aa, Artificial
  Antimicrobial peptide
FLKFLKKFFKKLKY

SEQ ID NO: 182 - protein, 16 aa, Artificial
  Antimicrobial peptide
GWLKMFKKIIGKFGKF

SEQ ID NO: 183 - protein, 16 aa, Artificial
  Antimicrobial peptide
GIFKKFVKILYKVQKL

SEQ ID NO: 184 - protein, 20 aa, Artificial
  Antimicrobial peptide
NYRLVNAIFSKIFKKKFIKF

SEQ ID NO: 185 - protein, 11 aa, Artificial
  Antimicrobial peptide
KILKFLFKKVF

SEQ ID NO: 186 - protein, 11 aa, Artificial
  Antimicrobial peptide
FIRKFLKKWLL

SEQ ID NO: 187 - protein, 37 aa, Artificial; identical to SEQ ID NO: 47
  Antimicrobial peptide

SEQ ID NO: 188 - protein, 16 aa, Artificial
  Antimicrobial peptide
FKRIVQRIKDFLRNLV

SEQ ID NO: 189 - protein, 38 aa, Artificial
  Antimicrobial peptide
LLGDFFRKSKEKIGKEFKRIVQRIKDFLRNLVPRTESC

SEQ ID NO: 190 - protein, 13 aa, Artificial
  Antimicrobial peptide
FKRIVQRIKDFLR

SEQ ID NO: 191 - protein, 21 aa, Artificial
  Antimicrobial peptide
FKRIVQRIKDFLRNLVPRTES

SEQ ID NO: 192 - protein, 21 aa, Artificial
  Antimicrobial peptide
GKEFKRIVQRIKDFLRNLVPR

SEQ ID NO: 193 - protein, 20 aa, Artificial
  Antimicrobial peptide
KRIVQRIKDFLRNLVPRTES

SEQ ID NO: 194 - protein, 21 aa, Artificial
  Antimicrobial peptide
LLGDFFRKSKEKIGKEFKRIV

SEQ ID NO: 195 - protein, 31 aa, Artificial
  Antimicrobial peptide
RKSKEKIGKEFKRIVQRIKDFLRNLVPRTES

SEQ ID NO: 196 - protein, 21 aa, Artificial
  Antimicrobial peptide
SKEKIGKEFKRIVQRIKDFLR

SEQ ID NO: 197 - protein, 18 aa, Artificial
  Antimicrobial peptide
KNLRRIIRKGIHIIKKYG

SEQ ID NO: 198 - protein, 18 aa, Artificial
  Antimicrobial peptide
KNLRRIGRKIIHIIKKYG

SEQ ID NO: 199 - protein, 18 aa, Artificial
  Antimicrobial peptide
KNLRRIIRKTIHIIKKYG

SEQ ID NO: 200 - protein, 18 aa, Artificial
  Antimicrobial peptide
KNLRRITRKIIHIIKKYG

SEQ ID NO: 201 - protein, 18 aa, Artificial
  Antimicrobial peptide
KNLRRIIRKIIHIIKKYG

SEQ ID NO: 202 - protein, 21 aa, Artificial
  Antimicrobial peptide
MGIIAGIIKFIKGLIEKFTGK

SEQ ID NO: 203 - protein, 25 aa, Artificial
  Antimicrobial peptide
RRGCTERLRRMARRNAWDLYAEHFY

SEQ ID NO: 204 - protein, 28 aa, Artificial
  Antimicrobial peptide
VLPFPAIPLSRRRACVAAPRPRSRQRAS

SEQ ID NO: 205 - protein, 8 aa, Artificial
  Antimicrobial peptide
NYAVVSHT

SEQ ID NO: 206 - protein, 24 aa, Artificial
  Antimicrobial peptide
LSLATFAKIFMTRSNWSLKRFNRL

SEQ ID NO: 207 - protein, 30 aa, Artificial
  Antimicrobial peptide
MIRIRSPTKKKLNRNSISDWKSNTSGRFFY

SEQ ID NO: 208 - protein, 37 aa, Artificial
  Antimicrobial peptide
MKRRRCNWCGKLFYLEEKSKEAYCCKECRKKAKKVKK

SEQ ID NO: 209 - protein, 18 aa, Artificial
  Antimicrobial peptide
GIVLIGLKLIPLLANVLR

SEQ ID NO: 210 - protein, 23 aa, Artificial
  Antimicrobial peptide
FQKPFTGEEVEDFQDDDEIPTII

SEQ ID NO: 211 - protein, 15 aa, Artificial
  Antimicrobial peptide
SLQSQLGPCLHDQRH

SEQ ID NO: 212 - protein, 22 aa, Artificial
  Antimicrobial peptide
LVLRICTDLFTFIKWTIKQRKS

SEQ ID NO: 213 - protein, 24 aa, Artificial
  Antimicrobial peptide
VYSFLYVLVIVRKLLSMKKRIERL

SEQ ID NO: 214 - protein, 21 aa, Artificial
  Antimicrobial peptide
VMQSLYVKPPLILVTKLAQQN

SEQ ID NO: 215 - protein, 16 aa, Artificial
  Antimicrobial peptide
SFMPEIQKNTIPTQMK

SEQ ID NO: 216 - protein, 39 aa, Artificial
  Antimicrobial peptide
LGLTAGVAYAAQPTNQPTNQPTNQPTNQPTNQPTNQPRW

SEQ ID NO: 217 - protein, 15 aa, Artificial
  Antimicrobial peptide
CGKLLEQKNFFLKTR

SEQ ID NO: 218 - protein, 20 aa, Artificial
  Antimicrobial peptide
FELVDWLETNLGKILKSKSA

SEQ ID NO: 219 - protein, 30 aa, Artificial
  Antimicrobial peptide
ASKQASKQASKQASKQASKQASRSLKNHLL

SEQ ID NO: 220 - protein, 24 aa, Artificial
  Antimicrobial peptide
PDAPRTCYHKPILAALSRIVVTDR

SEQ ID NO: 221 - protein, 20 aa, Artificial
  Antimicrobial peptide
KFSDQIDKGQDALKDKLGDL

SEQ ID NO: 222 - protein, 32 aa, Artificial
  Antimicrobial peptide
GSVIKKRRKRMAKKKHRKLLKKTRIQRRRAGK

SEQ ID NO: 223 - protein, 13 aa, Artificial
  Antimicrobial peptide
LSEMERRRLRKRA

SEQ ID NO: 224 - protein, 26 aa, Artificial
  Antimicrobial peptide
SKFKVLRKIIIKEYKGELMLSIQKQR

SEQ ID NO: 225 - protein, 21 aa, Artificial
  Antimicrobial peptide
YIQFHLNQQPRPKVKKIKIFL

SEQ ID NO: 226 - protein, 30 aa, Artificial
  Antimicrobial peptide
KNKKQTDILEKVKEILDKKKKTKSVGQKLY

SEQ ID NO: 227 - protein, 16 aa, Artificial
  Antimicrobial peptide
RESKLIAMADMIRRRI

SEQ ID NO: 228 - protein, 18 aa, Artificial
  Antimicrobial peptide
WSRVPGHSDTGWKVWHRW

SEQ ID NO: 229 - protein, 28 aa, Artificial
  Antimicrobial peptide
KFQGEFTNIGQSYIVSASHMSTSLNTGK

SEQ ID NO: 230 - protein, 41 aa, Artificial
  Antimicrobial peptide
TKKIELKRFVDAFVKKSYENYILERELKKLIKAINEELPTK

SEQ ID NO: 231 - protein, 39 aa, Artificial
  Antimicrobial peptide
FESKILNASKELDKEKKVNTALSFNSHQDFAKAYQNGKI

SEQ ID NO: 232 - protein, 30 aa, Artificial
  Antimicrobial peptide
MRFGSLALVAYDSAIKHSWPRPSSVRRLRM

SEQ ID NO: 233 - protein, 18 aa, Artificial
  Antimicrobial peptide
GLLRRLRKKIGEIFKKYG

SEQ ID NO: 234 - protein, 18 aa, Artificial
  Antimicrobial peptide
RGGRLCYCRRRFCVCVGR

SEQ ID NO: 235 - protein, 7 aa, Artificial
  Antimicrobial peptide
YSPWTNF

SEQ ID NO: 236 - protein, 18 aa, ARTIFICIAL
  Antimicrobial peptide
KNLRRXXRKXXHIIKKYG

SEQ ID NO: 237 - protein, 16 aa, Artificial
  Antimicrobial peptide
KNLRRGIRKIIHIIKK

SEQ ID NO: 238 - protein, 16 aa, Artificial
  Antimicrobial peptide
KNLRRTIRKIIHIIKK

SEQ ID NO: 239 - protein, 16 aa, Artificial
  Antimicrobial peptide
KNLRRSIRKIIHIIKK

SEQ ID NO: 240 - protein, 16 aa, Artificial
  Antimicrobial peptide
KNLRREIRKIIHIIKK

SEQ ID NO: 241 - protein, 16 aa, Artificial
  Antimicrobial peptide
KNLRRDIRKIIHIIKK

SEQ ID NO: 242 - protein, 16 aa, Artificial
  Antimicrobial peptide
KNLRRAIRKIIHIIKK

SEQ ID NO: 243 - protein, 16 aa, Artificial
  Antimicrobial peptide
KNLRRIIRKIIHIIKK

SEQ ID NO: 244 - protein, 16 aa, Artificial
  Antimicrobial peptide
KNLRRIGRKIIHIIKK

SEQ ID NO: 245 - protein, 16 aa, Artificial
  Antimicrobial peptide
KNLRRITRKIIHIIKK

SEQ ID NO: 246 - protein, 16 aa, Artificial
  Antimicrobial peptide
KNLRRISRKIIHIIKK

SEQ ID NO: 247 - protein, 16 aa, Artificial
  Antimicrobial peptide
KNLRRIERKIIHIIKK

SEQ ID NO: 248 - protein, 16 aa, Artificial
  Antimicrobial peptide
KNLRRIDRKIIHIIKK

SEQ ID NO: 249 - protein, 16 aa, Artificial
  Antimicrobial peptide
KNLRRIARKIIHIIKK

SEQ ID NO: 250 - protein, 16 aa, Artificial
  Antimicrobial peptide
KNLRRIIRKIIHIIKK

SEQ ID NO: 251 - protein, 16 aa, Artificial
  Antimicrobial peptide
KNLRRIIRKGIHIIKK

SEQ ID NO: 252 - protein, 16 aa, Artificial
  Antimicrobial peptide
KNLRRIIRKTIHIIKK

SEQ ID NO: 253 - protein, 16 aa, Artificial
  Antimicrobial peptide
KNLRRIIRKSIHIIKK

SEQ ID NO: 254 - protein, 16 aa, Artificial
  Antimicrobial peptide
KNLRRIIRKEIHIIKK

SEQ ID NO: 255 - protein, 16 aa, Artificial
  Antimicrobial peptide
KNLRRIIRKDIHIIKK

SEQ ID NO: 256 - protein, 16 aa, Artificial
  Antimicrobial peptide
KNLRRIIRKAIHIIKK

SEQ ID NO: 257 - protein, 16 aa, Artificial
  Antimicrobial peptide
KNLRRIIRKIIHIIKK

SEQ ID NO: 258 - protein, 16 aa, Artificial
  Antimicrobial peptide
KNLRRIIRKIGHIIKK

SEQ ID NO: 259 - protein, 16 aa, Artificial
  Antimicrobial peptide
KNLRRIIRKITHIIKK

SEQ ID NO: 260 - protein, 16 aa, Artificial
  Antimicrobial peptide
KNLRRIIRKISHIIKK

SEQ ID NO: 261 - protein, 16 aa, Artificial
  Antimicrobial peptide
KNLRRIIRKIEHIIKK

SEQ ID NO: 262 - protein, 8 aa, Artificial
  Antimicrobial peptide
DYKDDDDK

SEQ ID NO: 263 - protein, 4 aa, Artificial
  Antimicrobial peptide
HHHH

SEQ ID NO: 264 - protein, 5 aa, Artificial
  Antimicrobial peptide
HHHHH

SEQ ID NO: 265 - protein, 6 aa, Artificial
  Antimicrobial peptide
HHHHHH

SEQ ID NO: 266 - protein, 4 aa, Artificial
  Peptide linker
GGGG

SEQ ID NO: 267 - protein, 5 aa, Artificial
  Peptide linker
GGGGG

SEQ ID NO: 268 - protein, 6 aa, Artificial
  Peptide linker
GGGGGG

SEQ ID NO: 269 - protein, 7 aa, Artificial
  Peptide linker
GGGGGGG

SEQ ID NO: 270 - protein, 8 aa, Artificial
  Peptide linker
GGGGGGGG

SEQ ID NO: 271 - protein, 4 aa, Artificial
  Peptide linker
GGAG

SEQ ID NO: 272 - protein, 5 aa, Artificial
  Peptide linker
GGGAG

SEQ ID NO: 273 - protein, 4 aa, Artificial
  Peptide linker
GSGS

SEQ ID NO: 274 - protein, 6 aa, Artificial
  Peptide linker
GSGSGS

SEQ ID NO: 275 - protein, 8 aa, Artificial
  Peptide linker
GSGSGSGS

SEQ ID NO: 276 - protein, 10 aa, Artificial
  Peptide linker
GSGSGSGSGS

SEQ ID NO: 277 - protein, 12 aa, Artificial
  Peptide linker
GSGSGSGSGSGS

SEQ ID NO: 278 - protein, 5 aa, Artificial
  Peptide linker
PSPSP

SEQ ID NO: 279 - protein, 4 aa, Artificial
  Peptide linker
KKKK

SEQ ID NO: 280 - protein, 4 aa, Artificial
  Peptide linker
RRRR

SEQ ID NO: 281 - protein, 5 aa, Artificial
  Peptide linker
ASASA

SEQ ID NO: 282 - protein, 5 aa, Artificial
  Peptide linker
PSGSP

SEQ ID NO: 283 - protein, 6 aa, Artificial
  Peptide linker
GGSGGS

SEQ ID NO: 284 - protein, 5 aa, Artificial
  Peptide linker
GGGGS

SEQ ID NO: 285 - protein, 10 aa, Artificial
  Peptide linker
GGGGSGGGGS

SEQ ID NO: 286 - protein, 15 aa, Artificial
  Peptide linker
GGGGSGGGGSGGGGS

SEQ ID NO: 287 - protein, 20 aa, Artificial
  Peptide linker
GGGGSGGGGSGGGGSGGGGS

SEQ ID NO: 288 - protein, 25 aa, Artificial
  Peptide linker
GGGGSGGGGSGGGGSGGGGSGGGGS

SEQ ID NO: 289 - protein, 30 aa, Artificial
  Peptide linker
GGGGSGGGGSGGGGSGGGGSGGGGSGGGGS

SEQ ID NO: 290 - protein, 6 aa, Artificial
  Chemoattractant peptide
XKYXXM

SEQ ID NO: 291 - protein, 6 aa, Artificial
  Chemoattractant peptide
WLYMVM

SEQ ID NO: 292 - protein, 6 aa, Artificial
  Chemoattractant peptide
WKYMVX

SEQ ID NO: 293 - protein, 29 aa, Artificial
  Antimicrobial peptide
VPAKLLRVIDEIPGGFLKFLKKFFKKLKY

SEQ ID NO: 294 - protein, 28 aa, Artificial
  Antimicrobial peptide
VPAKLLRVIKKIPGGFLKFLKKFFKKLK

SEQ ID NO: 295 - protein, 28 aa, Artificial
  Antimicrobial peptide
VPAKLLRVIKEIPGGFLKFLKKFFKKLK

SEQ ID NO: 296 - protein, 24 aa, Artificial
  Antimicrobial peptide
VPAKLLRVIDEIPKLFKFLRKHLL

SEQ ID NO: 297 - protein, 34 aa, Artificial
  Antimicrobial peptide
VPAKLLRVIDEIPGGKIFGAIWPLALGALKNLIK

SEQ ID NO: 298 - protein, 33 aa, Artificial
  Antimicrobial peptide
LATKLKYEKEHKKMGGGGFLKFLKKFFKKLKYY

SEQ ID NO: 299 - protein, 34 aa, Artificial
  Antimicrobial peptide
LATKLKYEKEHKKMGKIFGAIWPLALGALKNLIK

SEQ ID NO: 300 - protein, 34 aa, Artificial
  Antimicrobial peptide
LATLKKYLKEHKKMGKIFGAIWPLALGALKNLIK

SEQ ID NO: 301 - protein, 30 aa, Artificial
  Antimicrobial peptide
NILRVLKQVWKGGGKNLRIIRKGIHIIKKY

SEQ ID NO: 302 - protein, 31 aa, Artificial
  Antimicrobial peptide
GNFYRLFKDILKGGGKNLRIIRKGIHIIKKY

SEQ ID NO: 303 - protein, 21 aa, Artificial
  Antimicrobial peptide
PGGGLLRRLRKKIGEIFKKYG

(19) United States
(12) Patent Application Publication
(10) Pub. No.: US 2016/0207969 A1
(43) Pub. Date: Jul. 21, 2016

(54) CLOSTRIDIUM DIFFICILE TARGETING MOIETIES AND CONSTRUCTS COMPRISING SAID MOIETIES

(71) Applicant: C3 Jian, Inc., Marina Del Rey, CA (US)

(72) Inventors: Shaoying Lee, Los Angeles, CA (US); Miroslav Baudys, Marina Del Rey, CA (US); Randal H. Eckert, Rancho Palos Verdes, CA (US); Brian C. Varnum, Santa Monica, CA (US); Vlad Omel, Los Angeles, CA (US)

(21) Appl. No.: 14/981,462

(22) Filed: Dec. 28, 2015

Related U.S. Application Data
(60) Provisional application No. 62/097,529, filed on Dec. 29, 2014.

(51) Int. Cl. C07K 14/435

(52) U.S. Cl. CPC C07K 14/435; A61K 38/00

(57) ABSTRACT

In certain embodiments, constructs are provided that selectively/preferentially inhibit and/or kill Clostridium difficile. In certain embodiments the constructs comprise a peptide that binds C. difficile attached directly or through an amino acid or a linker to an antimicrobial peptide.

### CROSS-REFERENCE TO RELATED APPLICATIONS

This application claims benefit of and priority to U.S. Ser. No. 62/097,529, filed on Dec. 29, 2014, which is incorporated herein by reference in its entirety for all purposes.

### STATEMENT OF GOVERNMENTAL SUPPORT

[Not Applicable]

### BACKGROUND

Clostridium difficile is an anaerobic, spore-forming bacterium that is able to colonize the human gut, with the resultant infection causing a wide range of symptoms including, but not limited to mild to severe diarrhea, blood-stained stools, abdominal cramps, and fever that in severe cases can be fatal (Johnson (2009) J. Infection, 58: 403-410). It is the major cause of antibiotic-associated pseudo-membranous colitis and diarrhea in human.

Clostridium difficile is an emerging pathogen of opportunistic infection in hospitals worldwide. The incidence and severity of infection are strongly correlated with the extent and duration of antibiotic therapy that patients receive. This is because most antibiotics used to treat the gut are broad-spectrum and kill both pathogenic and non-pathogenic bacteria alike. The effect of this sterilization is that, with reduced competition by other bacterial species, dormant spores of the commensal C. difficile are able to germinate and rapidly colonize the gut.

As the C. difficile infection proceeds, the bacteria begin to produce toxins (Toxin A (tcdA) and B (TcdB)) which disrupt intestinal epithelial cells. Toxin A and Toxin B are believed to enter the cells through receptor-mediated endocytosis and disrupt normal signaling pathways necessary for maintaining the cells' cytoskeleton, ultimately leading to inflammation and diarrhea. These toxins cause a strong inflammatory response that may lead to Pseudomembranous colitis (PMC) and/or may give rise to the symptoms associated with the infection (Bartlett et al. (1977) J. Infect. Dis. 136: 701-705; McDonald et al. (2005) N. Eng. J. Med. 353: 2433-2441).

In the later stages of the infection, the bacteria once again produce endospores that are shed, along with live bacteria, in the feces and greatly increase the chance of re-infection of the patient or spread to fellow patients. Hence, the recurrence rate of the disease is estimated to be between 20 and 45% following resolution of initial treatment (Aslam et al. (2005) Lancet Infect. Dis. 5: 549-557; McFarland et al. (2002) Am. J. Gasteroenterol. 97: 1769-1775).

C. difficile occurs naturally in the gut microflora of newborns, a minority of the adult population below 65, and the majority of those over 65. Hence, C. difficile-associated disease (CDAD) is also correlated with elderly patients, especially those resident in nursing homes or hospitals, as well as all patients who have undergone gastrointestinal surgery or are immuno-compromised. As such C. difficile is a major cause of nosocomial infections. In the United Kingdom in 2003, twice as many deaths were attributable to CDAD as MRSA (Kuijper et al. (2006) Clin. Microbiol. Infection, 12(Supplement 6): 2-18), and approximately three billion euros was spent on treating CDAD across the EU as a whole. In the US, the annual costs associated with CDAD are estimated as $3.2 billion (O'Brien et al. (2007) Infect. Control Hosp. Epidemiol. 28: 1217-1219-1227). An estimated additional 7,147 Euros is spent per CDAD patient in the European Union, compared to the cost of treating a non-CDAD patient (Vonberg et al. (2008) J. Hosp. Infect. 70: 15-20), which equates to an estimated additional annual spend of 350 million in the UK where 5 of every 1000 patient days are attributable to CDAD (Bauer et al. (2011) Lancet 377: 63-73).

Globally, the incidence and severity of CDAD is increasing (Dubberke et al. (2010) Infect. Control Hosp. Epidemol. 10: 1030-1037). This can be linked to the emergence of new types of hyper-virulent strains, most notably PCR-ribotype 027, that overproduces Toxins A and B and induces severe diarrhea (Warny et al. (2005) Lancet 366: 1079-1084) and results in increased rates of mortality (Pepin et al. (2005) CMAJ, 173: 1037-1042). The PCR-ribotype 027 strain was first recognized as causing an outbreak in the Stoke Mandeville Hospital (UK) where it was responsible for 174 cases of CDAD and 19 (11%) deaths (Anon. (2005) CDR Weekly 15: 24). Within 2 years, the strain had been detected in most EU countries and North America (Freeman et al. (2010) Clin. Microbiol. Rev. 23: 529-549). Currently there are ten commonly recognized PCR ribotypes of C. difficile that have been implicated in hospital outbreaks, all with differing antibiotic sensitivities and virulence (Huang et al. (2009) Int. J. Antimicrob. Agents, 34: 516-522). A particular challenge is ribotypes that show resistance to fluoroquinolines, such as 027 (McDonald et al. (2005) N. Eng. J. Med. 353: 2433-2441). Further, the type of PCR-ribotypes causing infections can differ between hospitals in the same country and change quickly. For example, in the European Union, as the incidence of the hyper-virulent strain wanes (PCR-ribotype 027) another one waxes (PCR-ribotype 078) (O'Donoghue and Kyne (2011) Curr. Opin. Gastroenterol. 27: 38-47). This emphasizes the rapidity with which the epidemiology of CDAD can change and the extent of the challenge to develop robust and effective therapeutics and treatment strategies to respond to the varied threat of C. difficile infection.

### SUMMARY

Novel peptides were identified that preferentially or specifically bind Clostridium difficile. These “targeting peptides” can be attached to various effectors for preferential/specific delivery to C. difficile in vivo or in vitro. In certain embodiments the targeting peptides are attached to antimicrobial peptides to thereby form a specifically targeted antimicrobial peptide (a STAMP). In certain embodiments the targeting peptide can be attached to a chemoattractant peptide (e.g., a leukocyte chemoattractant peptide) as described herein. In certain embodiments the STAMP (targeting peptide attached to antimicrobial peptide) is attached to a chemoattractant peptide described herein.

Various embodiments contemplated herein may include, but need not be limited to, one or more of the following:

### Embodiment 1

A targeting peptide that binds to Clostridium difficile, where said peptide includes or consists of an amino acid sequence selected from the group consisting of:

VPAKLLRVIDEIP (SEQ ID NO:3) where said peptide does not comprise the amino acid sequence VPAKLLRVIDEIPE (SEQ ID NO:2); VPAKLLRVIKKIP (SEQ ID NO:4), VPAKLLRVIKEIP (SEQ ID NO:5), NILRVLKQVWK (SEQ ID NO:20), GNFYRLFKDILK (SEQ ID NO:28);

a fragment of VPAKLLRVIDEIP (SEQ ID NO:3) including at least 6, or at least 8, or at least 10 contiguous residues of said sequence where said peptide does not comprise the amino acid sequence VPAKLLRVIDEIPE (SEQ ID NO:2);

a fragment of VPAKLLRVIKKIP (SEQ ID NO:4) including at least 6, or at least 8, or at least 10 contiguous residues of said sequence; a fragment of NILRVLKQVWK (SEQ ID NO:20) including at including at least 6, or at least 8, or at least 10 contiguous residues of said sequence; a fragment of GNFYRLFKDILK (SEQ ID NO:28) including at least 6, or at least 8, or at least 10 contiguous residues of said sequence;

an inverso form of any of the preceding amino acid sequences; and

an amino acid sequence that has at least 90% sequence identity with an amino acid sequence selected from the group consisting of VPAKLLRVIDEIP (SEQ ID NO:3), VPAKLLRVIKKIP (SEQ ID NO:4), VPAKLLRVIKEIP (SEQ ID NO:5), NILRVLKQVWK (SEQ ID NO:20), and GNFYRLFKDILK (SEQ ID NO:28), and where said sequence does not contain the amino acid sequence VPAKLLRVIDEIPE (SEQ ID NO:2);

wherein said targeting peptide binds to C. difficile.

### Embodiment 2

The targeting peptide of embodiment 1, wherein the amino acid sequence of said targeting peptide comprises the sequence VPAKLLRVIDEIP (SEQ ID NO:3) where said peptide does not comprise the amino acid sequence VPAKLLRVIDEIPE (SEQ ID NO:2).

### Embodiment 3

The targeting peptide of embodiment 1, wherein the amino acid sequence of said targeting peptide comprises the amino acid sequence VPAKLLRVIKKIP (SEQ ID NO:4).

### Embodiment 4

The targeting peptide of embodiment 1, wherein the amino acid sequence of said targeting peptide comprises the amino acid sequence VPAKLLRVIKEIP (SEQ ID NO:5).

### Embodiment 5

The targeting peptide of embodiment 1, wherein the amino acid sequence of said targeting peptide comprises the amino acid sequence NILRVLKQVWK (SEQ ID NO:20).

### Embodiment 6

The targeting peptide of embodiment 1, wherein the amino acid sequence of said targeting peptide comprises the amino acid sequence GNFYRLFKDILK (SEQ ID NO:28).

### Embodiment 7

The targeting peptide of embodiment 1, wherein the amino acid sequence of said targeting peptide comprises a fragment of the amino acid sequence VPAKLLRVIDEIP (SEQ ID NO:3) comprising at least 8 contiguous residues of said sequence.

### Embodiment 8

The targeting peptide of embodiment 1, wherein the amino acid sequence of said targeting peptide comprises a fragment of the amino acid sequence VPAKLLRVIKKIP (SEQ ID NO:4) comprising at least 8 contiguous residues of said sequence.

### Embodiment 9

The targeting peptide of embodiment 1, wherein the amino acid sequence of said targeting peptide comprises a fragment of the amino acid sequence VPAKLLRVIKEIP (SEQ ID NO:5) comprising at least 8 contiguous residues of said sequence.

### Embodiment 10

The targeting peptide of embodiment 1, wherein the amino acid sequence of said targeting peptide comprises a fragment of the amino acid sequence NILRVLKQVWK (SEQ ID NO:20) comprising at least 8 contiguous residues of said sequence.

### Embodiment 11

The targeting peptide of embodiment 1, wherein the amino acid sequence of said targeting peptide comprises a fragment of the amino acid sequence GNFYRLFKDILK (SEQ ID NO:28) comprising at least 8 contiguous residues of said sequence.

### Embodiment 12

The targeting peptide of embodiment 1, wherein the amino acid sequence of said targeting peptide comprises the inverse of the sequence VPAKLLRVIDEIP (SEQ ID NO:3) where said peptide does not comprise the amino acid sequence VPAKLLRVIDEIPE (SEQ ID NO:2).

### Embodiment 13

The targeting peptide of embodiment 1, wherein the amino acid sequence of said targeting peptide comprises the inverse of the amino acid sequence VPAKLLRVIKKIP (SEQ ID NO:4).

### Embodiment 14

The targeting peptide of embodiment 1, wherein the amino acid sequence of said targeting peptide comprises the inverse of the amino acid sequence VPAKLLRVIKEIP (SEQ ID NO:5).

### Embodiment 15

The targeting peptide of embodiment 1, wherein the amino acid sequence of said targeting peptide comprises the inverse of the amino acid sequence NILRVLKQVWK (SEQ ID NO:20).

### Embodiment 16

The targeting peptide of embodiment 1, wherein the amino acid sequence of said targeting peptide comprises the inverse of the amino acid sequence GNFYRLFKDILK (SEQ ID NO:28).

### Embodiment 17

The targeting peptide of embodiment 1, wherein the amino acid sequence of said targeting peptide comprises the inverse of a fragment of the amino acid sequence VPAKLLRVIDEIP (SEQ ID NO:3) comprising the inverso of at least 8 contiguous residues of said sequence.

### Embodiment 18

The targeting peptide of embodiment 1, wherein the amino acid sequence of said targeting peptide comprises the inverse of a fragment of the amino acid sequence VPAKLLRVIKKIP (SEQ ID NO:4) comprising the inverso of at least 8 contiguous residues of said sequence.

### Embodiment 19

The targeting peptide of embodiment 1, wherein the amino acid sequence of said targeting peptide comprises the inverse of a fragment of the amino acid sequence VPAKLLRVIKEIP (SEQ ID NO:5) comprising the inverso of at least 8 contiguous residues of said sequence.

### Embodiment 20

The targeting peptide of embodiment 1, wherein the amino acid sequence of said targeting peptide comprises the inverse of a fragment of the amino acid sequence NILRVLKQVWK (SEQ ID NO:20) comprising the inverso of at least 8 contiguous residues of said sequence.

### Embodiment 21

The targeting peptide of embodiment 1, wherein the amino acid sequence of said targeting peptide comprises the inverse of a fragment of the amino acid sequence GNFYRLFKDILK (SEQ ID NO:28) comprising the inverso of at least 8 contiguous residues of said sequence.

### Embodiment 22

The targeting peptide of embodiment 1, wherein the amino acid sequence of said targeting peptide consists of the amino acid sequence VPAKLLRVIDEIP (SEQ ID NO:3).

### Embodiment 23

The targeting peptide of embodiment 1, wherein the amino acid sequence of said targeting peptide consists of the amino acid sequence VPAKLLRVIKKIP (SEQ ID NO:4).

### Embodiment 24

The targeting peptide of embodiment 1, wherein the amino acid sequence of said targeting peptide consists of the amino acid sequence VPAKLLRVIKEIP (SEQ ID NO:5).

### Embodiment 25

The targeting peptide of embodiment 1, wherein the amino acid sequence of said targeting peptide consists of the amino acid sequence NILRVLKQVWK (SEQ ID NO:20).

### Embodiment 26

The targeting peptide of embodiment 1, wherein the amino acid sequence of said targeting peptide consists of the amino acid sequence GNFYRLFKDILK (SEQ ID NO:28).

### Embodiment 27

The targeting peptide according to any one of embodiments 1-21, wherein said targeting peptide ranges in length up to 50 amino acids, or said targeting peptide ranges in length up to about 25 amino acids, or said targeting peptide ranges in length up to about 15 amino acids.

### Embodiment 28

The targeting peptide according to any one of embodiments 1-27, wherein the N terminal amino acid of said targeting peptide is a “D” amino acid.

### Embodiment 29

The targeting peptide according to any one of embodiments 1-27, wherein the C terminal amino acid of said targeting peptide is a “D” amino acid.

### Embodiment 30

The targeting peptide according to any one of embodiments 1-27, wherein said targeting peptide is an “L” peptide.

### Embodiment 31

The targeting peptide according to any one of embodiments 1-27, wherein said targeting peptide is a “D” peptide.

### Embodiment 32

The targeting peptide according to any one of embodiments 1-27, wherein said targeting peptide is a beta peptide.

### Embodiment 33

The targeting peptide according to any one of embodiments 1-31, where said peptide is recombinantly expressed.

### Embodiment 34

The targeting peptide according to any one of embodiments 1-32, where said peptide is chemically synthesized.

### Embodiment 35

The targeting peptide according to any one of embodiments 1-34, where said peptide is purified ex vivo.

### Embodiment 36

The targeting peptide according to any one of embodiments 1-35, wherein said peptide is attached to an effector moiety selected from the group consisting of a detectable label, a porphyrin or other photosensitizer, an antimicrobial peptide, an antibiotic, a ligand, a lipid or liposome, an agent that physically disrupts the extracellular matrix within a community of microorganisms, a chemoattractant peptide, and a polymeric particle.

### Embodiment 37

The targeting peptide of embodiment 36, wherein said targeting peptide is attached to a chemoattractant peptide comprising or consisting of the motif XKYX(P/V)M (SEQ ID NO:289) where X is any amino acid and M is methionine or D-methionine.

### Embodiment 38

The targeting peptide of embodiment 37, wherein the chemoattractant peptide comprises or consists of the amino acid sequence WKYMVM (SEQ ID NO:290), where sequence consists of all “L” amino acids, or where the sequence consists of all D amino acids.

### Embodiment 39

The targeting peptide of embodiment 37, wherein the chemoattractant peptide comprises or consists of the amino acid sequence the chemoattractant peptide comprises or consists of the amino acid sequence WKYMV(dM) (SEQ ID NO: 291) where dM is D-methionine and the other resides are all L residues.

### Embodiment 40

The peptide of embodiment 36, wherein said peptide is attached to an antimicrobial peptide.

### Embodiment 41

A construct comprising: a targeting peptide according to any one of embodiments 1-35, or a targeting peptide comprising or consisting of the amino acid sequence LATKLKYEKEHKKM (SEQ ID NO:11) or that comprises or consists of the amino acid sequence LATLKKYLKEHKKM (SEQ ID NO:12), where said targeting peptide is attached to an effector moiety selected from the group consisting of a detectable label, a porphyrin or other photosensitizer, an antimicrobial peptide, an antibiotic, a ligand, a lipid or liposome, an agent that physically disrupts the extracellular matrix within a community of microorganisms, and a polymeric particle.

### Embodiment 42

The construct of embodiment 41, wherein said targeting peptide is attached to an antimicrobial peptide comprising or consisting of an amino acid sequence found in Table 3.

### Embodiment 43

The construct of embodiment 41, wherein said targeting peptide is attached to an antimicrobial peptide comprising or consisting of an amino acid sequence selected from the group consisting of KNLRIIRKGIHIIKKY ((SEQ ID NO:169, G2), FLKFLKKFFKKLKYY (SEQ ID NO:42), KNLRRIIRKGIHIIKKYG (SEQ ID NO:197), Novispirin G10), KNLRRIIRKTIHIIKKYG (SEQ ID NO:198, Novispirin T10), KNLRRIGRKIIHIIKKYG (SEQ ID NO:199, Novispirin G7), KNLRRITRKIIHIIKKYG (SEQ ID NO:200, Novispirin T7), KNLRRIIRKIIHIIKKYG (SEQ ID NO:201, Ovispirin), PGGGLLRRLRKKIGEIFKKYG (SEQ ID NO:302), RGGRLCYCRRRFCVCVGR (SEQ ID NO:234, Protegrin-1), GLGRVIGRLIKQIIWRR (SEQ ID NO:170, K-1), VYRKRKSILKIYAKLKGWH (SEQ ID NO:180, K-2), NYRLVNAIFSKIFKKKFIKF (SEQ ID NO:184, K-7), KILKFLFKKVF (SEQ ID NO:185, K-8), FIRKFLKKWLL (SEQ ID NO:186, K-9), KLFKFLRKHLL (SEQ ID NO:134, K-10), KILKFLFKQVF (SEQ ID NO:171, K-11), KILKKLFKFVF (SEQ ID NO:172, K-12), GILKKLFTKVF (SEQ ID NO:173, K-13), LRKFLHKLF (SEQ ID NO:174, K-14), LRKNLRWLF (SEQ ID NO:175, K-15), FIRKFLQKLHL (SEQ ID NO:176, K-16), FTRKFLKFLHL (SEQ ID NO:177, K-17), KKFKKFKVLKIL (SEQ ID NO:178, K-18), LLKLLKLKKLKF (SEQ ID NO:179, K-19), FLKFLKKFFKKLKY (SEQ ID NO:181, K-20), GWLKMFKKIIGKFGKF (SEQ ID NO:182, K-21), GIFKKFVKILYKVQKL (SEQ ID NO: 183, K-22), FKKFWKWFRRF (SEQ ID NO:90, B-33), FELVDWLETNLGKILKSKSA (SEQ ID NO:218, PF-522), and YIQFHLNQQPRPKVKKIKIFL (SEQ ID NO: 225, PF-531).

### Embodiment 44

The construct of embodiment 41, wherein said targeting peptide is attached to an antimicrobial peptide comprising or consisting of an amino acid sequence selected from the group consisting of KNLRIIRKGIHIIKKY (SEQ ID NO:169, G2), FLKFLKKFFKKLKY (SEQ ID NO:181), FLKFLKKFFKKLK (SEQ ID NO:89), KLFKFLRKHLL (SEQ ID NO:134), KIFGAIWPLALGALKNLIK (SEQ ID NO:41), FLKFLKKFFKKLKYY (SEQ ID NO:42), KNLRRIIRKGIHIIKKYG (SEQ ID NO: WFLKFLKKFFKKLKY (SEQ ID NO:51), and WFLKFLKKFFKKLK (SEQ ID NO:52).

### Embodiment 45

The construct of embodiment 44, wherein said targeting peptide is attached to an antimicrobial peptide comprising or consisting of the amino acid sequence of G2 KNLRIIRKGIHIIKKY (SEQ ID NO:169).

### Embodiment 46

The construct of embodiment 44, wherein said targeting peptide is attached to an antimicrobial peptide comprising or consisting of the amino acid sequence FLKFLKKFFKKLKY (SEQ ID NO:181).

### Embodiment 47

The construct of embodiment 44, wherein said targeting peptide is attached to an antimicrobial peptide comprising or consisting of the amino acid sequence FLKFLKKFFKKLK (SEQ ID NO:89).

### Embodiment 48

The construct of embodiment 44, wherein said targeting peptide is attached to an antimicrobial peptide comprising or consisting of the amino acid sequence KLFKFLRKHLL (SEQ ID NO:134).

### Embodiment 49

The construct of embodiment 44, wherein said targeting peptide is attached to an antimicrobial peptide comprising or consisting of the amino acid sequence KIFGAIWPLALGALKNLIK (SEQ ID NO:41).

### Embodiment 50

The construct of embodiment 44, wherein said targeting peptide is attached to an antimicrobial peptide comprising or consisting of the amino acid sequence FLKFLKKFFKKLKYY (SEQ ID NO:42).

### Embodiment 51

The construct of embodiment 44, wherein said targeting peptide is attached to an antimicrobial peptide comprising or consisting of the amino acid sequence WFLKFLKKFFKKLKY (SEQ ID NO:51).

### Embodiment 52

The construct of embodiment 44, wherein said targeting peptide is attached to an antimicrobial peptide comprising or consisting of the amino acid sequence WFLKFLKKFFKKLK (SEQ ID NO:52).

### Embodiment 53

The construct of embodiment 44, wherein said targeting peptide is attached to an antimicrobial peptide comprising or consisting of the amino acid sequence Novispirin G10, KNLRRIIRKGIHIIKKYG (197).

### Embodiment 54

The construct according to any one of embodiments 41-53, wherein said targeting peptide and said antimicrobial peptide are “L” peptides.

### Embodiment 55

The construct according to any one of embodiments 41-53, wherein the carboxyl terminal residue of said construct is a “D” amino acid.

### Embodiment 56

The construct according to any one of embodiments 41-53, wherein the carboxyl terminal residue of said construct is a “D: amino acid.

### Embodiment 57

The construct according to any one of embodiments 41-53, wherein said targeting peptide and said antimicrobial peptide are “D” peptides.

### Embodiment 58

The construct according to any one of embodiments 41-53, wherein said targeting peptide and said antimicrobial peptide are beta peptides.

### Embodiment 59

The construct according to any of embodiments 41-58, wherein said targeting peptide is chemically conjugated to said effector.

### Embodiment 60

The construct of embodiment 59, wherein said targeting peptide is chemically conjugated to said effector via a linker.

### Embodiment 61

The construct of embodiment 60, wherein said targeting peptide is chemically conjugated to said effector via a linker comprising a polyethylene glycol (PEG).

### Embodiment 62

The construct of embodiment 60, wherein said targeting peptide is chemically conjugated to said effector via a non-peptide linker found in Table 6.

### Embodiment 63

The construct according to any of embodiments 41-58, wherein said targeting peptide is linked directly to said effector (i.e., without a linker).

### Embodiment 64

The construct according to any of embodiments 41-58, wherein said targeting peptide is linked to said effector via a single amino acid or via a peptide linkage.

### Embodiment 65

The construct of embodiment 64, wherein said effector comprises an antimicrobial peptide and said construct is a fusion protein.

### Embodiment 66

The construct according to any one of embodiments 64 and 65, wherein said targeting peptide is attached to said effector by a single amino acid or by a peptide linker comprising or consisting of an amino acid sequence found in Table 6.

### Embodiment 67

The construct of embodiment 66, wherein said peptide linker comprises or consists of an amino acid or an amino acid sequence selected from the group consisting of G, GG, GGG, and GGGG (SEQ ID NO:265).

### Embodiment 68

The construct of embodiment 41, wherein the amino acid sequence of said construct comprises or consists of an amino acid sequence selected from the group consisting of VPAKLLRVIDEIPGGFLKFLKKFFKKLKY (SEQ ID NO:292, CD0714+AF5_2G), VPAKLLRVIKKIPGGFLKFLKKFFKKLK (SEQ ID NO:293, CD0714+AF5_2G_M4), VPAKLLRVIKEIPGGFLKFLKKFFKKLK (SEQ ID NO:294, CD0714+AF5_2G_M7), VPAKLLRVIDEIPKLFKFLRKHLL (SEQ ID NO:295, CD0714+BD2-21_NG), VPAKLLRVIDEIPGGKIFGAIWPLALGALKNLIK (SEQ ID NO:296, CD0714+Lys_A1_2G), LATKLKYEKEHKKMGGGGFLKFLKKFFKKLKYY (SEQ ID NO:297, CD0126+AF5_4G), LATKLKYEKEHKKMGKIFGAIWPLALGALKNLIK (SEQ ID NO:298, CD0126+Lys_A1_1G), LATLKKYLKEHKKMGKIFGAIWPLALGALKNLIK (SEQ ID NO:299, CD0126+Lys_A1_M3), NILRVLKQVWKGGGKNLRIIRKGIHIIKKY (SEQ ID NO:300, CD9232_G2_3G), and GNFYRLFKDILKGGGKNLRIIRKGIHIIKKY (SEQ ID NO:301, CD8040-G2_3G).

### Embodiment 69

The construct of embodiment 68, wherein the amino acid sequence of said construct consists of the amino acid sequence VPAKLLRVIDEIPGGFLKFLKKFFKKLKY (SEQ ID NO:292).

### Embodiment 70

The construct of embodiment 68, wherein the amino acid sequence of said construct consists of the amino acid sequence VPAKLLRVIKKIPGGFLKFLKKFFKKLK (SEQ ID NO:293).

### Embodiment 71

The construct of embodiment 68, wherein the amino acid sequence of said construct consists of the amino acid sequence VPAKLLRVIKEIPGGFLKFLKKFFKKLK (SEQ ID NO:294).

### Embodiment 72

The construct of embodiment 68, wherein the amino acid sequence of said construct consists of the amino acid sequence VPAKLLRVIDEIPKLFKFLRKHLL (SEQ ID NO:295).

### Embodiment 73

The construct of embodiment 68, wherein the amino acid sequence of said construct consists of the amino acid sequence VPAKLLRVIDEIPGGKIFGAIWPLALGALKNLIK (SEQ ID NO:296).

### Embodiment 74

The construct of embodiment 68, wherein the amino acid sequence of said construct consists of the amino acid sequence LATKLKYEKEHKKMGGGGFLKFLKKFFKKLKYY (SEQ ID NO:297).

### Embodiment 75

The construct of embodiment 68, wherein the amino acid sequence of said construct consists of the amino acid sequence LATKLKYEKEHKKMGKIFGAIWPLALGALKNLIK (SEQ ID NO:298).

### Embodiment 76

The construct of embodiment 68, wherein the amino acid sequence of said construct consists of the amino acid sequence LATLKKYLKEHKKMGKIFGAIWPLALGALKNLIK (SEQ ID NO:299).

### Embodiment 77

The construct of embodiment 68, wherein the amino acid sequence of said construct consists of the amino acid sequence NILRVLKQVWKGGGKNLRIIRKGIHIIKKY (SEQ ID NO:300).

### Embodiment 78

The construct of embodiment 68, wherein the amino acid sequence of said construct consists of the amino acid sequence GNFYRLFKDILKGGGKNLRIIRKGIHIIKKY (SEQ ID NO:301).

### Embodiment 79

The construct according to any one of embodiments 41-78, wherein said construct further comprises a chemoattractant peptide sequence.

### Embodiment 80

The construct of embodiment 79, wherein said chemoattractant peptide sequence is attached to the carboxyl terminal of said construct.

### Embodiment 81

The construct of embodiment 79, wherein said chemoattractant peptide sequence is attached to the amino terminal of said construct.

### Embodiment 82

The construct of embodiment 79, wherein said chemoattractant peptide sequence is between the targeting peptide and the effector.

### Embodiment 83

The construct according to any one of embodiments 79-82, wherein the amino acid sequence of said chemoattractant peptide comprises or consists of the motif XKYX(P/V)M (SEQ ID NO:289) where X is any amino acid and M is methionine or D-methionine.

### Embodiment 84

The construct of embodiment 83, wherein the amino acid sequence of said chemoattractant peptide comprises or consists of the amino acid sequence WKYMVM (SEQ ID NO:290) where sequence consists of all “L” amino acids.

### Embodiment 85

The construct of embodiment 83, wherein the amino acid sequence of said chemoattractant peptide comprises or consists of the amino acid sequence WKYMVM (SEQ ID NO:290) where sequence consists of all “D” amino acids.

### Embodiment 86

The construct of embodiment 83, wherein the amino acid sequence of said chemoattractant peptide comprises or consists of the amino acid sequence WKYMV(dM) (SEQ ID NO: 291) where dM is D-methionine and the other resides are all L residues.

### Embodiment 87

The construct according to any one of embodiments 41-86, wherein said construct bears no terminal protecting groups.

### Embodiment 88

The construct according to any one of embodiments 41-86, wherein said construct bears one or more protecting groups.

### Embodiment 89

The construct of embodiment 88, wherein said one or more protecting groups are independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), and Trifluoroacetyl (TFA).

### Embodiment 90

The construct of embodiments 88 or 89, wherein construct comprises a protecting group at a carboxyl and/or amino terminus.

### Embodiment 91

The construct of embodiment 90, wherein a carboxyl terminus is amidated.

### Embodiment 92

The construct of embodiments 90 or 91, wherein an amino terminus is acetylated.

### Embodiment 93

The construct according to any one of embodiments 41-92, wherein said construct is functionalized with a polymer to increase serum half-life.

### Embodiment 94

The construct of embodiment 93, wherein said polymer comprises polyethylene glycol and/or a cellulose or modified cellulose.

### Embodiment 95

An antimicrobial peptide (AMP) comprising or consisting of the amino acid sequence FLKFLKKFFKKLKYY (SEQ ID NO:42) or a truncated version thereof that retains antimicrobial activity.

### Embodiment 96

The antimicrobial peptide of embodiment 95, wherein said AMP comprises or consists of the amino acid sequence FLKFLKKFFKKLKYY (SEQ ID NO:42).

### Embodiment 97

The antimicrobial peptide according to any one of embodiments 95-96, wherein said AMP bears one or more protecting groups.

### Embodiment 98

The antimicrobial peptide of embodiment 97, wherein said one or more protecting groups are independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), and Trifluoroacetyl (TFA).

### Embodiment 99

The AMP of embodiments 97 or 98, wherein construct comprises a protecting group at a carboxyl and/or amino terminus.

### Embodiment 100

The AMP of embodiment 99, wherein a carboxyl terminus is amidated.

### Embodiment 101

The AMP of embodiments 99 or 100, wherein an amino terminus is acetylated.

### Embodiment 102

A pharmaceutical composition comprising a construct according to any of embodiments 41-94, and/or an AMP according to any one of embodiments 95-101, in a pharmaceutically acceptable carrier.

### Embodiment 103

The pharmaceutical composition of embodiment 102, wherein said composition is formulated as a unit dosage formulation.

### Embodiment 104

The pharmaceutical composition of embodiment 102, wherein said composition is formulated for administration by a modality selected from the group consisting of intraperitoneal administration, topical administration, oral administration, inhalation administration, transdermal administration, subdermal depot administration, ocular administration, and rectal administration.

### Embodiment 105

The pharmaceutical composition of embodiment 102, wherein said composition is formulated for specific or preferential delivery to the colon, esophagus, stomach, large intestine, or small intestine in a mammal.

### Embodiment 106

The pharmaceutical composition of embodiment 105, wherein said composition is formulated for specific or preferential delivery to the colon in a mammal using a formulation form selected from the group consisting of pH sensitive coating, delayed time-controlled release system, microbial triggered drug delivery, pressure controlled drug delivery, Colon Targeted Delivery System (CODES™), and Osmotic Controlled Drug Delivery (ORDS-CT).

### Embodiment 107

The pharmaceutical composition of embodiment 106, wherein said composition is formulated with a pH sensitive coating based on different classes of polymers such as polyacrylates, polymethacrylates, hypromellose acetate succinate, hypromellose phthalate and the like.

### Embodiment 108

The pharmaceutical composition of embodiment 106, wherein said composition is formulated with a delayed time-controlled release system. Examples include, but are not limited to water soluble, erodible polymer based coatings based on pharmaceutically acceptable polysaccharides such as hydroxypropyl or hydroxyethyl cellulose and the like, and/or water insoluble, but capable of swelling (swellable), polymer coatings based on polymethacrylates (e.g., copolymers of ethyl acrylate, methyl methacrylate and a low content of methacrylic acid ester with quaternary ammonium groups (such as EUDRAGIT®RS, EUDRAGIT®RL (see, e.g., Evonic Industries) and the like) that delay drug release through, e.g. diffusional constrains, and the like.

### Embodiment 109

The pharmaceutical composition of embodiments 105 or 106, wherein said composition comprises a capsule filled with or tablet comprising a construct according to any of embodiments 41-94, and/or an AMP according to any one of embodiments 95-101, pharmaceutically acceptable water soluble matrix or filler/osmotic agent such as mannitol or equivalent pharmaceutical excipient, and water swellable polymer such as hypromellose or the like.

### Embodiment 110

The pharmaceutical composition of embodiment 109, wherein the construct or AMP component is preformulated with a pharmaceutically acceptable buffer, bulking agent and/or lyoprotectant (e.g., mannitol and/or trehalose or other equivalent excipient), lyophilized, and processed by milling and sieving into lyo-powder.

### Embodiment 111

The pharmaceutical composition of embodiment 109, wherein the construct or AMP component is preformulated with a pharmaceutically acceptable buffer, bulking agent and/or lyoprotectant (e.g., mannitol and/or trehalose or other equivalent excipient), and spray drying processing is used for re-formulated API powder preparation/manufacturing.

### Embodiment 112

The composition of embodiment 111, wherein said powder is a dry powder having a mean particle size ranging from about 0.1 μm to about 50 μm, or from 0.5 μm to about 25 μm.

### Embodiment 113

The pharmaceutical composition of embodiment 106, wherein said composition is formulated using a microbial triggered drug delivery system.

### Embodiment 114

The pharmaceutical composition of embodiment 113, wherein said composition is formulated using a microbial-triggered drug delivery system that comprises a prodrug formulation.

### Embodiment 115

The pharmaceutical composition of embodiment 113, wherein said composition is formulated as a prodrug selected from the group consisting of an azo conjugate, a saccharide conjugate or carrier, a hydrophobic amino acid conjugate, a sulphapyridine conjugate, and a glucouronic acid conjugate.

### Embodiment 116

The pharmaceutical composition of embodiment 113, wherein said composition is formulated using a polysaccharide delivery system.

### Embodiment 117

The pharmaceutical composition of embodiment 116 wherein said polysaccharide delivery system comprises a polysaccharide selected from the group consisting of chitosan, a chitosan derivative (e.g., chitosan succinate), pectin, a pectin derivative (e.g., amidated pectin, calcium pectinate), chondroitin, and an alginate.

### Embodiment 118

The pharmaceutical composition of embodiment 106, wherein said composition is formulated using a pressure controlled drug delivery system.

### Embodiment 119

The pharmaceutical composition of embodiment 118, wherein said delivery system comprises a colon-delivery capsules comprising ethylcellulose.

### Embodiment 120

The pharmaceutical composition of embodiment 106, wherein said composition is formulated using a Colon Targeted Delivery System (CODES™).

### Embodiment 121

The pharmaceutical composition of embodiment 120, wherein said composition is as core containing lactulose which is overcoated with an acid soluble material.

### Embodiment 122

The pharmaceutical composition of embodiment 106, wherein said composition is formulated using an Osmotic Controlled Drug Delivery (ORDS-CT) system.

### Embodiment 123

The pharmaceutical composition of embodiment 122, wherein said composition is formulated using bilayer push pull unit(s) containing an osmotic push layer and a drug layer, both surrounded by a semipermeable membrane.

### Embodiment 124

A method of killing or inhibiting the growth or proliferation of Clostridium difficile, said method comprising:
contacting said C. difficile with a composition comprising a construct according to any one of embodiments 41-94 and/or administering a pharmaceutical formulation according to any one of embodiments 102-123, in an amount effective to kill or inhibit the growth or proliferation of C. difficile.

### Embodiment 125

The method of embodiment 124, wherein said method comprises contacting said C. difficile with a composition comprising a construct according to any one of embodiments 41-94.

### Embodiment 126

The method of embodiment 124, wherein said method comprises administering a pharmaceutical formulation according to any one of embodiments 102-123.

### Embodiment 127

The method according to any one of embodiments 124-126, wherein said contacting or administering comprises administering said construct or said pharmaceutical formulation to a mammal determined to have a C. difficile infection.

### Embodiment 128

The method according to any one of embodiments 124-126, wherein said contacting or administering comprises prophylactic administration of said construct or said pharmaceutical formulation to a mammal to prevent or to reduce the severity of a C. difficile infection.

### Embodiment 129

The method according to any one of embodiments 127-128, wherein said mammal is a non-human mammal.

### Embodiment 130

The method according to any one of embodiments 127-128, wherein said mammal is a human.

### Embodiment 131

A composition comprising fecal matter for fecal transplantation, said composition comprising fecal matter combined with a construct according to any one of embodiments 41-94.

### Embodiment 132

The composition of embodiment 131, wherein viable Clostridium difficile in said fecal matter is preferentially reduced as compared to other bacteria in said fecal matter.

### Embodiment 133

The composition according to any one of embodiments 131-132, wherein said fecal matter comprises human stool.

### Embodiment 134

The composition according to any one of embodiments 131-132, wherein said fecal matter comprises stool from a non-human mammal.

### Embodiment 135

The composition of embodiment 134 wherein said fecal matter comprises stool from a non-human mammal selected from the group consisting of a feline, a canine, a porcine, a bovine, an equine, and a non-human primate.

### Embodiment 136

A method of preparing material for fecal transplantation, said method comprising combining material comprising stool from a mammal with a construct according to any one of embodiments 41-94 in an amount sufficient to preferentially reduce or eliminate viable C. difficile in said stool.

### Embodiment 137

A method of treating a gasteroenterologic disease, in a subject in need thereof, said method comprising performing a fecal matter transplant into said subject using a composition according to any one of embodiments 131-135.

### Embodiment 138

The method of embodiment 137, wherein said gasteroenterologic disease is selected from the group consisting of a C. difficile infection, ulcerative colitis, Crohn's disease, irritable bowel syndrome, and idiopathic constipation.

### Embodiment 139

The method according of embodiment 138, wherein said gasteroenterologic disease comprises a C. difficile infection.

### Embodiment 140

The method according to any one of embodiments 137-139 wherein said subject is a subject under transitory or chronic treatment with one or more antibiotics.

### Embodiment 141

The method according to any one of embodiments 139-140, wherein said subject is a subject that has had at least three recurrences of C. difficile infection.

### Embodiment 142

The method according to any one of embodiments 139-141, wherein said subject is severely ill because of C. difficile infection.

### Embodiment 143

The method according to any one of embodiments 137-142, wherein said subject has failed antibiotic therapies.

### Embodiment 144

The method of embodiment 143, wherein said subject has failed a pulsed tapered regimen of vancomycin.

### Embodiment 145

The method according to any one of embodiments 137-144, wherein said subject is a human.

### Embodiment 146

The method according to any one of embodiments 137-144, wherein said subject is a non-human mammal.

### Embodiment 147

The method of embodiment 146, wherein said subject is a non-human mammal selected from the group consisting of a feline, a canine, a porcine, a bovine, an equine, and a non-human primate.

### Embodiment 148

The method according to any one of embodiments 137-147, wherein said subject is coadministered an antibiotic.

### Embodiment 149

The method of embodiment 148, wherein said subject is coadministered vancomycin.

### Embodiment 150

The method according to any one of embodiments 137-149, wherein said fecal matter transplantation is performed by introducing the fecal matter in an enema.

### Embodiment 151

The method according to any one of embodiments 137-149, wherein said fecal matter transplantation is performed by introducing the fecal matter in a colonoscopy.

### Embodiment 152

The method according to any one of embodiments 137-149, wherein said fecal matter transplantation is performed by orally administering the fecal matter in capsules.

### Embodiment 153

The method according to any one of embodiments 137-149, wherein said fecal matter transplantation is performed by administering the fecal matter as rectal suppositories.

### DEFINITIONS

The term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide or the peptide can comprise all “D” amino acids. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, phosphoramide, a sulfonamide, a carbonate or carbomate, a hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like).

The term “residue”” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, L-2-amino-3-guanidinopropionic acid (AGP), L-α,γ-diaminobutyric acid (DAB), L-α,β-diaminoproprionic acid (DAP), L-α-t-butylglycine and the like. These modified amino acids are illustrative and not intended to be limiting.

The term “STAMP” refers to Specifically Targeted Anti-Microbial Peptides. In various embodiments, a STAMP comprises one or more targeting peptides (e.g., peptides that bind preferentially or specifically to C. difficile) attached to one or more antimicrobial moieties (e.g., antimicrobial peptides (AMPs)). An MH-STAMP is a STAMP bearing two or more targeting domains (i.e., a multi-headed STAMP).

“β-peptides” contain one or more or comprises all “β amino acids”, which have their amino group bonded to the (3 carbon rather than the α-carbon as in the 20 standard biological amino acids. The only commonly naturally occurring β amino acid is β-alanine.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids).

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr.

Also contemplated herein are non-natural compounds that correspond to the peptides described herein comprising all naturally-occuring amino acids. A compound “corresponds” to a natural peptide if it elicits a biological activity (e.g., antimicrobial activity and/or C. difficile binding) related to the biological activity and/or specificity of the “conventional” peptide. The elicited activity may be the same as, greater than or less than that of the natural peptide.

In certain embodiments non-natural compounds contemplated herein comprises peptoids. A peptoid has an essentially corresponding monomer sequence as the “conventional” peptide, where a natural amino acid is replaced by an N-substituted glycine derivative, if the N-substituted glycine derivative resembles the original amino acid in hydrophilicity, hydrophobicity, polarity, etc. The following are illustrative, but non-limiting N-substituted glycine replacements: N-(1-methylprop-1-yl)glycine→isoleucine (I), N-(prop-2-yl)glycine→valine (V), N-benzylglycine→phenylalanine (F), N-(2-hydroxyethyl)glycine→serine (S), and the like. In certain aspects of the invention, substitutions need not be “exact”. Thus for example, in certain aspects of the invention, N-(2-hydroxyethyl)glycine may substitute for S, T, C, and/or M; N-(2-methylprop-1-yl)glycine may substitute for V, L, and/or I; N-(2-hydroxyethyl)glycine can be used to substitute for T or S. In general, one may use an N-hydroxyalkyl-substituted glycine to substitute for any polar amino acid, an N-benzyl- or N-aralkyl-substituted glycine to replace any aromatic amino acid, an N-alkyl-substituted glycine such as N-butylglycine to replace any nonpolar amino acid (e.g., L, V, I, etc.), and an N-(aminoalkyl)glycine to replace any basic polar amino acid (e.g., K and R).

Where an amino acid sequence is provided herein, L-, D-, or beta amino acid versions of the sequence are also contemplated as well as retro, inversion (inverso), and retro-inversion (retroinverso) isoforms. In addition, conservative substitutions (e.g., in the targeting peptide, and/or antimicrobial peptide, and/or linker peptide (when present)) are contemplated. Non-protein backbones, such as PEG, alkane, ethylene bridged, ester backbones, and other backbones are also contemplated. Also fragments ranging in length from about 5, 6, 7, 8, 9, 10, 11, 12, 13, 14, 15, 16, 17, 18, 19, 20, 21, 22, 23, 24, or 25 amino acids up to the full length minus one amino acid of the peptide are contemplated where the fragment retains at least 50%, preferably at least 60% 70% or 80%, more preferably at least 90%, 95%, 98%, 99%, or at least 100% of the activity (e.g., binding specificity and/or avidity, antimicrobial activity, etc.) of the full length peptide are contemplated.

In certain embodiments, conservative substitutions of the amino acids comprising any of the sequences described herein are contemplated. In various embodiments one, two, three, four, or five different residues are substituted. The term “conservative substitution” is used to reflect amino acid substitutions that do not substantially alter the activity (e.g., antimicrobial activity and/or specificity) of the molecule. Typically conservative amino acid substitutions involve substitution one amino acid for another amino acid with similar chemical properties (e.g. charge or hydrophobicity). Certain conservative substitutions include “analog substitutions” where a standard amino acid is replaced by a non-standard (e.g., rare, synthetic, etc.) amino acid differing minimally from the parental residue. Amino acid analogs are considered to be derived synthetically from the standard amino acids without sufficient change to the structure of the parent, are isomers, or are metabolite precursors. Examples of such “analog substitutions” include, but are not limited to, 1) Lys-Orn, 2) Leu-Norleucine, 3) Lys-Lys[TFA], 4) Phe-Phe[Gly], and 5) δ-amino butylglycine-ξ-amino hexylglycine, where Phe[gly] refers to phenylglycine (a Phe derivative with a H rather than CH₃ component in the R group), and Lys[TFA] refers to a Lys where a negatively charged ion (e.g., TFA) is attached to the amine R group. Other conservative substitutions include “functional substitutions” where the general chemistries of the two residues are similar, and can be sufficient to mimic or partially recover the function of the native peptide. Strong functional substitutions include, but are not limited to 1) Gly/Ala, 2) Arg/Lys, 3) Ser/Tyr/Thr, 4) Leu/Ile/Val, 5) Asp/Glu, 6) Gln/Asn, and 7) Phe/Trp/Tyr, while other functional substitutions include, but are not limited to 8) Gly/Ala/Pro, 9) Tyr/His, 10) Arg/Lys/His, 11) Ser/Thr/Cys, 12) Leu/Ile/Val/Met, and 13) Met/Lys (special case under hydrophobic conditions). Various “broad conservative substations” include substitutions where amino acids replace other amino acids from the same biochemical or biophysical grouping. This is similarity at a basic level and stems from efforts to classify the original 20 natural amino acids. Such substitutions include 1) nonpolar side chains: Gly/Ala/Val/Leu/Ile/Met/Pro/Phe/Trp, and/or 2) uncharged polar side chains Ser/Thr/Asn/Gln/Tyr/Cys. In certain embodiments broad-level substitutions can also occur as paired substitutions. For example, Any hydrophilic neutral pair [Ser, Thr, Gln, Asn, Tyr, Cys]+[Ser, Thr, Gln, Asn, Tyr, Cys] can may be replaced by a charge-neutral charged pair [Arg, Lys, His]+[Asp, Glu]. The following six groups each contain amino acids that, in certain embodiments, are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V); and 6) Phenylalanine (F), Tyrosine (Y), Tryptophan (W). Where amino acid sequences are disclosed herein, amino acid sequences comprising, one or more of the above-identified conservative substitutions are also contemplated.

In certain embodiments, targeting peptides, antimicrobial peptides, and/or STAMPs compromising at least 80%, preferably at least 85% or 90%, and more preferably at least 95% or 98% sequence identity with any of the sequences described herein are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides described herein, in typical embodiments, sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci., USA, 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

The term “specificity” when used with respect to the antimicrobial activity of a peptide indicates that the peptide preferentially binds to and/or inhibits growth and/or proliferation and/or kills a particular microbial species as compared to other related and/or unrelated microbes. In certain embodiments the preferential inhibition or killing is at least 10% greater (e.g., LD₅₀ is 10% lower), preferably at least 20%, 30%, 40%, or 50%, more preferably at least 2-fold, at least 5-fold, or at least 10-fold greater for the target species.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “consisting essentially of” when used with respect to an antimicrobial peptide (AMP) or STAMP as described herein indicates that the peptide, or variants, analogues, or derivatives thereof possess substantially the same or greater antimicrobial activity and/or specificity as the referenced peptide or STAMP. In certain embodiments substantially the same or greater antimicrobial activity indicates at least 80%, preferably at least 90%, more preferably at least 95%, and most preferably at least 98%, or at least 99%, or at least 100% of the antimicrobial activity of the referenced peptide(s) or STAMPs against a particular bacterial species (e.g., Clostridium difficile).

The terms “isolated” “purified” or “biologically pure” refer to material that is substantially or essentially free from components that normally accompany it as found in its native state. In the case of a peptide, an isolated (naturally occurring) peptide is typically substantially free of components with which it is associated in the cell, tissue, or organism. The term isolated also indicates that the peptide is not present in a phage display, yeast display, or other peptide library.

In various embodiments the amino acid abbreviations shown in Table 1 are used herein.

TABLE 1
Amino acid abbreviations.

| | Abbreviation | |
|---|---|---|
| Name | 3 Letter | 1 Letter |
| Alanine | Ala | A |
| βAlanine (NH₂—CH₂—CH₂—COOH) | βAla | |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine methylsulfonium | Met (S—Me) | — |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| episilon-aminocaproic acid | Ahx | J |
| (NH²—(CH₂)₅—COOH) | | |
| 4-aminobutanoic acid | γAbu | |
| (NH₂—(CH₂)₃—COOH) | | |
| tetrahydroisoquinoline-3 | | O |
| carboxylic acid | | |
| Lys(N(epsilon)-trifluoroacetyl) | | K[TFA] |
| α-aminoisobutyric acid | Aib | B |

### BRIEF DESCRIPTION OF THE DRAWINGS

FIG. 1 shows some illustrative porphyrins (compounds 92-99) suitable for use as targeting moieties and/or antimicrobial effectors.

FIG. 2 shows some illustrative porphyrins (compounds 100-118) suitable for use as targeting moieties and/or antimicrobial effectors.

FIG. 3 shows some illustrative porphyrins (in particular phthalocyanines) (compounds 119-128) suitable for use as targeting moieties and/or antimicrobial effectors.

FIG. 4 illustrates the structures of two phthalocyanines, Monoastral Fast Blue B and Monoastral Fast Blue G suitable for use as targeting moieties and/or antimicrobial effectors.

FIG. 5 illustrates certain azine photosensitizers suitable for use as targeting moieties and/or antimicrobial effectors in the compositions and methods described herein.

FIG. 6 shows illustrative cyanine suitable for use as targeting moieties and/or antimicrobial effectors in the compositions and methods described herein.

FIG. 7 shows illustrative psoralen (angelicin) photosensitizers suitable for use as targeting moieties and/or antimicrobial effectors in the compositions and methods described herein.

FIG. 8 shows illustrative hypericin and the perylenequinonoid pigments suitable for use as targeting moieties and/or antimicrobial effectors in the compositions and methods described herein.

FIG. 9 shows illustrative acridines suitable for use as targeting moieties and/or antimicrobial effectors in the compositions and methods described herein.

FIG. 10 illustrates the structure of the acridine Rose Bengal.

FIG. 11 illustrates various crown ethers suitable for use as targeting moieties and/or antimicrobial effectors in the compositions and methods described herein.

FIG. 12 illustrates the structure of cumin.

FIG. 13 illustrates an example of a targeted light-activated porphyrin comprising a porphyrin coupled to a CD0714 (SEQ ID NO:1) C. difficile targeting sequence.

FIG. 14 schematically shows some illustrative configurations for chimeric constructs described herein. A: Shows a single targeting moiety T1 attached to a single effector E1 by a linker/spacer L. B: Shows multiple targeting moieties T1, T2, T3 attached directly to each other and attached by a linker L to a single effector E1. In various embodiments T1, T2, and T3, can be domains in a fusion protein. C: Shows multiple targeting moieties T1, T2, T3 attached to each other by linkers L and attached by a linker L to a single effector E1. In various embodiments T1, T2, and T3, can be domains in a fusion protein. D: Shows a single targeting moiety T1 attached by a linker L to multiple effectors E1, E2, and E3 joined directly to each other. E: Shows a single targeting moiety T1 attached by a linker L to multiple effectors E1, E2, and E3 joined to each other by linkers L. F: Shows multiple targeting moieties joined directly to each other and by a linker L to multiple effectors joined to each other by linkers L. G: Shows multiple targeting moieties joined to each other by linkers L and by a linker L to multiple effectors joined to each other by linkers L. In various embodiments T1, T2, and T3, and/or E1, E2, and E3 can be domains in a fusion protein. H: Illustrates a branched configuration where multiple targeting moieties are linked to a single effector. I: Illustrates a dual branched configuration where multiple targeting moieties are linked to multiple effectors. J: Illustrates a branched configuration where multiple targeting moieties are linked to multiple effectors where the effectors are joined to each other in a linear configuration.

FIG. 15 shows the activity of CD0714-based STAMPs on C. difficile isolate 1803.

FIG. 16 shows the activity of CD0714-based STAMPs on L. casei.

FIG. 17 shows the activity of CD0714-based STAMPs on B. fragilis.

FIG. 18 shows the activity of CD0126-based STAMPs on C. difficile isolate 1803.

FIG. 19 shows the activity of CD0126-based STAMPs on L. casei.

FIG. 20 shows the activity of CD0126-based STAMPs on B. fragilis.

FIG. 21 shows the activity of CD9232/CD8040-based STAMPs on C. difficile isolate 1803.

FIG. 22 shows the activity of CD9232/CD8040-based STAMPs on L. casei.

FIG. 23 shows the activity of CD9232/CD8040-based STAMPs on B. fragilis.

### DETAILED DESCRIPTION

In various embodiments targeting peptides are provided that bind (e.g., that preferentially and/or specifically bind to Clostridium difficile. One or more such targeting peptides can be attached to one or more “effector moieties” (e.g., a detectable label, a porphyrin or other photosensitizer, an antimicrobial peptide, an antibiotic, a ligand, a lipid or liposome, a polymeric particle, etc.) to provide constructs that are capable of delivering the effector(s) to a target (e.g., C. difficile) in vivo or ex vivo. In certain embodiments, the targeting peptide(s) are attached (directly or through a linker (e.g., an amino acid, a peptide linker, a heterobifunctional linker, etc.)) to one or more antimicrobial peptide(s) (AMP(s)) thereby affording specificity/selectivity to the antimicrobial peptide. Such constructs may be designated as specifically-targeted antimicrobial peptides or “STAMPs”.

Illustrative targeting peptides that bind C. difficile are shown in Table 2.

TABLE 2
Illustrative peptides that bind to (target)
C. difficile.

| | | SEQ ID |
|---|---|---|
| Family/Peptide | Sequence | NO: |
| CD0714 Family: | | |
| PF-285 | VPAKLLRVIDEIPE | 2 |
| CD0714 | VPAKLLRVIDEIP | 3 |
| CD0714 | VPAKLLRVIKKIP | 4 |
| | VPAKLLRVIKEIP | 5 |
| | VPAKLLRVIKKIPE | 6 |
| retro PF-285 | EPIEDIVRLLKAPV | 7 |
| retro CD0714 | PIEDIVRLLKAPV | 8 |
| retro CD0714 | EPIKKIVRLLKAPV | 9 |
| retro | PIKKIVRLLKAPV | 10 |
| CD0126 Family | | |
| CD0126 (PF-299) | LATKLKYEKEHKKM | 11 |
| | LATLKKYLKEHKKM | 12 |
| | ATKLKYEKEHKKM | 13 |
| | LATKLKYEKEHKK | 14 |
| | ATKLKYEKEHKK | 15 |
| retro CD0126 (PF-299) | MKKHEKEYKLKTAL | 16 |
| retro | MKKHEKEYKLKTA | 17 |
| retro | KKHEKEYKLKTAL | 18 |
| retro | KKHEKEYKLKTA | 19 |
| Genomic family | | |
| | NILRVLKQVWK | 20 |
| | ILRVLKQVWK | 21 |
| | NILRVLKQVW | 22 |
| | ILRVLKQVW | 23 |
| retro | KWVQKLVRLIN | 24 |
| retro | WVQKLVRLIN | 25 |
| retro | KWVQKLVRLI | 26 |
| retro | WVQKLVRLI | 27 |
| | GNFYRLFKDILK | 28 |
| | NFYRLFKDILK | 29 |
| | GNFYRLFKDIL | 30 |
| | NFYRLFKDIL | 31 |
| retro | KLIDKFLRYFNG | 32 |
| retro | LIDKFLRYFNG | 33 |
| retro | KLIDKFLRYFN | 34 |
| retro | LIDKFLRYFN | 35 |

In certain embodiments the targeting peptide may comprise any one or more of the amino acids sequences shown in Table 2. In certain embodiments the targeting peptide(s) may range in length up to about 100 amino acids or up to about 80 amino acids, or up to about 60 amino acids, or up to about 50 amino acids, or up to about 45 amino acids, or up to about 40 amino acids, or up to about 35 amino acids, or up to about 30 amino acids, or up to about 25 amino acids, or up to about 20 amino acids, or up to about 15 amino acids. In certain embodiments the targeting peptide comprises one copy of an amino acid sequence shown in Table 2. In certain embodiments the targeting peptide comprises at least two copies, or at least 3 copies, or at least 4 copies, or at least 5 copies of a targeting peptide sequence shown in Table 2. In certain embodiments the targeting peptide comprises at least two different sequence, or at least 3 different sequences, or at least 4 different sequences, or at least 5 different amino acid sequences shown in Table 2. In certain embodiments the targeting peptide consists of one of the amino acid sequences shown in Table 2.

In certain embodiments any of the amino acid sequences shown in Table 2 can comprise at least one beta amino acid, or at least one “D” amino acid, or at least 2 beta amino acids, or at least two “D” amino acids, or at least 3 beta amino acids, or at least 3″D″ amino acids, or at least 4 beta amino acids, or at least 4 “D” amino acids, or at least 5 beta amino acids, or at least 5 “D” amino acids, or at least 6 beta amino acids, or at least 6 “D” amino acids, or at least 7 beta amino acids, or at least 7 “D” amino acids, or at least 8 beta amino acids, or at least 8 “D” amino acids, or at least 9 beta amino acids, or at least 9 “D” amino acids, or at least 10 beta amino acids, or at least 10 “D” amino acids. In certain embodiments any of the amino acid sequences shown in Table 2 comprise all beta amino acids or all “D” amino acids.

In certain embodiments any of the foregoing peptides can comprise at least one conservative substitution, or at least two conservative substitutions, or at least 3 conservative substitutions, or at least 4 conservative substitutions, or at least 5 conservative substitutions. In certain embodiments any of the foregoing peptides can comprise at least 1 internal deletion, at least 2 internal deflections, at least 3 internal deletions, or at least 4 internal deletions. In certain embodiments any of the foregoing peptides can comprise at least 1 carboxyl and/or amino terminal truncation, or at least 2 carboxyl and/or amino terminal truncations, or at least 3 carboxyl and/or amino terminal truncations, or at least 4 carboxyl and/or amino terminal truncations, or at least 5 carboxyl and/or amino terminal truncations.

The foregoing C. difficile targeting peptides are illustrative and non-limiting. Using the teaching provided herein, numerous other C. difficile targeting peptides will be available to one of skill in the art.

### Design and Construction of STAMPs and Other Chimeric Constructs.

In various embodiments, one or more C. difficile targeting peptides described herein can be attached to one or more effectors (e.g., an antimicrobial peptide, an antibiotic, a ligand, a lipid or liposome (e.g., a lipid or liposome containing a drug), a detectable label, a porphyrin, a photosentizing agent, an epitope tag, etc.) to form a chimeric constructs.

The effector typically comprises one or more moieties whose activity is to be delivered to the target microorganism(s) (e.g., C. difficile), to an organ, cell, or tissue containing the target microorganism(s), and the like.

In certain embodiments one or more targeting peptides are attached to a single effector. In certain embodiments one or more effectors are attached to a single targeting peptide. In certain embodiments multiple targeting peptides are attached to multiple effectors. The targeting moieties(s) can be attached directly to the effector(s) or through a linker. Where both the targeting peptide and the effector comprise peptides the chimeric moiety (e.g., a STAMP) can be a fusion protein.

Effector Moieties that can be Attached to C. difficile Targeting Peptides.

Any of a wide number of effectors can be coupled to targeting peptides as described herein to preferentially deliver the effector to a target organism (e.g., C. difficile) and/or tissue. Illustrative effectors include, but are not limited to detectable labels, small molecule antibiotics, antimicrobial peptides, porphyrins or other photosensitizers, epitope tags/antibodies for use in a pretargeting protocol, agents that physically disrupt the extracellular matrix within a community of microorganisms, microparticles and/or microcapsules, nanoparticles and/or nanocapsules, “carrier” vehicles including, but not limited to lipids, liposomes, dendrimers, cholic acid-based peptide mimics or other peptide mimics, steroid antibiotics, and the like.

Antimicrobial Peptides.

In certain embodiments, the C. difficile targeting peptides described herein (e.g., peptides shown in Table 2) can be attached to one or more antimicrobial peptides to form selectively targeted antimicrobial peptides (STAMPs) that are effective to kill and/or to inhibit the growth and/or proliferation of C. difficile. Moreover, it is believed that such STAMPs will preferentially or specifically inhibit C. difficile when present in a mixed bacterial population such as is found in the gut.

In certain embodiments the antimicrobial peptides comprise one or more amino acid sequences described for example below in Table 3). In certain embodiments the antimicrobial peptides comprise one or more amino acid sequences described in the “Collection of Anti-Microbial Peptides” (CAMP) an online database developed for advancement the understanding of antimicrobial peptides (see, e.g., Thomas et al. (2009) Nucleic Acids Research, 2009, 1-7. doi:10.1093/nar/gkp1021) available at www.bicnirrh.res.in/antimicrobial and/or one or more AMP amino acid sequences described in PCT Patent Publication No: PCT/US2010/020242 (WO 2010/080819), which is incorporated herein by reference for the antimicrobial peptides described therein.

TABLE 3
Illustrative, but non-limiting list of antimicrobial peptides suitable for use in
constructs (e.g., STAMPs) comprising the Clostridium difficile targeting peptides described
herein.

| | | | SEQ |
|---|---|---|---|
| ID | Organism MIC | Sequence | ID NO |
| | | FIGAIARLLSKIFGKR | 36 |
| | | GIFSKLAGKKIKNLLISG | 37 |
| | | GIFSKLAGKKIKNLLISGLKG | 38 |
| | | GLFSKFVGKGIKNFLIKGVK | 39 |
| | | KAYSTPRCKGLFRALMCWL | 40 |
| | | KIFGAIWPLALGALKNLIK | 41 |
| | | FLKFLKKFFKKLKYY | 42 |
| | | GWGSFFKKAAHVGKHVGKAALTHY | 43 |
| | | L | |
| | | RGLRRLGRKIAHGVKKYG | 44 |
| | | RGLRRLGRKIAHGVKKYGPTVLRI | 45 |
| | | IRIAG | |
| | | KIAHGVKKYGPTVLRIIR | 46 |
| | | LLGDFFRKSKEKIGKEFKRIVQRI | 47 |
| | | KDFLRNLVPRTES | |
| | | FLPLIGRVLSGIL | 48 |
| | | IGKFLKKAKKFGKAFVKILKK | 49 |
| | | GKFLKKAKKFGKAFVKIL | 50 |
| | | WFLKFLKKFFKKLKY | 51 |
| | | WFLKFLKKFFKKLK | 52 |
| | | RGLRRLGRKIAHGVKKY | 53 |
| | | LLGDFFRKSKEKI | 54 |
| | | ILRWPWWPWRRK | 55 |
| 1C-1 | | RRRRWWW | 56 |
| 1C-2 | | RRWWRRW | 57 |
| 1C-3 | | RRRWWWR | 58 |
| 1C-4 | | RWRWRWR | 59 |
| 1T-88 | | GRLVLEITADEVKALGEALANAKI | 60 |
| 1T-88 | | GRLVLEITADEVKALGEALANAKI | 61 |
| 2C-1 | | RRRFWWR | 62 |
| 2C-2 | | RRWWRRF* | 63 |
| 2C-3 | | RRRWWWF* | 64 |
| 2C-4 | | RWRWRWF* | 65 |
| 3C-1 | | RRRRWWK | 66 |
| 3C-2 | | RRWWRRK | 67 |
| 3C-3 | | RRRWWWK | 68 |
| 3C-4 | | RWRWRWK | 69 |
| 4C-1 | | RRRKWWK | 70 |
| 4C-2 | | RRWKRRK | 71 |
| 4C-3 | | RRRKWWK | 72 |
| 4C-4 | | RWRKRWK | 73 |
| a-10 | | KLKKLLKRWRRWWR | 74 |
| a-11 | | RWRRLLKKLHHLLH* | 75 |
| a-12 | | KLKKLLKHLHHLLH* | 76 |
| a-3 | | LHLLHQLLHLLHQF* | 77 |
| a-4 | | AQAAHQAAHAAHQF* | 78 |
| a-5 | | KLKKLLKKLKKLLK | 79 |
| a-6 | | LKLLKKLLKLLKKF* | 80 |
| a-7 | | LQLLKQLLKLLKQF* | 81 |
| a-8 | | AQAAKQAAKAAKQF* | 82 |
| a-9 | | RWRRWWRHFHHFFH* | 83 |
| AA-1 | | HHFFHHFHHFFHHF* | 84 |
| AA-2 | | FHFFHHFFHFFHHF* | 85 |
| AA-3 | | KLLKGATFHFFHHFFHFFHHF | 86 |
| AA-4 | | KLLKFHFFHHFFHFFHHF | 87 |
| AA-5 | | FHFFHHFFHFFHHFKLLK | 88 |
| AF5 | | FLKFLKKFFKKLK | 89 |
| B-33 | | FKKFWKWFRRF | 90 |
| B-34 | | LKRFLKWFKRF | 91 |
| B-35 | | KLFKRWKHLFR | 92 |
| B-36 | | RLLKRFKHLFK | 93 |
| B-37 | | FKTFLKWLHRF | 94 |
| B-38 | | IKQLLHFFQRF | 95 |
| B-39 | | KLLQTFKQIFR | 96 |
| B-40 | | RILKELKNLFK | 97 |
| B-41 | | LKQFVHFIHRF | 98 |
| B-42 | | VKTLLHIFQRF | 99 |
| B-43 | | KLVEQLKEIFR | 100 |
| B-44 | | RVLQEIKQILK | 101 |
| B-45 | | VKNLAELVHRF | 102 |
| B-46 | | ATHLLHALQRF | 103 |
| B-47 | | KLAENVKEILR | 104 |
| B-48 | | RALHEAKEALK | 105 |
| B-49 | | FHYFWHWFHRF | 106 |
| B-50 | | LYHFLHWFQRF | 107 |
| B-51 | | YLFQTWQHLFR | 108 |
| B-52 | | YLLTEFQHLFK | 109 |
| B-53 | | FKTFLQWLHRF | 110 |
| B-54 | | IKTLLHFFQRF | 111 |
| B-55 | | KLLQTFNQIFR | 112 |
| B-56 | | TILQSLKNIFK | 113 |
| B-57 | | LKQFVKFIHRF | 114 |
| B-58 | | VKQLLKIFNRF | 115 |
| B-59 | | KLVQQLKNIFR | 116 |
| B-60 | | RVLNQVKQILK | 117 |
| B-61 | | VKKLAKLVRRF | 118 |
| B-62 | | AKRLLKVLKRF | 119 |
| B-63 | | KLAQKVKRVLR | 120 |
| B-64 | | RALKRIKHVLK | 121 |
| BD-1 | | FVFRHKWVWKHRFLF | 122 |
| BD-10 | | FKAHIRFKLRVKFHF | 123 |
| BD-11 | | LKAKIKFKVKLKIKF | 124 |
| BD-12 | | WIWKHKFLHRHFLF | 125 |
| BD-13 | | VFLHRHVIKHKLVF | 126 |
| BD-14 | | FLHKHVLRHRIVF | 127 |
| BD-15 | | VFKHKIVHRHILF | 128 |
| BD-16 | | FLFKHLFLHRIFF | 129 |
| BD-17 | | LFKHILIHRVIF | 130 |
| BD-18 | | FLHKHLFKHKLF | 131 |
| BD-19 | | VFRHRFIHRHVF | 132 |
| BD-2 | | VFIHRHVWVHKHVLF | 133 |
| BD2.21 | S. mutans, 4 μM | KLFKFLRKHLL | 134 |
| K-10 | | | |
| BD-20 | | FIHKLVHKHVLF | 135 |
| BD-21 | | VLRHLFRHRIVF | 136 |
| BD-22 | | LVHKLILRHLLF | 137 |
| BD-23 | | VFKRVLIHKLIF | 138 |
| BD-24 | | IVRKFLFRHKVF | 139 |
| BD-25 | | VLKHVIAHKRLF | 140 |
| BD-26 | | FIRKFLFKHLF | 141 |
| BD-27 | | VIRHVWVRKLF | 142 |
| BD-28 | | FLFRHRFRHRLVF | 143 |
| BD-29 | | LFLHKHAKHKFLF | 144 |
| BD-3 | | WRWRARWRWRLRWRF | 145 |
| BD-30 | | FKHKFKHKFIF | 146 |
| BD-31 | | LRHRLRHRLIF | 147 |
| BD-32 | | LILKFLFKFVF | 148 |
| BD-33 | | VLIRILVRVIF | 149 |
| BD-34 | | FRHRFRHRF | 150 |
| BD-35 | | LKHKLKHKF | 151 |
| BD-36 | | FKFKHKLIF | 152 |
| BD-37 | | LRLRHRVLF | 153 |
| BD-38 | | FKFLFKFLF | 154 |
| BD-39 | | LRLFLRWLF | 155 |
| BD-4 | | WRIHLRARLHVKFRF | 156 |
| BD-40 | | FKFLFKHKF | 157 |
| BD-41 | | LRLFLRHRF | 158 |
| BD-42 | | FKFLFKF | 159 |
| BD-43 | | LRLFLRF | 160 |
| BD-5 | | LRIHARFKVHIRLKF | 161 |
| BD-6 | | FHIKFRVHLKVRFHF | 162 |
| BD-7 | | FHVKIHFRLHVKFHF | 163 |
| BD-8 | | LHIHAHFHVHIHLHF | 164 |
| BD-9 | | FKIHFRLKVHIRFKF | 165 |
| CAM135 | | GWRLIKKILRVFKGL | 166 |
| Cys-LL-37 | | CLLGDFFRKSKEKIGKEFKRIVQR | 167 |
| | | IKDFLRNLVPRTES | |
| Cys-LL-37-Cys | | CLLGDFFRKSKEKIGKEFKRIVQR | 168 |
| | | IKDFLRNLVPRTESC | |
| G2 | | KNLRIIRKGIHIIKKY | 169 |
| K-1 | S. mutans, 25 μM | GLGRVIGRLIKQIIWRR | 170 |
| K-11 | S. mutans, 4 μM | KILKFLFKQVF | 171 |
| K-12 | S. mutans, 8 μM | KILKKLFKFVF | 172 |
| K-13 | S. mutans, 16 μM | GILKKLFTKVF | 173 |
| K-14 | S. mutans, 8 μM | LRKFLHKLF | 174 |
| K-15 | S. mutans, 4 μM | LRKNLRWLF | 175 |
| K-16 | S. mutans, 8 μM | FIRKFLQKLHL | 176 |
| | P. aeruginosa, 12.5 | | |
| | μM | | |
| | MRSA, 25 μM | | |
| K-17 | S. mutans, 8 μM | FTRKFLKFLHL | 177 |
| K-18 | S. mutans, 16 μM | KKFKKFKVLKIL | 178 |
| K-19 | S. mutans, 16 μM | LLKLLKLKKLKF | 179 |
| K-2 | S. mutans, 12.5 μM | VYRKRKSILKIYAKLKGWH | 180 |
| K-20 (A5 + Y) | S. mutans, 8 μM | FLKFLKKFFKKLKY | 181 |
| K-21 | S. mutans, 8 μM | GWLKMFKKIIGKFGKF | 182 |
| K-22 | S. mutans, 8 μM | GIFKKFVKILYKVQKL | 183 |
| K-7 | S. mutans, 12.5 μM | NYRLVNAIFSKIFKKKFIKF | 184 |
| K-8 | S. mutans, 4 μM | KILKFLFKKVF | 185 |
| K-9 | S. mutans, 4 μM | FIRKFLKKWLL | 186 |
| LL-37 | | LLGDFFRKSKEKIGKEFKRIVQRI | 187 |
| | | KDFLRNLVPRTES | |
| LL-37(17-32) | | FKRIVQRIKDFLRNLV | 188 |
| LL-37-Cys | | LLGDFFRKSKEKIGKEFKRIVQRI | 189 |
| | | KDFLRNLVPRTESC | |
| LL-37FK-13 | | FKRIVQRIKDFLR | 190 |
| LL-37FKR | | FKRIVQRIKDFLRNLVPRTES | 191 |
| LL-37GKE | | GKEFKRIVQRIKDFLRNLVPR | 192 |
| LL-37KRI | | KRIVQRIKDFLRNLVPRTES | 193 |
| LL-37LLG | | LLGDFFRKSKEKIGKEFKRIV | 194 |
| LL-37RKS | | RKSKEKIGKEFKRIVQRIKDFLRN | 195 |
| | | LVPRTES | |
| LL-37SKE | | SKEKIGKEFKRIVQRIKDFLR | 196 |
| Novispirin G10 | | KNLRRIIRKGIHIIKKYG | 197 |
| Novispirin G7 | | KNLRRIGRKIIHIIKKYG | 198 |
| Novispirin T10 | | KNLRRIIRKTIHIIKKYG | 199 |
| Novispirin T7 | | KNLRRITRKIIHIIKKYG | 200 |
| Ovispirin | | KNLRRIIRKIIHIIKKYG | 201 |
| PF-006 | A. baumannii, 50 | MGIIAGIIKFIKGLIEKFTGK | 202 |
| | μM | | |
| | B. subtilis, 25 μM | | |
| | MRSA, 50 μM | | |
| PF-148 | A. niger, 50 μM | RRGCTERLRRMARRNAWDLYAEHF | 203 |
| | B. subtilis, 50 μM | Y | |
| PF-168 | Trubrum, 50 μM | VLPFPAIPLSRRRACVAAPRPRSR | 204 |
| | A. niger, 50 μM | QRAS | |
| | MRSA, 50 μM | | |
| PF-209 | MRSA, 50 μM | NYAVVSHT | 205 |
| PF-278 | C. albicans, 50 μM | LSLATFAKIFMTRSNWSLKRFNRL | 206 |
| | Trubrum, 50 μM | | |
| | S. epidermidis, 50 | | |
| | μM | | |
| PF-283 | Trubrum, 50 μM | MIRIRSPTKKKLNRNSISDWKSNT | 207 |
| | B. subtilis, 50 μM | SGRFFY | |
| | S. epidermidis, 50 | | |
| | μM | | |
| PF-307 | C. albicans, 50 μM | MKRRRCNWCGKLFYLEEKSKEAYC | 208 |
| | Trubrum, 50 μM | CKECRKKAKKVKK | |
| | B. subtilis, 50 μM | | |
| PF-322 | B. subtilis, 50 μM | GIVLIGLKLIPLLANVLR | 209 |
| PF-437 | S. pneumoniae, 50 | FQKPFTGEEVEDFQDDDEIPTII | 210 |
| | μM | | |
| PF-448 | A. niger, 25 μM | SLQSQLGPCLHDQRH | 211 |
| | S. pneumoniae, 50 | | |
| | μM | | |
| PF-497 | B. subtilis, 50 μM | LVLRICTDLFTFIKWTIKQRKS | 212 |
| PF-499 | B. subtilis, 50 μM | VYSFLYVLVIVRKLLSMKKRIERL | 213 |
| PF-511 | S. pneumoniae, 50 | VMQSLYVKPPLILVTKLAQQN | 214 |
| | μM | | |
| PF-512 | S. pneumoniae, 50 | SFMPEIQKNTIPTQMK | 215 |
| | μM | | |
| PF-520 | S. pneumoniae, 50 | LGLTAGVAYAAQPTNQPTNQPTNQ | 216 |
| | μM | PTNQPTNQPTNQPRW | |
| PF-521 | S. pneumoniae, 50 | CGKLLEQKNFFLKTR | 217 |
| | μM | | |
| PF-522 | C. difficile, 25 | FELVDWLETNLGKILKSKSA | 218 |
| PF-523 | S. pneumoniae, 50 | ASKQASKQASKQASKQASKQASRS | 219 |
| | μM | LKNHLL | |
| PF-524 | S. pneumoniae, 50 | PDAPRTCYHKPILAALSRIVVTDR | 220 |
| | μM | | |
| PF-525 | A. niger, 50 μM | KFSDQIDKGQDALKDKLGDL | 221 |
| | S. pneumoniae, 50 | | |
| | μM | | |
| PF-527 | P. aeruginosa, 50 | GSVIKKRRKRMAKKKHRKLLKKTR | 222 |
| | μM | IQRRRAGK | |
| | Trubrum, 25 μM | | |
| | A. niger, 50 μM | | |
| | B. subtilis, 12.5 μM | | |
| | C. jeikeium, 6.25 | | |
| | μM | | |
| | MRSA, 50 μM | | |
| | S. epidermidis, 25 | | |
| | μM | | |
| PF-529 | A. niger, 50 μM | LSEMERRRLRKRA | 223 |
| | S. pneumoniae, 50 | | |
| | μM | | |
| PF-530 | A. baumannii, 25 | SKFKVLRKIIIKEYKGELMLSIQK | 224 |
| | μM | QR | |
| PF-531 | C. difficile, 12.5 | YIQFHLNQQPRPKVKKIKIFL | 225 |
| | μM | | |
| PF-538 | C. difficile, 25 μM | KNKKQTDILEKVKEILDKKKKTKS | 226 |
| | | VGQKLY | |
| PF-545 | A. niger, 50 μM | RESKLIAMADMIRRRI | 227 |
| | B. subtilis, 25 μM | | |
| | MRSA, 50 μM | | |
| PF-547 | Trubrum, 25 μM | WSRVPGHSDTGWKVWHRW | 228 |
| | B. subtilis, 25 μM | | |
| | S. mutans, 12.5 μM | | |
| PF-583 | MRSA, 50 μM | KFQGEFTNIGQSYIVSASHMSTSL | 229 |
| | S. epidermidis, 50 | NTGK | |
| | μM | | |
| PF-600 | E. coli, 50 μM | TKKIELKRFVDAFVKKSYENYILE | 230 |
| | S. pneumoniae, 50 | RELKKLIKAINEELPTK | |
| | μM | | |
| PF-606 | E. coli, 50 μM | FESKILNASKELDKEKKVNTALSF | 231 |
| | MRSA, 50 μM | NSHQDFAKAYQNGKI | |
| | S. epidermidis, 50 | | |
| | μM | | |
| | S. mutans, 50 μM | | |
| | S. pneumoniae, 50 | | |
| | μM | | |
| PF-672 | C. albicans, 1.56 | MRFGSLALVAYDSAIKHSWPRPSS | 232 |
| | μM | VRRLRM | |
| | Trubrum, 0.78 μM | | |
| | A. niger, 3 μM | | |
| | B. subtilis, 0.78 μM | | |
| | E. faecalis, 3.13 | | |
| | μM | | |
| | MRSA, 1.56 μM | | |
| | S. epidermidis, 0.39 | | |
| | μM | | |
| PGG | | GLLRRLRKKIGEIFKKYG | 233 |
| Protegrin-1 | | RGGRLCYCRRRFCVCVGR* | 234 |
| RIP | | YSPWTNF* | 235 |

A number of antimicrobial peptides are also disclosed in U.S. Pat. Nos. 7,271,239, 7,223,840, 7,176,276, 6,809,181, 6,699,689, 6,420,116, 6,358,921, 6,316,594, 6,235,973, 6,183,992, 6,143,498, 6,042,848, 6,040,291, 5,936,063, 5,830,993, 5,428,016, 5,424,396, 5,032,574, 4,623,733, which are incorporated herein by reference for the disclosure of particular antimicrobial peptides.

In certain embodiments, the antimicrobial peptide is a novispirin, a novispirin fragment or analog e.g., as shown above in Table 3. In certain embodiments constructs are contemplated where one or more of the targeting peptides described herein are attached (e.g., directly or through a linker) to a novispirin peptide, and/or to a truncated novispirin peptide G10, and/or to a modulated version of novispirin G10 designated G2 (KNLRIIRKGIHIIKKY (SEQ ID NO:169). In this case, the C terminal amino acids are removed and an internal arginine is eliminated to facilitate chemical synthesis. Novispirin G10 (the “parent molecule”) is an antimicrobial alpha-helical octadecapeptide structurally related to cathelicidins and other innate immunity peptides.

In certain embodiments the antimicrobial peptide consists of or comprises a novispirin peptide sequence as described in US Patent Pub. No: 2005/00245452 having the formula:

KNLRRX¹X²RKX³X⁴HIIKKYG

where X¹, X², X³, and X⁴ are independently selected from the group consisting of glycine, threonine, serine, glutamic acid, aspartic acid, isoleucine, D-alanine, and D-isoleucine, provided that not more than three of the X residues are isoleucine. Illustrative novispirin peptides according to this formula are shown in Table 4.

TABLE 4
Illustrative, but non-limiting
novispirin peptides.

| Sequence | SEQ ID NO |
|---|---|
| KNLRRGIRKIIHIIKK | 236 |
| KNLRRTIRKIIHIIKK | 237 |
| KNLRRSIRKIIHIIKK | 238 |
| KNLRREIRKIIHIIKK | 239 |
| KNLRRDIRKIIHIIKK | 240 |
| KNLRRAIRKIIHIIKK | 241 |
| KNLRRIIRKIIHIIKK | 242 |
| KNLRRIGRKIIHIIKK | 243 |
| KNLRRITRKIIHIIKK | 244 |
| KNLRRISRKIIHIIKK | 245 |
| KNLRRIERKIIHIIKK | 246 |
| KNLRRIDRKIIHIIKK | 247 |
| KNLRRIARKIIHIIKK | 248 |
| KNLRRIIRKIIHIIKK | 249 |
| KNLRRIIRKGIHIIKK | 250 |
| KNLRRIIRKTIHIIKK | 251 |
| KNLRRIIRKIIHIIKK | 252 |
| KNLRRIIRKEIHIIKK | 253 |
| KNLRRIIRKDIHIIKK | 254 |
| KNLRRIIRKAIHIIKK | 255 |
| KNLRRIIRKIIHIIKK | 256 |
| KNLRRIIRKIGHIIKK | 257 |
| KNLRRIIRKITHIIKK | 258 |
| KNLRRIIRKISHIIKK | 259 |
| KNLRRIIRKIEHIIKK | 260 |

In certain embodiments one or more of the C. difficile targeting peptides described herein are attached directly or through a linker to one or more of the antimicrobial peptides described above. However, the foregoing antimicrobial peptides are illustrative, but non-limiting. Other suitable antimicrobial peptides will be recognized by one of skill in the art.

Antibiotics.

In certain embodiments chimeric moieties are provided comprising one or more a targeting peptides that bind C. difficile (e.g. as described in Table 2) attached directly or through a linker to a small molecule antibiotic and/or to a carrier (e.g., a lipid or liposome, a polymer, etc.) that carries or contains a small molecule antibiotic. In certain embodiments the antibiotic can be an antibiotic conventionally used, or suggested for use, in the treatment of C. difficile infections. Such antibiotics include, but are not limited to metronidazole, bacitracin, vancomycin, fidaxomicin, nitazoxanide, rifaximin, and the like. Additionally, in view of the specific targeting provided by the targeting peptides described herein, other antibiotics can be used as well. Various illustrative, but non-limiting, antibiotics are shown in Table 5.

TABLE 5
Illustrative antibiotics for use as or in the effectors joined to
C. difficile targeting peptides.

| Class | Generic Name | BRAND NAME |
|---|---|---|
| Aminoglycosides | | |
| | Amikacin | AMIKIN® |
| | Gentamicin | GARAMYCIN® |
| | Kanamycin | KANTREX® |
| | Neomycin | |
| | Netilmicin | NETROMYCIN® |
| | Streptomycin | |
| | Tobramycin | NEBCIN® |
| | Paromomycin | HUMATIN® |
| Carbacephem | | |
| | Loracarbef | LORABID® |
| Carbapenems | | |
| | Ertapenem | INVANZ® |
| | Doripenem | FINIBAX® |
| | Imipenem/Cilastatin | PRIMAXIN® |
| | Meropenem | MERREM® |
| Cephalosporins | | |
| (First generation) | | |
| | Cefadroxil | DURICEF® |
| | Cefazolin | ANCEF® |
| | Cefalotin or Cefalothin | KEFLIN® |
| | Cefalexin | KEFLEX® |
| Cephalosporins | | |
| (Second | | |
| generation) | | |
| | Cefaclor | CECLOR® |
| | Cefamandole | MANDOLE® |
| | Cefoxitin | MEFOXIN® |
| | Cefprozil | CEFZIL® |
| | Cefuroxime | CEFTIN, ZINNAT® |
| Cephalosporins | | |
| (Third | | |
| generation) | | |
| | Cefixime | SUPRAX® |
| | Cefdinir | OMNICEF® |
| | Cefditoren | SPECTRACEF® |
| | Cefoperazone | CEFOBID® |
| | Cefotaxime | CLAFORAN® |
| | Cefpodoxime | |
| | Ceftazidime | FORTAZ® |
| | Ceftibuten | CEDAX® |
| | Ceftizoxime | |
| | Ceftriaxone | ROCEPHIN® |
| Cephalosporins | | |
| (Fourth | | |
| generation) | | |
| | Cefepime | MAXIPIME® |
| Cephalosporins | | |
| (Fifth | | |
| generation) | | |
| | Ceftobiprole | |
| Glycopeptides | | |
| | Teicoplanin | |
| | Vancomycin | VANCOCIN® |
| Macrolides | | |
| Azithromycin | Zithromax | |
| Clarithromycin | Biaxin | |
| Dirithromycin | | |
| Erythromycin | Erythocin, Erythroped | |
| Roxithromycin | | |
| Troleandomycin | | |
| Telithromycin | Ketek | |
| Monobactams | | |
| | Aztreonam | |
| Penicillins | | |
| | Amoxicillin | NOVAMOX®, |
| | | AMOXIL® |
| | Ampicillin | |
| | Azlocillin | |
| | Carbenicillin | |
| | Cloxacillin | |
| | Dicloxacillin | |
| | Flucloxacillin | FLOXAPEN® |
| | Mezlocillin | |
| | Meticillin | |
| | Nafcillin | |
| | Oxacillin | |
| | Penicillin | |
| | Piperacillin | |
| | Ticarcillin | |
| Polypeptides | | |
| | Bacitracin | |
| | Colistin | |
| | Polymyxin B | |
| Quinolones | | |
| | Mafenide | |
| | Prontosil (archaic) | |
| | Sulfacetamide | |
| | Sulfamethizole | |
| | Sulfanilimide (archaic) | |
| | Sulfasalazine | |
| | Sulfisoxazole | |
| | Trimethoprim | BACTRIM® |
| | Trimethoprim- | |
| | Sulfamethoxazole | |
| | (Co-trimoxazole) | |
| | (TMP-SMX) | |
| Tetracyclines | | |
| | Demeclocycline | |
| | Doxycycline | VIBRAMYCIN® |
| | Minocycline | MINOCIN® |
| | Oxytetracycline | TERRACIN® |
| | Tetracycline | SUMYCIN® |
| Natural products | | |
| | Antimicrobial herbal | |
| | extracts | |
| | Essential oils | |
| | Farnesol | |
| | Licorice root extracts | |
| | Glycyrrhizol A | |
| | Glycyrrhizol B | |
| | 6,8-diisoprenyl- | |
| | 5,7,4′- | |
| | trihydroxyisoflavone | |
| Others | | |
| | Arsphenamine | SALVARSAN® |
| | Chloramphenicol | CHLOROMYCETIN® |
| | Clindamycin | CLEOCIN® |
| | Lincomycin | |
| | Ethambutol | |
| | Fosfomycin | |
| | Fusidic acid | FUCIDIN® |
| | Furazolidone | |
| | Isoniazid | |
| | Linezolid | ZYVOX® |
| | Tedizolid | |
| | Metronidazole | FLAGYL® |
| | Mupirocin | BACTROBAN® |
| | Nitrofurantoin | MACRODANTIN®, |
| | | MACROBID® |
| | Platensimycin | |
| | Pyrazinamide | |
| | Quinupristin/Dalfopristin | SYNCERCID® |
| | Rifampin or Rifampicin | |
| | Tinidazole | |
| | Artemisinin | |
| | Fidaxomicin | |
| Antifungals | | |
| | Amphotericin B | |
| | Anidulafungin | |
| | Caspofungin acetate | |
| | Clotrimazole | |
| | Fluconazole | |
| | Flucytosine | |
| | Griseofulvin | |
| | Itraconazole | |
| | Ketoconazole | |
| | Micafungin | |
| | Miconazole | |
| | Nystatin | |
| | Pentamidine | |
| | Posaconazole | |
| | Terbinafine | |
| | Voriconazole | |
| Antimycobiotics | | |
| | Aminosalicylic Acid | |
| | Capreomycin | |
| | Clofazimine | |
| | Cycloserine | |
| | Ethionamide | |
| | Rifabutin | |
| | Rifapentine | |
| Antivirals | | |
| | Abacavir | |
| | Acyclovir | |
| | Adefovir | |
| | Amantadine | |
| | Atazanavir | |
| | Cidofovir | |
| | Darunavir | |
| | Didanosine | |
| | Docosanol | |
| | Efavirenz | |
| | Emtricitabine | |
| | Enfuvirtide | |
| | Entecavir | |
| | Etravirine | |
| | Famciclovir | |
| | Fomivirsen | |
| | Fosamprenavir | |
| | Foscarnet | |
| | Ganciclovir | |
| | Idoxuridine | |
| | Indinavir | |
| | Interferon alpha | |
| | Lamivudine | |
| | Lopinavir/ritonavir | |
| | Maraviroc | |
| | Nelfinavir | |
| | Nevirapine | |
| | Oseltamivir | |
| | Penciclovir | |
| | Peramivir | |
| | Raltegravir | |
| | Ribavirin | |
| | Rimantadine | |
| | Ritonavir | |
| | Saquinavir | |
| | Stavudine | |
| | Telbivudine | |
| | Tenofovir | |
| | Tipranavir | |
| | Trifluridine | |
| | Valacyclovir | |
| | Valganciclovir | |
| | Zanamivir | |
| | Zidovudine | |
| Anti-parasitics | | |
| | Albendazole | |
| | Artesunate | |
| | Atovaquone | |
| | Bephenium | |
| | hydroxynaphthoate | |
| | Chloroquine | |
| | Dapsone | |
| | Diethyl-carbamazine | |
| | Diloxanide furoate | |
| | Eflornithine | |
| | Emetine HCl | |
| | Furazolidone | |
| | Ivermectin | |
| | Lindane | |
| | Mebendazole | |
| | Mefloquine | |
| | Melarsoprol | |
| | Miltefosine | |
| | Niclosamide | |
| | Nifurtimox | |
| | Nitazoxanide | |
| | Oxamniquine | |
| | Paromomycin | |
| | Permethrin | |
| | Piperazine | |
| | Praziquantel | |
| | Primaquine | |
| | Pyrantel pamoate | |
| | Pyrimethamine | |
| | Proguanil | |
| | Quinacrine HCl | |
| | Quinidine | |
| | Quinine | |
| | Sodium Stibogluconate | |
| | Spiramycin | |
| | Thiabendazole | |
| | Tinidazole | |

Porphyrins and Non-Porphyrin Photosensitizers.

In certain embodiments, the C. difficile targeting peptides described herein (e.g., peptides shown in Table 2) can be attached to porphyrins and other photosensitizers. A photosensitizer is a drug or other chemical that increases photosensitivity of the organism (e.g., bacterium, yeast, fungus, etc.). Photosensitizers can be useful in photodynamic antimicrobial chemotherapy (PACT). In various embodiments PACT utilizes photosensitizers and light (e.g., visible, ultraviolet, infrared, etc.) in order to give a phototoxic response in the target organism(s), often via oxidative damage.

Currently, the major use of PACT is in the disinfection of blood products, particularly for viral inactivation, although more clinically-based protocols are used, e.g. in the treatment of oral infection or topical infection. The technique has been shown to be effective in vitro against bacteria (including drug-resistant strains), yeasts, viruses, parasites, and the like.

Attaching a targeting peptide described herein to the photosensitizer provides a means of specifically or preferentially targeting the photosensitizer(s) to particular species or strains(s) of microorganism (e.g., C. difficile).

A wide range of photosensitizers, both natural and synthetic are known to those of skill in the art (see, e.g., Wainwright (1998) J. Antimicrob. Chemotherap. 42: 13-28). Photosensitizers are available with differing physicochemical make-up and light-absorption properties. In various embodiments photosensitizers are usually aromatic molecules that are efficient in the formation of long-lived triplet excited states. In terms of the energy absorbed by the aromatic-system, this again depends on the molecular structure involved. For example, furocoumarin photosensitizers (psoralens) absorb relatively high energy ultraviolet (UV) light (c. 300-350 nm), whereas macrocyclic, heteroaromatic molecules such as the phthalocyanines absorb lower energy, near-infrared light.

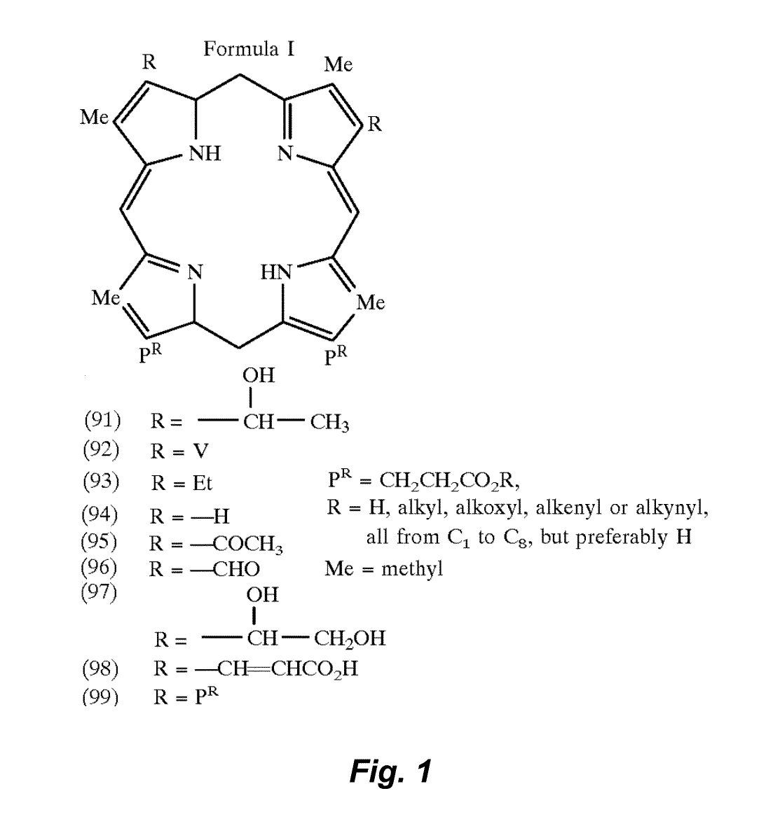

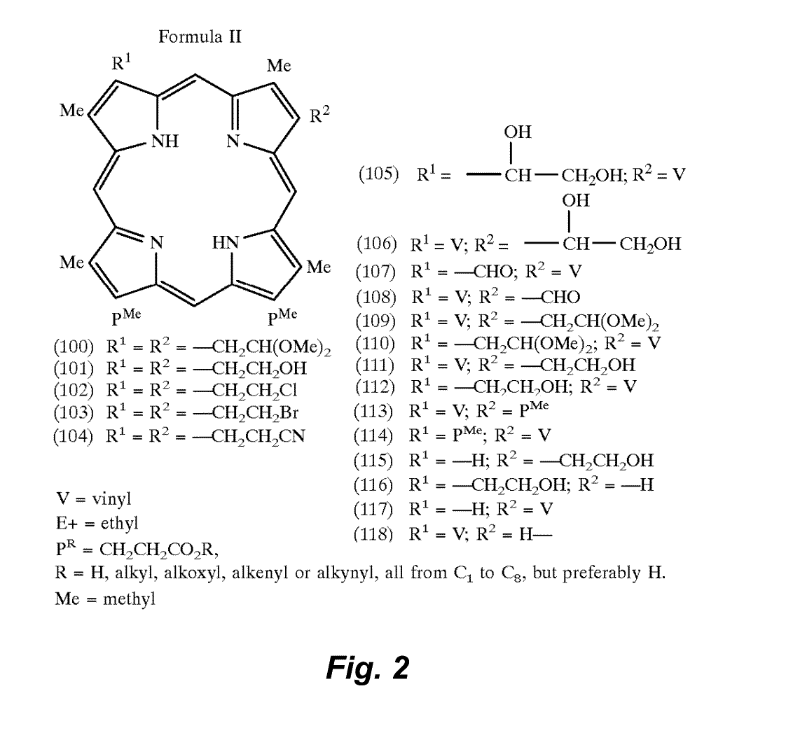

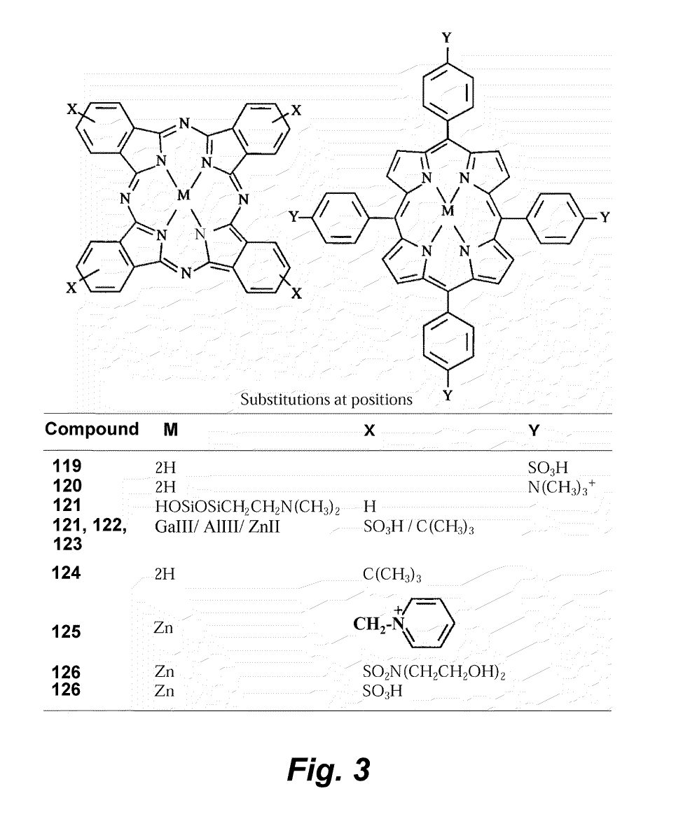

Illustrative photosensitizers include, but are not limited to porphyrinic macrocyles (especially porphyrins, chlorines, etc., see, e.g., FIGS. 1 and 2). In particular, metalloporphyrins, particularly a number of non-iron metalloporphyrins mimic haem in their molecular structure and are actively accumulated by bacteria via high affinity haem-uptake systems. The same uptake systems can be used to deliver antibiotic-porphyrin and antibacterial-porphyrin conjugates. Illustrative targeting porphyrins suitable for this purpose are described in U.S. Pat. No. 6,066,628 and shown herein in FIGS. 1 and 2.

An illustrative example of targeted porphyrins is shown in FIG. 13.

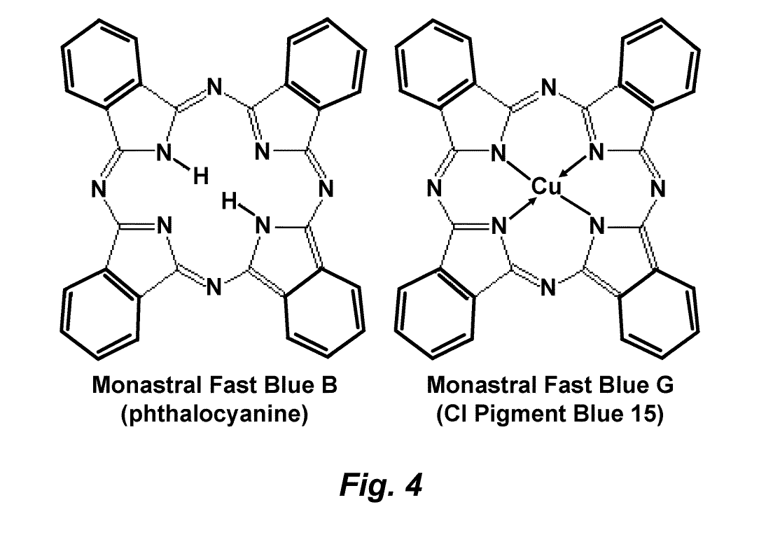

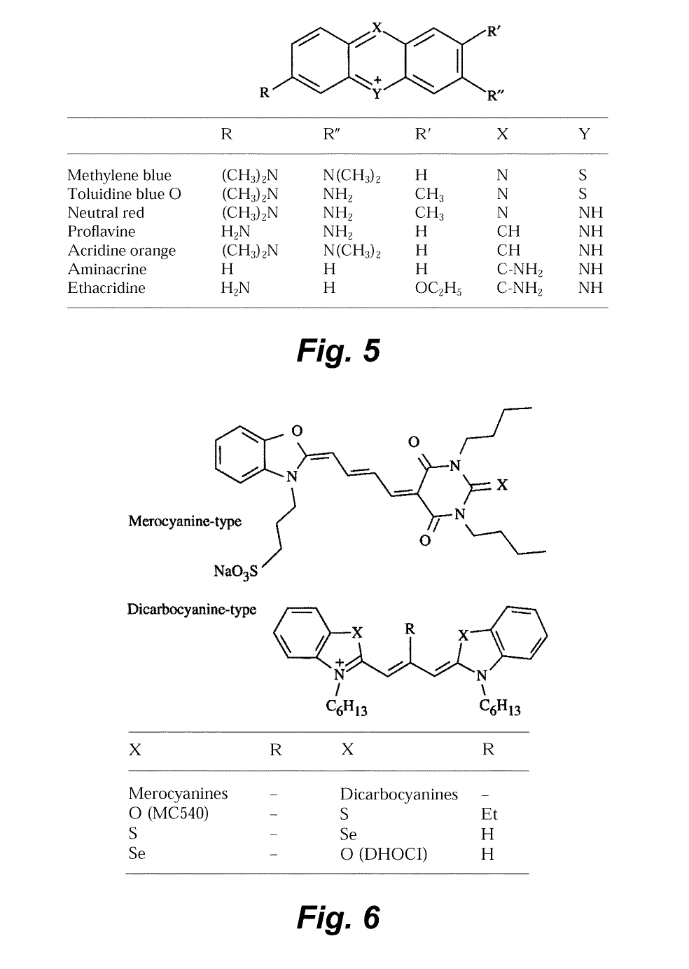

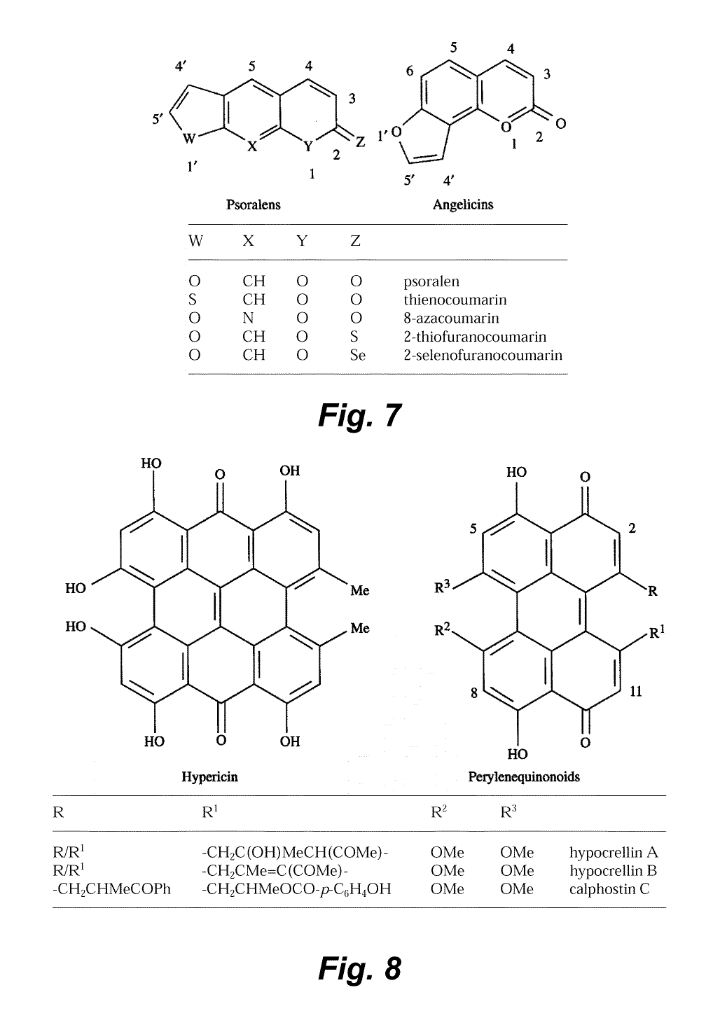

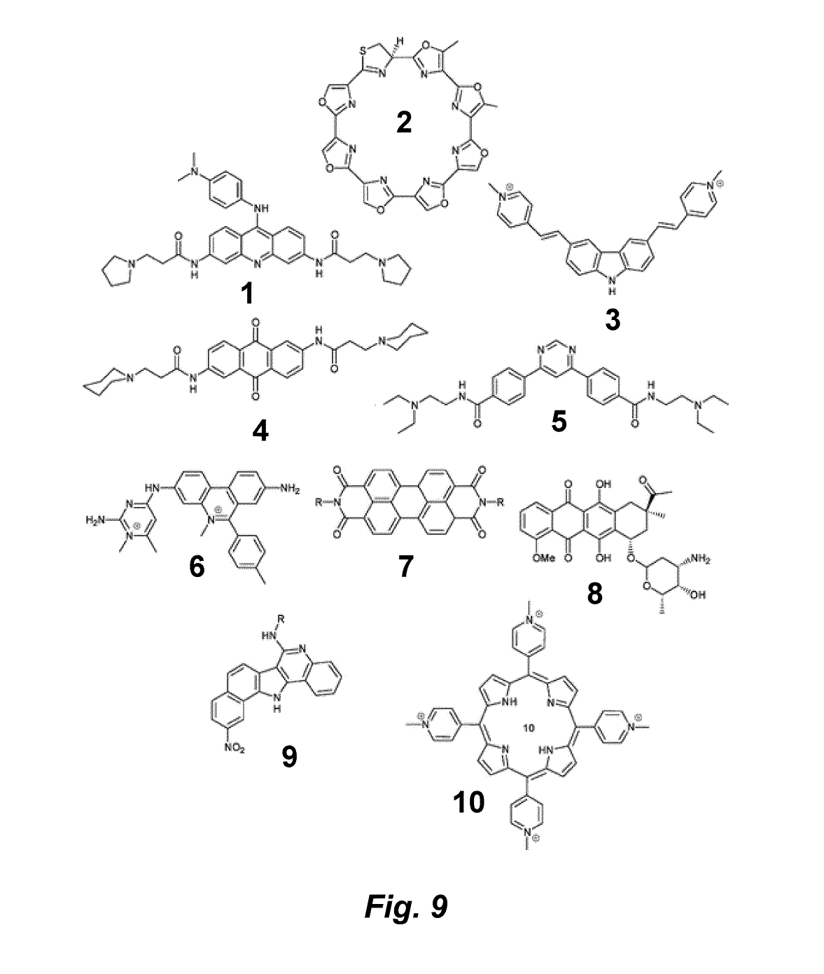

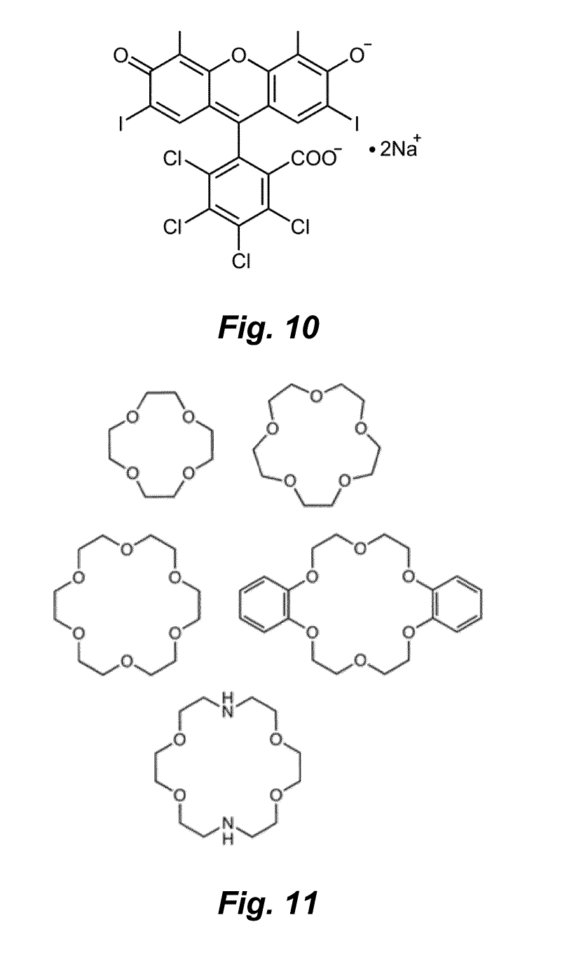

Other photosensitizers include, but are not limited to cyanines (see, e.g., FIG. 6) and phthalocyanines (see, e.g., FIG. 4), azines (see, e.g., FIG. 5) including especially methylene blue and toluidine blue, hypericin (see, e.g., FIG. 8), acridines (see, e.g., FIG. 9) including especially Rose Bengal (see, e.g., FIG. 10), crown ethers (see, e.g., FIG. 11), and the like. In certain embodiments, the photosensitizers include tin chlorin 6 and related compounds (e.g., other chlorines and tin porphyrins).

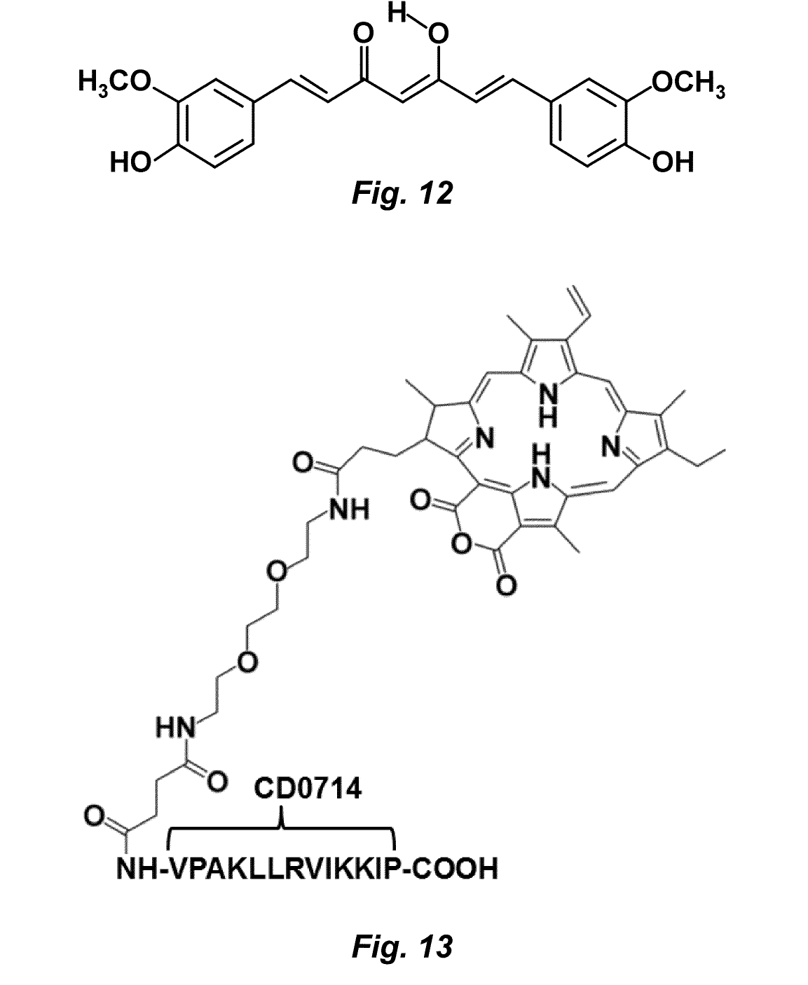

Another light-activated compound is cucumin (see, FIG. 12).

In certain embodiments the photosensitizers are toxic or growth inhibitors without light activation. For example, some non-iron metalloporphyrins (MPs) (see, e.g., FIGS. 1 and 2 herein) possess a powerful light-independent antimicrobial activity. In addition, haemin, the most well-known natural porphyrin, possesses a significant antibacterial activity that can be augmented by the presence of physiological concentrations of hydrogen peroxide or a reducing agent.

Typically, when activated by light, the toxicity or growth inhibition effect is substantially increased. Typically, they generate radical species that affect anything within proximity. In certain embodiments to get the best selectivity from targeted photosensitizers, anti-oxidants can be used to quench un-bound photosensitizers, limiting the damage only to cells where the conjugates have accumulated due to the targeting peptide. The membrane structures of the target cell act as the proton donors in this case.

In typical photodynamic antimicrobial chemotherapy (PACT) the targeted photosensitizer is “activated by the application of a light source (e.g., a visible light source, an ultraviolet light source, an infrared light source, etc.). PACT applications however need not be limited to topical use. Regions of the mouth, throat, nose, sinuses are readily illuminated. Similarly regions of the gut can readily be illuminated using endoscopic techniques. Other internal regions can be illumined using laparoscopic methods or during other surgical procedures. For example, in certain embodiments involving the insertion or repair or replacement of an implantable device (e.g., a prosthetic device) it contemplated that the device can be coated or otherwise contacted with a chimeric moiety comprising a targeting peptide attached to a photosensitizer as described herein. During the surgical procedure and/or just before closing, the device can be illuminated with an appropriate light source to activate the photosensitizer.

The targeted photosensitizers and uses thereof described herein are illustrative and not to be limiting. Using the teachings provided herein, other targeted photosensitizers and uses thereof will be available to one of skill in the art.

Ligands.

In certain embodiments the effector can comprise one or more ligands, epitope tags, and/or antibodies. In certain embodiments preferred ligands and antibodies include those that bind to surface markers on immune cells. Chimeric moieties utilizing such antibodies as effector molecules act as bifunctional linkers establishing an association between the immune cells bearing binding partner for the ligand or antibody and the target microorganism(s).

The terms “epitope tag” or “affinity tag” are used interchangeably herein, and refer to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, His₆ bound by Ni-NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO:261) epitope, c-myc antibodies (available from Sigma, St. Louis), the HNK-1 carbohydrate epitope, the HA epitope, the HSV epitope, the His₄ (SEQ ID NO:262), His₅ (SEQ ID NO:263), and His₆ (SEQ ID NO:264) epitopes that are recognized by the His epitope specific antibodies (see, e.g., QIAGEN GmbH), and the like. In addition, vectors for epitope tagging proteins are commercially available. Thus, for example, the pCMV-Tag1 vector is an epitope tagging vector designed for gene expression in mammalian cells. A target gene inserted into the pCMV-Tag1 vector can be tagged with the FLAG® epitope (N-terminal, C-terminal or internal tagging), the c-myc epitope (C-terminal) or both the FLAG (N-terminal) and c-myc (C-terminal) epitopes.

Lipids and Liposomes.

In certain embodiments the targeting peptides described herein (e.g., the peptides shown in Table 2) are attached to one or more microparticles or nanoparticles that can be loaded with an effector agent (e.g., a pharmaceutical, a label, etc.). In certain embodiments the microparticles or nanoparticles are lipidic particles. Lipidic particles are microparticles or nanoparticles that include at least one lipid component forming a condensed lipid phase. Typically, a lipidic nanoparticle has preponderance of lipids in its composition. Various condensed lipid phases include solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (see, e.g., The Structure of Biological Membranes, ed. by P. Yeagle, CRC Press, Bora Raton, F L, 1991). Lipidic microparticles include, but are not limited to a liposome, a lipid-nucleic acid complex, a lipid-drug complex, a lipid-label complex, a solid lipid particle, a microemulsion droplet, and the like. Methods of making and using these types of lipidic microparticles and nanoparticles, as well as attachment of affinity moieties, e.g., antibodies, to them are known in the art (see, e.g., U.S. Pat. Nos. 5,077,057; 5,100,591; 5,616,334; 6,406,713; 5,576,016; 6,248,363; Bondi et al. (2003) Drug Delivery 10: 245-250; Pedersen et al., (2006) Eur. J. Pharm. Biopharm. 62: 155-162, 2006 (solid lipid particles); U.S. Pat. Nos. 5,534,502; 6,720,001; Shiokawa et al. (2005) Clin. Cancer Res. 11: 2018-2025 (microemulsions); U.S. Pat. No. 6,071,533 (lipid-nucleic acid complexes), and the like).

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. Methods for liposome preparation are exhaustively described in several review articles such as Szoka and Papahadjopoulos (1980) Ann. Rev. Biophys. Bioeng., 9: 467, Deamer and Uster (1983) Pp. 27-51 In: Liposomes, ed. M. J. Ostro, Marcel Dekker, New York, and the like.

In various embodiments the liposomes include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. In certain embodiments, vesicle-forming lipids with diacyl chains, such as phospholipids, are preferred. One illustrative phospholipid is phosphatidylethanolamine (PE), which contains a reactive amino group convenient for coupling to the activated polymers. One illustrative PE is distearoyl PE (DSPE). Another example is non-phospholipid double chain amphiphilic lipids, such as diacyl- or dialkylglycerols, derivatized with a hydrophilic polymer chain.

In certain embodiments a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

Preparation of lipid-polymer conjugates containing these polymers attached to a suitable lipid, such as PE, have been described, for example in U.S. Pat. No. 5,395,

The liposomes can, optionally be prepared for attachment to one or more targeting peptides described herein. Here the lipid component included in the liposomes would include either a lipid derivatized with the targeting peptide, or a lipid having a polar-head chemical group, e.g., on a linker, that can be derivatized with the targeting peptide in preformed liposomes, according to known methods.

Methods of functionalizing lipids and liposomes with affinity moieties such as antibodies are well known to those of skill in the art (see, e.g., DE 3,218,121; Epstein et al. (1985) Proc. Natl. Acad. Sci., USA, 82:3688 (1985); Hwang et al. (1980) Proc. Natl. Acad. Sci., USA, 77: 4030; EP 52,322; EP 36,676; EP 88,046; EP 143,949; EP 142,641; Japanese patent application 83-118008; U.S. Pat. Nos. 4,485,045 and 4,544,545; and EP 102,324, all of which are incorporated herein by reference).

Agents that Physically Disrupt the Extracellular Matrix within a Community of Microorganisms

In certain embodiments the targeting peptides described herein (e.g., the peptides shown in Table 2) can be coupled to agents that physically disrupt the extracellular matrix within a community of microorganisms, for example a biofilm. In certain preferred embodiments, such an agent could be a bacterial cell-wall degrading enzyme, for example SAL-2, or Dispersin B, or any species of glycosidase, alginase, peptidase, proteinase, lipase, or DNA or RNA degrading enzyme or compound, for example rhRNase. Disruption of extracellular matrix of biofilms can result in clearance and therapeutic benefit.

The peptides can also be attached to antimicrobial proteins, such as Protein Inhibitor C or Colicin, or fragments thereof, for example the IIa domain of Colicin, or the heparin-binding domain of Protein Inhibitor C.

Polymeric Microparticles and/or Nanoparticles.

In certain embodiments the targeting peptides described herein (e.g., the peptides shown in Table 2) are attached to polymeric microparticles and/or nanoparticles and/or micelles.

Microparticle and nanoparticle-based drug delivery systems have considerable potential for treatment of various microorganisms. Technological advantages of polymeric microparticles or nanoparticles used as drug carriers are high stability, high carrier capacity, feasibility of incorporation of both hydrophilic and hydrophobic substances, and feasibility of variable routes of administration, including oral application and inhalation. Polymeric nanoparticles can also be designed to allow controlled (sustained) drug release from the matrix. These properties of nanoparticles enable improvement of drug bioavailability and reduction of the dosing frequency.

Polymeric nanoparticles are typically micron or submicron (<1 μm) colloidal particles. This definition includes monolithic nanoparticles (nanospheres) in which the drug is adsorbed, dissolved, or dispersed throughout the matrix and nanocapsules in which the drug is confined to an aqueous or oily core surrounded by a shell-like wall. Alternatively, in certain embodiments, the drug can be covalently attached to the surface or into the matrix.

Polymeric microparticles and nanoparticles are typically made from biocompatible and biodegradable materials such as polymers, either natural (e.g., gelatin, albumin) or synthetic (e.g., polylactides, polyalkylcyanoacrylates), or solid lipids. In the body, the drug loaded in nanoparticles is usually released from the matrix by diffusion, swelling, erosion, or degradation. One commonly used material is poly(lactide-co-glycolide) (PLG).

Methods of fabricating and loading polymeric nanoparticles or microparticles are well known to those of skill in the art. Thus, for example, Matsumoto et al. (1999) Intl. J. Pharmaceutics, 185: 93-101 teaches the fabrication of poly(L-lactide)-poly(ethylene glycol)-poly(L-lactide) nanoparticles, Chawla et al. (2002) Intl. J. Pharmaceutics 249: 127-138, teaches the fabrication and use of poly(e-caprolactone) nanoparticles delivery of tamifoxen, and Bodmeier et al. (1988) Intl. J. Pharmaceutics, 43: 179-186, teaches the preparation of poly(D,L-lactide) microspheres using a solvent evaporation method.” Intl. J. Pharmaceutics, 1988, 43, 179-186. Other nanoparticle formulations are described, for example, by Williams et al. (2003) J. Controlled Release, 91: 167-172; Leroux et al. (1996) J. Controlled Release, 39: 339-350; Soppimath et al. (2001) J. Controlled Release, 70: 1-20; Brannon-Peppas (1995) Intl. J. Pharmaceutics, 116: 1-9; and the like.

Detectable Labels.

In certain embodiments chimeric moieties are provided comprising one or more targeting peptides (e.g., as described in Table 2) attached directly or through a linker to a detectable label. Such chimeric moieties are effective for detecting the presence and/or quantity, and/or location of the microorganism(s) (e.g., S. mutans) to which the targeting peptide is directed. Similarly these chimeric moieties are useful to identify cells and/or tissues and/or food stuffs and/or other compositions that are infected with the targeted microorganism(s).

Detectable labels suitable for use in such chimeric moieties include any composition detectable by spectroscopic, photochemical, biochemical, immunochemical, electrical, optical, or chemical means. Illustrative useful labels include, but are not limited to, biotin for staining with labeled streptavidin conjugates, avidin or streptavidin for labeling with biotin conjugates fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like. Patents teaching the use of such labels include, for example, U.S. Pat. Nos. 3,817,837; 3,850,752; 3,939,350; 3,996,345; 4,277,437; 4,275,149; and 4,366,241.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

In various embodiments spin labels are provided by reporter molecules with an unpaired electron spin which can be detected by electron spin resonance (ESR) spectroscopy. Illustrative spin labels include organic free radicals, transitional metal complexes, particularly vanadium, copper, iron, and manganese, and the like. Exemplary spin labels include, for example, nitroxide free radicals.

Means of detecting such labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label.

Targeting Enhancers/Opsonins

In certain embodiments compositions are contemplated that incorporate a targeting enhancer (e.g., an opsonin) along with one or more targeting peptides. Targeting enhancers include moieties that increase binding affinity, and/or binding specificity, and/or internalization of a moiety by the target cell/microorganism.

Accordingly, in certain embodiments, a targeting peptide and/or a targeted antimicrobial molecule (e.g., a STAMP) comprise a targeting peptide described herein attached (e.g., conjugated) to an opsonin. When bound to a target cell through the targeting peptide, the opsonin component encourages phagocytosis and destruction by resident macrophages, dendritic cells, monocytes, or PMNs. Opsonins contemplated for conjugation can be of a direct or indirect type.

Direct opsonins include, for example, any bacterial surface antigen, PAMP (pathogen-associated molecular pattern), or other molecule recognized by host PRRs (pathogen recognizing receptors). Opsonins can include, but are not limited to, bacterial protein, lipid, nucleic acid, carbohydrate and/or oligosaccharide moieties.

In certain embodiments opsonins include, but are not limited to, N-acetyl-D-glucosamine (GlcNAc), N-acetyl-D-galactosamine (GlaNAc), N-acetylglucosamine-containing muramyl peptides, NAG-muramyl peptides, NAG-NAM, peptidoglycan, teichoic acid, lipoteichoic acid, LPS, o-antigen, mannose, fucose, ManNAc, galactose, maltose, glucose, glucosamine, sucrose, mannosamine, galactose-alpha-1,3-galactosyl-beta-1,4-N-acetyl glucosamine, or alpha-1,3-gal-gal, or other sugars.

In certain embodiments, opsonins include indirect opsonins. Indirect opsonins function through binding to a direct opsonin already present. For example an Fc portion of an antibody, a sugar-binding lectin protein (example MBL), or host complement factors (example C3b, C4b, iC3b).

In certain embodiments the opsonin is galactose-alpha-1,3-galactosyl-beta-1,4-N-acetyl glucosamine, or alpha-1,3-gal-gal.

Other examples of opsonin molecules include, but are not limited to antibodies (e.g., IgG and IgA), components of the complement system (e.g., C3b, C4b, and iC3b), mannose-binding lectin (MBL) (initiates the formation of C3b), and the like.

Methods of coupling an opsonin to a targeting peptide are well known to those of skill in the art (see, e.g., discussion below regarding attachment of effectors to targeting moieties).

### Peptide Preparation.

The peptides described herein can be chemically synthesized using standard chemical peptide synthesis techniques or, particularly where the peptide does not comprise “D” amino acid residues, the peptide can be recombinantly expressed. Where the “D” polypeptides are recombinantly expressed, a host organism (e.g., bacteria, plant, fungal cells, etc.) can be cultured in an environment where one or more of the amino acids is provided to the organism exclusively in a D form. Recombinantly expressed peptides in such a system then incorporate those D amino acids.

In certain embodiments, D amino acids can be incorporated in recombinantly expressed peptides using modified amino acyl-tRNA synthetases that recognize D-amino acids.

In certain embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In one illustrative, but non-limiting, embodiment the peptides can be synthesized by the solid phase peptide synthesis procedure using a benzhyderylamine resin (Beckman Bioproducts, 0.59 mmol of NH₂/g of resin) as the solid support. The COOH terminal amino acid (e.g., t-butylcarbonyl-Phe) is attached to the solid support through a 4-(oxymethyl)phenacetyl group. This is a more stable linkage than the conventional benzyl ester linkage, yet the finished peptide can still be cleaved by hydrogenation. Transfer hydrogenation using formic acid as the hydrogen donor can be used for this purpose.

It is noted that in the chemical synthesis of peptides, particularly peptides comprising D amino acids, the synthesis usually produces a number of truncated peptides in addition to the desired full-length product. Thus, the peptides are typically purified using standard methods well known to those of skill in the art, e.g., HPLC.

D-amino acids, beta amino acids, non-natural amino acids, and the like can be incorporated at one or more positions in the peptide simply by using the appropriately derivatized amino acid residue in the chemical synthesis. Modified residues for solid phase peptide synthesis are commercially available from a number of suppliers (see, e.g., Advanced Chem Tech, Louisville; Nova Biochem, San Diego; Sigma, St Louis; Bachem California Inc., Torrance, etc.). The D-form and/or otherwise modified amino acids can be completely omitted or incorporated at any position in the peptide as desired. Thus, for example, in certain embodiments, the peptide can comprise a single modified acid, while in other embodiments, the peptide comprises at least two, generally at least three, more generally at least four, most generally at least five, preferably at least six, more preferably at least seven or even all modified amino acids. In certain embodiments, essentially every amino acid is a D-form amino acid.

As indicated above, the peptides and/or fusion proteins described herein can also be recombinantly expressed. Accordingly, in certain embodiments, the antimicrobial peptides and/or targeting moieties, and/or fusion proteins of this invention are synthesized using recombinant expression systems. Generally this involves creating a DNA sequence that encodes the desired peptide or fusion protein, placing the DNA in an expression cassette under the control of a particular promoter, expressing the peptide or fusion protein in a host, isolating the expressed peptide or fusion protein and, if required, renaturing the peptide or fusion protein.

DNA encoding the peptide(s) or fusion protein(s) described herein can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding the peptides or fusion proteins described herein can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will typically be operably linked to appropriate expression control sequences for each host. For E. coli this can include a promoter such as the T7, trp, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant peptide(s) or fusion protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification, Academic Press, Inc. N.Y.). Substantially pure compositions of at least about 90 to 95% homogeneity are preferred, and 98 to 99% or more homogeneity are most preferred.

One of skill in the art would recognize that after chemical synthesis, biological expression, or purification, the peptide(s) or fusion protein(s) may possess a conformation substantially different than desired native conformation. In this case, it may be necessary to denature and reduce the peptide or fusion protein and then to cause the molecule to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the peptide(s) and/or fusion protein(s) proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Joining C. difficile Targeting Peptides to Other Moieties.

Chemical Conjugation.

Chimeric moieties are formed by joining one or more of the targeting peptides described herein to one or more effectors. In certain embodiments the targeting peptides are attached directly to the effector(s) via naturally occurring reactive groups or the targeting peptide(s) and/or the effector(s) can be functionalized to provide such reactive groups.

In various embodiments the targeting peptides are attached to effector(s) via one or more linking agents. Thus, in various embodiments the targeting peptides and the effector(s) can be conjugated via a single linking agent or multiple linking agents. For example, the targeting peptide and the effector can be conjugated via a single multifunctional (e.g., bi-, tri-, or tetra-) linking agent or a pair of complementary linking agents. In another embodiment, the targeting peptide and the effector are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions useful in forming chimeric moieties include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C.

In certain embodiments, the linking agent comprises a chelator. For example, the chelator comprising the molecule, DOTA (DOTA=1,4,7,10-tetrakis(carboxymethyl)-1,4,7,10-tetraazacyclododecane), can readily be labeled with a radiolabel, such as Gd³⁺ and ⁶⁴Cu, resulting in Gd³⁺-DOTA and ⁶⁴Cu-DOTA respectively, attached to the targeting peptide. Other suitable chelates are known to those of skill in the art, for example, 1,4,7-triazacyclononane-N,N′,N″-triacetic acid (NOTA) derivatives being among the most well-known (see, e.g., Lee et al. (1997) Nucl Med Biol. 24: 2225-23019).

A “linker” or “linking agent” as used herein, is a molecule that is used to join two or more molecules. In certain embodiments the linker is typically capable of forming covalent bonds to both molecule(s) (e.g., the targeting peptide and the effector). Suitable linkers are well known to those of skill in the art and include, but are not limited to, straight or branched-chain carbon linkers, heterocyclic carbon linkers, or peptide linkers. In certain embodiments the linkers can be joined to the constituent amino acids through their side groups (e.g., through a disulfide linkage to cysteine). However, in certain embodiments, the linkers will be joined to the alpha carbon amino and carboxyl groups of the terminal amino acids.

A bifunctional linker having one functional group reactive with a group on one molecule (e.g., a targeting peptide), and another group reactive on the other molecule (e.g., an antimicrobial peptide), can be used to form the desired conjugate. Alternatively, derivatization can be performed to provide functional groups. Thus, for example, procedures for the generation of free sulfhydryl groups on peptides are also known (See U.S. Pat. No. 4,659,839).

In certain embodiments the linking agent is a heterobifunctional crosslinker comprising two or more different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In one embodiment, the heterobifunctional crosslinker is SMCC.

Many procedures and linker molecules for attachment of various molecules to peptides or proteins are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

Fusion Proteins.

In certain embodiments where the targeting peptide and the moiety to be attached (e.g., an antimicrobial peptide (AMP)) are both peptides or both comprise peptides, the chimeric moiety can be chemically synthesized or expressed as a recombinant fusion protein.

In certain embodiments the fusion proteins (e.g., anti-C. difficile STAMPs) are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

In certain embodiments, DNA encoding fusion proteins of the present invention may be cloned using DNA amplification methods such as polymerase chain reaction (PCR). Thus, for example, the nucleic acid encoding a targeting antibody, a targeting peptide, and the like is PCR amplified, using a sense primer containing the restriction site for NdeI and an antisense primer containing the restriction site for HindIII. This produces a nucleic acid encoding the targeting sequence and having terminal restriction sites. Similarly an effector and/or effector/linker/spacer can be provided having complementary restriction sites. Ligation of sequences and insertion into a vector produces a vector encoding the fusion protein.

While the targeting peptides and other moieties (e.g., AMPs) can be directly joined together, one of skill will appreciate that they can be separated by a peptide spacer/linker consisting of one or more amino acids. Generally the spacer will have no specific biological activity other than to join the proteins or to preserve some minimum distance or other spatial relationship between them. However, the constituent amino acids of the spacer may be selected to influence some property of the molecule such as the folding, net charge, or hydrophobicity.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification, Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the fusion protein may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

As indicated above, in various embodiments an amino acid or a peptide linker/spacer is used to join the one or more targeting peptides to one or more effector(s). In various embodiments the peptide linker is relatively short, typically less than about 10 amino acids, preferably less than about 8 amino acids and more preferably about 2 or about 3 to about 5, or to about 6, or to about 7, or to about 8, or to about 9, or to about 10 amino acids. In certain embodiments the C. difficile targeting peptide is attached directly to an effector (e.g., an AMP). In certain embodiments the linker is a single amino acid (e.g., A, R, N, D, C, E, Q, G, H, I, L, K, M, F, P, S, T, W, Y, or V). in certain embodiments the linker is 2 amino acids, or 3 amino acids, or 4 amino acids, or 5 amino acids, or 6 amino acids, or 7 amino acids, or 8 amino acids, or 9 amino acids, or 10 amino acids, or 11 amino acids, or 12 amino acids, or 13 amino acids, or 14 amino acids, or 15 amino acids, or 16 amino acids, or 17 amino acids, or 18 amino acids, or 19 amino acids, or 20 amino acids, or 21 amino acids, or 22 amino acids, or 23 amino acids, or 24 amino acids, or 250 amino acids in length. Suitable illustrative linkers include, but are not limited to the linkers shown in Table 6.

TABLE 6
Illustrative, but non-limiting, peptide and non-peptide
linkers.

| Linker | SEQ ID NO: |
|---|---|
| P | |
| G | |
| GG | |
| GS | |
| AAA | |
| SGG | |
| SAT | |
| PYP | |
| ASA | |
| GAG | |
| GGG | |
| GGGG | 265 |
| GGGGG | 266 |
| GGGGGG | 267 |
| GGGGGGG | 268 |
| GGGGGGGG | 269 |
| GGAG | 270 |
| GGGAG | 271 |
| GSGS | 272 |
| GSGSGS | 273 |
| GSGSGSGS | 274 |
| GSGSGSGSGS | 275 |
| GSGSGSGSGSGS | 276 |
| PSPSP | 277 |
| KKKK | 278 |
| RRRR | 279 |
| ASASA | 280 |
| PSGSP | 281 |
| GGSGGS | 282 |
| GGGGS | 283 |
| GGGGS GGGGS | 284 |
| GGGGS GGGGS GGGGS | 285 |
| GGGGS GGGGS GGGGS GGGGS | 286 |
| GGGGS GGGGS GGGGS GGGGS GGGGS | 287 |
| GGGGS GGGGS GGGGS GGGGS GGGGS GGGGS | 288 |
| 2-nitrobenzene or O-nitrobenzyl | |
| Nitropyridyl disulfide | |
| Dioleoylphosphatidylethanolamine (DOPE) | |
| S-acetylmercaptosuccinic acid | |
| 1, 4, 7, 10-tetraazacyclododecane-1, 4, 7, | |
| 10-tetracetic acid (DOTA) | |
| β-glucuronide and β-glucuronide variants | |
| Poly(alkylacrylic acid) | |
| Benzene-based linkers (for example: 2,5-Bis | |
| (heloxy)-1,4-bis[2,5-bis(hexyloxy)-4-formyl- | |
| phenylenevinylene]benzene) and like molecules | |
| Disulfide linkages | |
| Poly(amidoamine) or like dendrimers linking | |
| multiple target and killing peptides in one | |
| molecule | |
| Carbon nanotubes | |
| Hydrazone and hydrazone variant linkers | |
| PEG of any chain length | |
| Succinate, formate, acetate, butyrate, other | |
| like organic acids | |
| Aldols, alcohols, or enols | |
| Peroxides | |
| alkane or alkene groups of any chain length | |
| One or more porphyrin or dye molecules containing | |
| free amino and carboxylic acid groups | |
| One or more DNA or RNA nucleotides, including | |
| polyamine and polycarboxyl-containing variants | |
| Inulin, sucrose, glucose, or other single, di or | |
| polysaccharides | |
| Linoleic acid or other polyunsaturated fatty | |
| acids | |
| Variants of any of the above linkers containing | |
| halogen or thiol groups | |

(All amino-acid-based linkers could be L, D, combinations of L and D forms, β-form, and the like)

Multiple Targeting Peptides and/or Effectors.

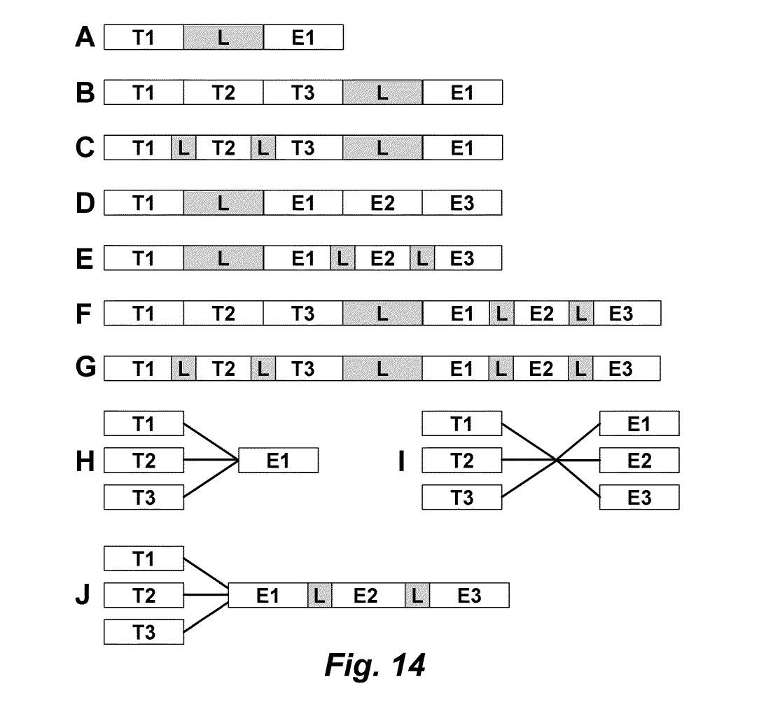

As indicated above, in certain embodiments, the constructs described herein can comprise multiple targeting peptides attached to a single effector or multiple effectors attached to a single targeting peptide, or multiple targeting peptides attached to multiple effectors.

Where the construct is a fusion protein this is easily accomplished by providing multiple domains that are targeting domains attached to one or more effector domains. FIG. 14 schematically illustrates a few, but not all, configurations. In various embodiments the multiple targeting domains and/or multiple effector domains can be attached to each other directly or can be separated by linkers (e.g., amino acid or peptide linkers as described above).

When the chimeric construct is a chemical conjugate linear or branched configurations (e.g., as illustrated in FIG. 14) are readily produced by using branched linkers (e.g., dendritic polymers also known as dendrimers), and/or multifunctional linkers and/or a plurality of different linkers.

Dendritic polymers include, but are not limited to, symmetrical and unsymmetrical branching dendrimers, cascade molecules, arborols, and the like. PAMAM dendrimers (see, e.g. U.S. Patent Publication No: 2012/0219496) are symmetric, in that the branch arms are of equal length. The branching occurs at the hydrogen atoms of a terminal —NH₂ group on a preceding generation branch.

Even though not formed by regular sequential addition of branched layers, hyperbranched polymers, e.g., hyper branched polyols, may be equivalent to a dendritic polymer where the branching pattern exhibits a degree of regularity approaching that of a dendrimer.

Topological polymers, with size and shape controlled domains, are dendrimers that are associated with each other (as an example covalently bridged or through other association) through their reactive terminal groups, which are referred to as “bridged dendrimers.” When more than two dense dendrimers are associated together, they are referred to as “aggregates” or “dense star aggregates.” Therefore, dendritic polymers include bridged dendrimers and dendrimer aggregates. Dendritic polymers encompass both generationally monodisperse and generationally polydisperse solutions of dendrimers. The dendrimers in a monodisperse solution are substantially all of the same generation, and hence of uniform size and shape. The dendrimers in a polydisperse solution comprise a distribution of different generation dendrimers.

Dendritic polymers also encompass surface modified dendrimers. For example, the surface of a PAMAM dendrimer may be modified by the addition of an amino acid (e.g., lysine or arginine). As used herein, the term “generation” when referring to a dendrimer means the number of layers of repeating units that are added to the initiator core of the dendrimer. For example, a 1st generation dendrimer comprises an initiator core and one layer of the repeating unit, and a 2nd generation dendrimer comprises an initiator core and two layers of the repeating unit, etc. Sequential building of generations (i.e., generation number and the size and nature of the repeating units) determines the dimensions of the dendrimers and the nature of their interior.

Methods for linking dendrimers to biological substrates (e.g., peptides) are well known to those of skill in the art, and include the use of a cross-linking agent. For example, thiol-reactive species can be made by coupling the dendrimer hydroxyl group to the isocyanate end of the bi-functional cross-linker, N-(p-maleimidophenyl)isocyanate, leaving a thiol-reactive maleimide for coupling to peptides. Examples of a cross-linking agent include, but are not limited to, a homobifunctional cross linker, a heterobifunctional cross-linker, a linear polymer, a branched polymer, a nanoparticle, a nucleic acid, a an amino acid, a peptide, or a combination thereof. In a particular embodiment, the cross-linking agent is a homobifunctional amine-reactive cross-linking agent, for example, NHS-PEG-NHS. The presence of PEG spacer arm may help maintain the water solubility of formed dendrimer clusters.

In some embodiments, the cross-linking reaction may be performed by a click-chemistry, preferably, a thiolene chemistry. Other suitable click chemistries, known to one of skilled in the art, for example, but not limited to, Staudinger ligation and Cu-catalyzed terminal alkyne-azide cycloaddition, may also be used.

Protecting Groups.

While the various peptides described herein may be shown with no protecting groups, in certain embodiments they can bear one, two, three, four, or more protecting groups. In various embodiments, the protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus.

Without being bound by a particular theory, it was discovered that addition of a protecting group, particularly to the carboxyl and in certain embodiments the amino terminus can improve the stability and efficacy of the peptide.

A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. In certain particularly preferred embodiments, the protecting groups include, but are not limited to alkyl chains as in fatty acids, propionyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g., groups having the formula: CH₃—(CH₂)ₙ—CO— where n ranges from about 1 to about 20, preferably from about 1 to about 16 or 18, more preferably from about 3 to about 13, and most preferably from about 3 to about 10.

In certain embodiments, the protecting groups include, but are not limited to alkyl chains as in fatty acids, propionyl, formyl, and others. Particularly preferred carboxyl protecting groups include amides, esters, and ether-forming protecting groups. In one embodiment, an acetyl group is used to protect the amino terminus and/or an amino group is used to protect the carboxyl terminus (i.e., amidated carboxyl terminus). In certain embodiments blocking groups include alkyl groups of various lengths, e.g., groups having the formula: CH₃—(CH₂)ₙ—CO— where n ranges from about 3 to about 20, preferably from about 3 to about 16, more preferably from about 3 to about 13, and most preferably from about 3 to about 10.

In certain embodiments, the acid group on the C-terminal can be blocked with an alcohol, aldehyde or ketone group and/or the N-terminal residue can have the natural amide group, or be blocked with an acyl, carboxylic acid, alcohol, aldehyde, or ketone group.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, xanthyl (Xan), trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), cyclohexyloxy (cHxO), t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). In illustrative embodiment, for example, acetylation is accomplished during the synthesis when the peptide is on the resin using acetic anhydride. Amide protection can be achieved by the selection of a proper resin for the synthesis. For example, a rink amide resin can be used. After the completion of the synthesis, the semipermanent protecting groups on acidic bifunctional amino acids such as Asp and Glu and basic amino acid Lys, hydroxyl of Tyr are all simultaneously removed. The peptides released from such a resin using acidic treatment comes out with the n-terminal protected as acetyl and the carboxyl protected as NH₂ and with the simultaneous removal of all of the other protecting groups.

Where amino acid sequences are disclosed herein, amino acid sequences comprising, one or more protecting groups, e.g., as described above (or any other commercially available protecting groups for amino acids used, e.g., in boc or fmoc peptide synthesis) are also contemplated.

### Incorporation of Chemoattractant Peptide Sequence.

In various embodiments any of the targeting peptides described herein is attached to a chemoattractant peptide and/or any of the STAMPs described herein further comprises a chemoattractant peptide sequence/domain.

In certain embodiments the chemoattractant peptide (chemoattractant domain) comprises or consists of an amino acid sequence characterized by the motif XKYX(P/V)M (SEQ ID NO:289) where X is any amino acid and M is methionine or D-methionine. In certain embodiments X is any naturally occurring amino acid or the D form of any naturally occurring amino acid. In certain embodiments the chemoattractant peptide (or domain) comprises or consists of a leukocyte chemoattractant peptide sequence with specificity for FPR1 and/or FPR2. In certain embodiments the amino acid sequence of the chemoattractant peptide or domain comprises or consists of the amino acid sequence WKYMVM (SEQ ID NO:290) (aka W-peptide). In certain embodiments this sequence consists of all “L” amino acids (is an L-peptide sequence). In certain embodiments this sequence consists of all “D” amino acids (is a D-peptide sequence). In certain embodiments this sequence is selective for FPR2 and FPR3. In certain embodiments In certain embodiments the amino acid sequence of the chemoattractant peptide or domain comprises or consists of the amino acid sequence WKYMV(dM) (SEQ ID NO:291) where dM is D-methionine and the other resides are all L residues.

In certain embodiments any of these chemoattractant domains are attached to the amino or carboxyl terminus of a targeting peptide described herein. The attachment can be chemical conjugation or the peptide can be expressed or synthesized as a fusion protein with or without an amino acid linker between the targeting peptide and the chemoattractant sequence.

In certain embodiments any of these chemoattractant domains are attached to the amino or carboxyl terminus of, or inserted into, a STAMP (e.g., a STAMP comprising a targeting domain described herein). The attachment can be chemical conjugation or the peptide can be expressed or synthesized as a fusion protein with or without an amino acid linker between the STAMP domain(s) and the chemoattractant sequence.

### Peptide Circularization.

In certain embodiments the peptides described herein are circularized/cyclized to produce cyclic peptides. Cyclic peptides, as contemplated herein, include head/tail, head/side chain, tail/side chain, and side chain/side chain cyclized peptides. In addition, peptides contemplated herein include homodet, containing only peptide bonds, and heterodet containing in addition disulfide, ester, thioester-bonds, or other bonds.

The cyclic peptides can be prepared using virtually any art-known technique for the preparation of cyclic peptides. For example, the peptides can be prepared in linear or non-cyclized form using conventional solution or solid phase peptide syntheses and cyclized using standard chemistries. Preferably, the chemistry used to cyclize the peptide will be sufficiently mild so as to avoid substantially degrading the peptide. Suitable procedures for synthesizing the peptides described herein as well as suitable chemistries for cyclizing the peptides are well known in the art.

In various embodiments cyclization can be achieved via direct coupling of the N- and C-terminus to form a peptide (or other) bond, but can also occur via the amino acid side chains. Furthermore it can be based on the use of other functional groups, including but not limited to amino, hydroxy, sulfhydryl, halogen, sulfonyl, carboxy, and thiocarboxy. These groups can be located at the amino acid side chains or be attached to their N- or C-terminus.

Accordingly, it is to be understood that the chemical linkage used to covalently cyclize the peptides of the invention need not be an amide linkage. In many instances it may be desirable to modify the N- and C-termini of the linear or non-cyclized peptide so as to provide, for example, reactive groups that may be cyclized under mild reaction conditions. Such linkages include, by way of example and not limitation amide, ester, thioester, CH₂—NH, etc. Techniques and reagents for synthesizing peptides having modified termini and chemistries suitable for cyclizing such modified peptides are well-known in the art.

Alternatively, in instances where the ends of the peptide are conformationally or otherwise constrained so as to make cyclization difficult, it may be desirable to attach linkers to the N- and/or C-termini to facilitate peptide cyclization. Of course, it will be appreciated that such linkers will bear reactive groups capable of forming covalent bonds with the termini of the peptide. Suitable linkers and chemistries are well-known in the art and include those previously described.

Cyclic peptides and depsipeptides (heterodetic peptides that include ester (depside) bonds as part of their backbone) have been well characterized and show a wide spectrum of biological activity. The reduction in conformational freedom brought about by cyclization often results in higher receptor-binding affinities. Frequently in these cyclic compounds, extra conformational restrictions are also built in, such as the use of D- and N-alkylated-amino acids, α,β-dehydro amino acids or α,α-disubstituted amino acid residues.

Methods of forming disulfide linkages in peptides are well known to those of skill in the art (see, e.g., Eichler and Houghten (1997) Protein Pept. Lett. 4: 157-164).

Reference may also be made to Marlowe (1993) Biorg. Med. Chem. Lett. 3: 437-44 who describes peptide cyclization on TFA resin using trimethylsilyl (TMSE) ester as an orthogonal protecting group; Pallin and Tam (1995) J. Chem. Soc. Chem. Comm. 2021-2022) who describe the cyclization of unprotected peptides in aqueous solution by oxime formation; Algin et al. (1994) Tetrahedron Lett. 35: 9633-9636 who disclose solid-phase synthesis of head-to-tail cyclic peptides via lysine side-chain anchoring; Kates et al. (1993) Tetrahedron Lett. 34: 1549-1552 who describe the production of head-to-tail cyclic peptides by three-dimensional solid phase strategy; Tumelty et al. (1994) J. Chem. Soc. Chem. Comm. 1067-1068, who describe the synthesis of cyclic peptides from an immobilized activated intermediate, where activation of the immobilized peptide is carried out with N-protecting group intact and subsequent removal leading to cyclization; McMurray et al. (1994) Peptide Res. 7: 195-206) who disclose head-to-tail cyclization of peptides attached to insoluble supports by means of the side chains of aspartic and glutamic acid; Hruby et al. (1994) Reactive Polymers 22: 231-241) who teach an alternate method for cyclizing peptides via solid supports; and Schmidt and Langer (1997) J. Peptide Res. 49: 67-73, who disclose a method for synthesizing cyclotetrapeptides and cyclopentapeptides.

These methods of peptide cyclization are illustrative and non-limiting. Using the teaching provide herein, other cyclization methods will be available to one of skill in the art.

### Identification/Verification of Active Peptides

The active AMPS, STAMPs and the like can be identified and/or validated using an in vitro screening assay. Additionally, despite certain apparent limitations of in vitro susceptibility tests, clinical data indicate that a good correlation exists between minimal inhibitory concentration (MIC) test results and in vivo efficacy of antibiotic compounds (see, e.g., Murray et al. (1994) Antimicrobial Susceptibility Testing, Poupard et al., eds., Plenum Press, New York; Knudsen et al. (1995) Antimicrob. Agents Chemother. 39(6): 1253-1258; and the like). Thus, AMPS useful for treating infections and diseases related thereto are also conveniently identified by demonstrated in vitro antimicrobial activity against specified microbial targets, e.g., C. difficile).

Typically, the in vitro antimicrobial activity of antimicrobial agents is tested using standard NCCLS bacterial inhibition assays, or MIC tests (see, National Committee on Clinical Laboratory Standards “Performance Standards for Antimicrobial Susceptibility Testing,” NCCLS Document M100-S5 Vol. 14, No. 16, December 1994; “Methods for dilution antimicrobial susceptibility test for bacteria that grow aerobically-Third Edition,” Approved Standard M7-A3, National Committee for Clinical Standards, Villanova, Pa.).

It will be appreciated that other assays as are well known in the art or that will become apparent to those having skill in the art upon review of this disclosure may also be used to identify active STAMPs. Such assays include, for example, the assay described in Lehrer et al. (1988) J. Immunol. Meth., 108: 153 and Steinberg and Lehrer, “Designer Assays for Antimicrobial Peptides: Disputing the ‘One Size Fits All’ Theory,” In: Antibacterial Peptide Protocols, Shafer, Ed., Humana Press, N.J. Generally, active peptides of the invention will exhibit MICs (as measured using the assays described in the examples) of less than about 100 μM, preferably less than about 80 or 60 μM, more preferably about 50 μM or less, about 25 μM or less, or about 15 μM or less, or about 10 μM or less.

### Administration and Formulations.

Pharmaceutical Formulations.

In certain embodiments, the constructs described herein (e.g., C. difficile-targeting peptides attached to antimicrobial peptide(s), targeting peptides attached to detectable label(s), etc.) are administered to a mammal in need thereof, to a cell, to a tissue, to a composition (e.g., a stool sample), etc.). In various embodiments the compositions can be administered to detect and/or locate, and/or quantify the presence of particular microorganisms, microorganism populations, biofilms comprising particular microorganisms, and the like. In various embodiments the compositions can be administered to inhibit particular microorganisms, microorganism populations, biofilms comprising particular microorganisms, and the like.

These active agents (e.g., STAMPs) described herein can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids.

Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein are useful for the detection and/or treatment of C. difficile infections. In certain embodiments administration is parenteral, oral, nasal (or otherwise inhaled), rectal, or local administration, for detection and/or quantification, and or localization, and/or prophylactic and/or therapeutic treatment of infection (e.g., microbial infection). The compositions can be administered in a variety of unit dosage forms depending upon the method of administration. Suitable unit dosage forms, include, but are not limited to powders, tablets, pills, capsules, lozenges, pulmonary dosage forms (e.g., pulmonary dosage forms such as solutions for nebulizers, micronized powders for metered-dose inhalers, and the like), suppositories, patches, nasal sprays, injectables, implantable sustained-release formulations, lipid complexes, etc.

The active agents (e.g., STAMPs) described herein can also be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, BHT (butylated hydroxytoluene), chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, hydroxypropylmethylcellulose acetate succinate, and Eudragit (Evonik, Germany; methacrylic-acrylic copolymers).

Other physiologically acceptable compounds include wetting agents, emulsifying agents, dispersing agents or preservatives that are particularly useful for preventing the growth or action of microorganisms. Various preservatives are well known and include, for example, phenol and sorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain therapeutic applications, the compositions of this invention are administered, e.g., orally or rectally administered to a patient suffering from infection or at risk for infection or prophylactically to prevent C. difficile infections and associated pathologies in an amount sufficient to prevent and/or cure and/or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms in) the patient.

The dosage of active agent(s) can vary widely, and will be selected primarily based on activity of the active ingredient(s), body weight and the like in accordance with the particular mode of administration selected and the patient's needs. In various embodiments dosages can be provided ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or prophylactic regimen in a particular subject or group of subjects.

In certain embodiments, the active agents of this invention are administered to the oral cavity. This is readily accomplished by the use of lozenges, aersol sprays, mouthwash, coated swabs, and the like.

In certain embodiments the active agents of this invention are administered systemically (e.g., orally, or as an injectable) in accordance with standard methods well known to those of skill in the art. In other preferred embodiments, the agents, can also be delivered through the skin using conventional transdermal drug delivery systems, or transdermal drug delivery systems utilizing minimally invasive approaches (e.g., in combination with devices enabling microporation of upper layers of skin). Illustrative transdermal delivery systems include, but are not limited to transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While the invention is described with respect to use in humans, it is also suitable for animal, e.g., veterinary use. Thus certain preferred organisms include, but are not limited to humans, non-human primates, canines, equines, felines, porcines, ungulates, largomorphs, and the like.

Nanoemulsion Formulations.

In certain embodiments chimeric moieties (e.g., STAMPs) as described herein are formulated in a nanoemulsion. Nanoemulsions include, but are not limited to oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to:

Surfactant micelles—micelles composed of small molecules surfactants or detergents (e.g., SDS/PBS/2-propanol) which are suitable for predominantly hydrophobic peptides.

Polymer micelles—micelles composed of polymer, copolymer, or block copolymer surfactants (e.g., Pluronic L64/PBS/2-propanol) which are suitable for predominantly hydrophobic peptides;

Blended micelles: micelles in which there is more than one surfactant component or in which one of the liquid phases (generally an alcohol or fatty acid compound) participates in the formation of the micelle (e.g., Octanoic acid/PBS/EtOH) which are suitable for predominantly hydrophobic peptides;

Integral peptide micelles—blended micelles in which the peptide serves as an auxiliary surfactant, forming an integral part of the micelle (e.g., amphipathic peptide/PBS/mineral oil) which are suitable for amphipathic peptides; and

Pickering (solid phase) emulsions—emulsions in which the peptides are associated with the exterior of a solid nanoparticle (e.g., polystyrene nanoparticles/PBS/no oil phase) which are suitable for amphipathic peptides.

Illustrative water in oil (W/O) nanoemulsions include, but are not limited to:

Surfactant micelles—micelles composed of small molecules surfactants or detergents (e.g., dioctyl sulfosuccinate/PBS/2-propanol, Isopropylmyristate/PBS/2-propanol, etc.) which are suitable for predominantly hydrophilic peptides;

Polymer micelles—micelles composed of polymer, copolymer, or block copolymer surfactants (e.g., PLURONIC® L121/PBS/2-propanol), which are suitable for predominantly hydrophilic peptides;

Blended micelles—micelles in which there is more than one surfactant component or in which one of the liquid phases (generally an alcohol or fatty acid compound) participates in the formation of the micelle (e.g., capric/caprylic diglyceride/PBS/EtOH) which are suitable for predominantly hydrophilic peptides;

Integral peptide micelles—blended micelles in which the peptide serves as an auxiliary surfactant, forming an integral part of the micelle (e.g., amphipathic peptide/PBS/polypropylene glycol) which are suitable for amphipathic peptides; and

Pickering (solid phase) emulsions—emulsions in which the peptides are associated with the exterior of a solid nanoparticle (e.g., chitosan nanoparticles/no aqueous phase/mineral oil) which are suitable for amphipathic peptides.

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al., (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution, or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (i.e., constitutes 30-90% of the total volume of the final emulsion), more preferably 50-80%.

In certain embodiments the alcohol, when present, is ethanol.

While the present invention is not limited by the nature of the surfactant, in some preferred embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some preferred embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, an d5,152,923 and in Fanun et al. (2009) Microemulsions: Properties and Applications (Surfactant Science), CRC Press, Boca Ratan Fla.

Formulations Optimizing Activity.

In certain embodiments, formulations are selected to optimize binding specificity, and/or binding avidity, and/or antimicrobial activity, and/or stability/conformation of the targeting peptide, antimicrobial peptide, chimeric moiety, and/or STAMP. In this regard, it was a surprising discovery that the activity of certain STAMPs, and presumably the constituent targeting peptides and/or antimicrobial peptides was optimized in the presence of a salt. Accordingly, certain embodiments are contemplated where the targeting peptide and/or antimicrobial peptide, and/or STAMP is formulated in combination with one or more salts. The formulations disclosed herein, however, are not limited to those containing salt(s). Embodiments, are also contemplated where the targeting peptide and/or antimicrobial peptide, and/or STAMP is formulated without the presence of a salt.

In certain embodiments, sodium chloride plus a little potassium chloride resulted in the best activity of the salts tested. However, other salts, e.g., CaCl₂, MgCl₂, MnCl₂ also enhanced activity. Accordingly, in certain embodiments, it is contemplated that the targeting peptide(s), and/or antimicrobial peptide(s), and/or chimeric moieties, and/or STAMPs are formulated with one or more salts.

In certain embodiments suitable salts include any of a number of pharmaceutically acceptable salts. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, besylate, glucoheptonate, lactobionate, and laurylsulphonate salts and the like (see, e.g., Berge et al. (1977) J. Pharm. Sci. 66: 1-19), although it is noted that citrate salts appear to inhibit the activity of certain STAMPs.

In certain embodiments pharmaceutically acceptable salts of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenyl acetic, glutamic, benzoic, salicyclic, sulfanilic, 2-acetoxybenzoic, fumaric, toluenesulfonic, methanesulfonic, benzenesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra; and Stahl and Wermuth (2002) Handbook of Pharmaceutical Salts: Properties, Selection, and Use, Wiley-VCH, Zurich, Switzerland).

In various embodiments, the salt is simply a sodium chloride and/or a potassium chloride and can readily be prepared, for example, as a phosphate buffered saline (PBS) solution. In certain embodiments, the salt concentration is comparable to that found in 0.5×PBS to about 2.5×PBS, more preferably from about 0.5×PBS to about 1.5×PBS. In certain embodiments optimum activity has been observed in 1×PBS.

In various embodiments, the pH of the formulation ranges from about pH 5.0 to about pH 8.5, preferably from about pH 6.0 to about pH 8.0, more preferably from about pH 7.0 to about pH 8.0. In certain embodiments the pH is about pH 7.4.

While optimum results have been observed for certain STAMPs using a PBS buffer system, other buffer systems are also acceptable. Such buffers include, but are not limited to sulfate buffers, carbonate buffers, Tris buffers, CHAPS buffers, PIPES buffers, and the like, as long as the salt is included.

In various embodiments, the targeting peptide, and/or antimicrobial peptide, and/or chimeric moiety, and/or STAMP is present in the formulation at a concentration ranging from about 1 nM, to about 1, 10, or 100 mM, more preferably from about 1 nM, about 10 nM, about 100 nM, about 1 μM, or about 10 μM to about 50 μM, about 100 μM, about 200 μM, about 300 μM, about 400 μM, or about 500 μM, preferably from about 1 μM, about 10 μM, about 25 μM, or about 50 μM to about 1 mM, about 10 mM, about 20 mM, or about 5 mM, most preferably from about 10 μM, about 20 μM, or about 50 μM to about 100 μM, about 150 μM, or about 200 μM.

### Targeting Delivery to the Colon

In various embodiments, the compositions described herein are formulated for specific or preferential delivery to the colon. Approaches for targeted/preferential delivery of drugs (including proteins and peptides) to the colon include, but are not limited to: 1) pH sensitive polymer coatings; 2) Delayed (time controlled) release systems, 3) Microbial triggered drug delivery; 4) Pressure controlled drug delivery stems; an 5) Colon targeted delivery systems; 6) Osmotic controlled drug delivery; and the like.

A) pH Sensitive Polymer Coatings.

In the stomach, pH ranges between 1 and 2 during fasting but increases after eating (Rubinstein (1995) Crit. Rev. Ther. Drug Carrier Syst., 12(2-3): 101-149). The pH is about 6.5 in the proximal small intestine, and about 7.5 in the distal small intestine. From the ileum to the colon, pH declines significantly. It is about 6.4 in the cecum. However, pH values as low as 5.7 have been measured in the ascending colon in healthy volunteers. The pH in the transverse colon is 6.6 and 7.0 in the descending colon. Use of pH dependent polymers is based on these differences in pH levels.

Polymers described as pH dependent in colon specific drug delivery have low solubility or are insoluble at low pH levels, but become increasingly more soluble as pH rises (see, e.g., Ashord et al. (1993) J Control Release, 26: 213-220). Illustrative pH sensitive coatings are described, for example in PCT Publication No: WO1995011032A1. Illustrative materials described therein include pH-sensitive polymers that do not dissolve in the lower pH environs of the stomach and the upper portions of the small intestine (pHs lower than about 6.5), but that disintegrate or dissolve at the pH commonly found in the latter portions of the small intestine or in the proximal region of the colon, e.g., above pH 6.5. Such polymers include polymethacrylates (e.g., EUDRAGIT® Type S, or combinations of EUDRAGIT® Types L and S and FS30D, (Evonik, Darmstadt, West Germany), hydroxypropyl methylcellulose phthalate, shellac, hydroxypropylmethylcellulose acetate succinate, and polyvinyl acetate phthalate. The pH at which such pH-sensitive polymers begin to dissolve and the thickness of coating determines the site in the intestinal lumen at which the encapsulated drug is released. Typically, higher pH dissolution points and increased amounts of pH-sensitive polymer will increase the distance the unit dosage form will travel in the small intestine and colon prior to release of the drug. For certain compositions of this invention, preferred pH-sensitive enteric materials dissolve only at a pH of greater than about 6.5, more preferred enteric materials dissolve only at pH of greater than about 6.8; also preferred are enteric materials which dissolve only at a pH of greater than about 7. An suitable pH-sensitive material is a polymethacrylate polymer (EUDRAGIT® S) with a pH dissolution value of about pH 7, and especially EUDRAGIT® FS30D designed for ileum and upper colon delivery.

Various pH-sensitive coatings that achieve delivery in the colon have been described in patents such as U.S. Pat. No. 4,910,021, WO 9001329, U.S. Pat. No. 6,068,859, U.S. Pat. Nos. 5,175,003, and 5,316,774 which are incorporated herein by reference for the pH sensitive coatings described herein.

U.S. Pat. No. 5,484,610 discloses terpolymers or monomes such as alkyl acrylates or methacrylates, 1,3-diene monomers, α-methyl styrene, halogenated olefins, vinyl esters, acrylonitrile, methacrylonitrile, N-vinyl carbazole and the like. The terpolymers are sensitive to pH and temperature and are useful carriers for conducting bioactive agents through the gastric juices of the stomach in a protected form. The terpolymers swell at the higher physiologic pH of the intestinal tract causing release of the bioactive agents into the intestine. The terpolymers are linear and are typically made up of 35 to 99 wt % of a temperature sensitive component, which imparts to the terpolymer LCST (lower critical solution temperature) properties below body temperatures, 1 to 30 wt % of a pH sensitive component having a pKₐ in the range of from 2 to 8 that functions through ionization or deionization of carboxylic acid groups to prevent the bioactive agent from being lost at low pH but allows bioactive agent release at physiological pH of about 7.4 and a hydrophobic component that stabilizes the LCST below body temperatures and compensates for bioactive agent effects on the terpolymers.

Terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, and the like, and a preparation method therefore. The pH-sensitive polymers are prepared by introduction of sulfonamide groups, various in pKa, to hydrophilic groups of polymers either through coupling to the hydrophilic groups of polymers, such as acrylamide, N,N-dimethylacrylamide, acrylic acid, N-isopropylacrylamide and the like or copolymerization with other polymerizable monomers. These pH-sensitive polymers may have a structure of linear polymer, grafted copolymer, hydrogel or interpenetrating network polymer.

U.S. Pat. No. 5,656,292 discloses a composition for pH dependent or pH regulated controlled release of active ingredients especially drugs. The composition consists of a compactable mixture of the active ingredient and starch molecules substituted with acetate and dicarboxylate residues. One preferred dicarboxylate acid is succinate. The average substitution degree of the acetate residue is at least 1 and 0.2-1.2 for the dicarboxylate residue. The starch molecules can have the acetate and dicarboxylate residues attached to the same starch molecule backbone or attached to separate starch molecule backbones.

U.S. Pat. Nos. 5,554,147, 5,788,687, and 6,306,422 disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used.

Mathiowitz et al. U.S. Pat. No. 6,365,187 discloses bioadhesive polymers in the form of, or as a coating on, microcapsules containing drugs or bioactive substances that can serve for therapeutic, or diagnostic purposes in diseases of the gastrointestinal tract. The polymeric microspheres typically have a bioadhesive force of at least 11 mN/cm² (110 N/m²) Techniques for the fabrication of bioadhesive microspheres, as well as a method for measuring bioadhesive forces between microspheres and selected segments of the gastrointestinal tract in vitro are also described. Methods described in U.S. Pat. No. 6,365,187 provide a means to establish a correlation between the chemical nature, the surface morphology and the dimensions of drug-loaded microspheres on one hand and bioadhesive forces on the other, allowing the identification of the most promising materials from a relatively large group of natural and synthetic polymers that should be used for making bioadhesive microspheres.

It has surprisingly been discovered that the disadvantageous swelling of materials such as amylose can be controlled by a pH dependent material. The pH dependent material such as an acrylic polymer (e.g., EUDRAGIT S®). The amylose can be provided in the form of a high amylose starch, for example Hylon 7 or Eurylon 7 (a starch having about 70% by weight amylose. It has been found that a mix of two polymers at an appropriate ratio, applied as a film coating on to a core, minimizes drug release in the stomach and small intestine. Subsequent drug release in the colon occurs by the combined active physiological triggers: e.g., by EUDRAGIT® S dissolution and amylose digestion. The proportion of the first material to the second material may in some circumstances be up to 50:50, preferably up to 65:35 and most preferably from 15:85 to 30:70.

B) Delayed/Time-Controlled Release Systems

Time controlled release system (TCRS) such as sustained or delayed release dosage forms are useful drug release systems for colonic deliver.

Enteric coated time-release press coated (ETP) tablets, can be composed of three components, a drug containing core tablet (rapid release function), the press coated swellable hydrophobic polymer layer (Hydroxy propyl cellulose layer (HPC), time release function) and an enteric coating layer (acid resistance function). Tyipcally, the tablet does not release the drug in the stomach due to the acid resistance of the outer enteric coating layer. After gastric emptying, the enteric coating layer rapidly dissolves and the intestinal fluid begins to slowly erode the press coated polymer (HPC) layer. When the erosion front reaches the core tablet, rapid drug release occurs since the erosion process takes a long time as there is no drug release period (lag phase) after gastric emptying. The duration of lag phase is controlled either by the weight or composition of the polymer (HPC) layer.

C) Microbially Triggered Drug Delivery to Colon

The microflora of the colon is in the range of 10¹¹-10¹² CFU/mL, consisting mainly of anaerobic bacteria, e.g., bacteroides, bifidobacteria, eubacteria, clostridia, enterococci, enterobacteria and ruminococcus etc. This microflora fulfills its energy needs by fermenting various types of substrates that have been left undigested in the small intestine, e.g. di- and tri-saccharides, polysaccharides etc. For this fermentation, the microflora produces a large number of enzymes like glucoronidase, xylosidase, arabinosidase, galactosidase, nitroreductase, azareductase, deaminase, and urea dehydroxylase. Because of the presence of the biodegradable enzymes only in the colon, the use of biodegradable polymers for colon-specific drug delivery provides a more site-specific approach as compared to many other approaches.

Typically, these polymers shield the drug from the environments of stomach and small intestine, and are able to deliver the drug to the colon. On reaching the colon, they undergo assimilation by micro-organism, or degradation by enzyme or break down of the polymer back bone leading to a subsequent reduction in their molecular weight and thereby loss of mechanical strength. They are then unable to hold the drug entity any longer.

i) Prodrug Approach for Drug Delivery to Colon

In certain embodiments drug delivery to the colon can be achieved by formulating the active agent(s) (e.g., STAMPs) as a prodrug. Typically, prodrugs are pharmacologically inactive (or reduced activity) derivatives of a parent drug molecule that exploits spontaneous or enzymatic transformation in vivo to release the active drug. For colonic delivery, the prodrug is typically designed to undergo minimal hydrolysis in the upper tracts of the GI tract and undergo enzymatic hydrolysis in the colon thereby releasing the active drug moiety from the drug carrier. Metabolism of azo compounds by intestinal bacteria is one of the most extensively studied bacterial metabolic processes. A number of other linkages susceptible to bacterial hydrolysis especially in the colon have been prepared where the drug is attached to hydrophobic moieties like amino acids, glucoronic acids, glucose, galactose, cellulose etc. Illustrative, but non-limiting prodrug forms include, but are not limited to azo conjugates, amino acid conjugates, saccharide carriers, glucose/galactose/cellobioside linkages, glucoruonide conjugates and the like.

In certain embodiments azo-polymers are used to form prodrugs for delivery to the colon. Both synthetic as well as naturally occurring polymers have been used for this purpose. Synthetic polymers have been used to form polymeric prodrug with azo linkage between the polymer and drug moiety. A number of these have been evaluated for CDDS. Various azo polymers have also been evaluated as coating materials over drug cores. These have been found to be similarly susceptible to cleavage by the azoreducatase in the large bowel. Coating of peptide capsules with polymers cross linked with azoaromatic group has been found to protect the drug from digestion in the stomach and small intestine. In the colon, the azo bonds are reduced, and the drug is released. Illustrative azopolymeric prodrugs include, but are not limited to copolymers of styrene with 2-hydroxyethylmethacrylate, hydrogels comprising 2-hydroxymethacrylate with 4 methacryloyloxy azobenzene, segmented polyurethane, aromatic azo bonds containing urethane analogs, and the like.

By way of illustration, PCT Patent Publication No: WO 1998001421 A1 describes hydrogel polymeric systems for the site specific delivery of peptide and protein drugs to the colon. The hydrogel protects the drug through the acid environment of the stomach, swells at a chemically controlled rate in the higher pH environment of the small intestine and is enzymatically degraded by azoreductases in the colon. To this end, a series of N,O-diacylhydroxylamine monomers were synthesized and incorporated into an aromatic azo crosslinked hydrophilic acrylamide/acrylic acid hydrogel network. Depending upon the structure, these N,O-diacylhydroxylamines function as either acid group protectants or cross-linking moieties that also protect an acid functionality at an acid pH but hydrolyze at higher pH ranges as found in the small intestine to expose free carboxylic acid groups.

iii) Polysaccharide Based Delivery Systems

Naturally occurring polysaccharides can also be used for targeting drugs to the colon since these polymers of monosaccharides are found in abundance, have wide availability, are inexpensive and are available in a variety of a structures with varied properties. They can be easily modified chemically, biochemically, and are highly stable, safe, nontoxic, hydrophilic and gel forming and in addition, are biodegradable.

Suitable polysaccharides include naturally occurring polysaccharides obtained from plant (e.g., guar gum, inulin), animal (e.g., chitosan, chondroitin sulfate), algal (e.g., alginates) or microbial (e.g., dextran) origin. The polysaccharides can be broken down by the colonic microflora to simple saccharides. Therefore, they fall into the category of “generally regarded as safe” (GRAS). Illustrative polysaccharide-based delivery systems include, but are not limited to chitosan (e.g., enteric coated chitosan capsules), chitosan derivatives (e.g., chitosan succinate, chitosan succinate used as a carrier matrix or capsule), pectin (e.g., used as a carrier matrix or capsule, amidated pectin/calcium pectinate (e.g., utilized as a matrix tablet with ethyl cellulose, or as a drug matrix additive), chondroitin sulfate and/or cross-linked chondroitin (e.g., as a matrix tablet), alginates (e.g., as a calcium salt on swellable beads), and the like.

D) Pressure Controlled Drug-Delivery Systems

As a result of peristalsis, higher pressures are encountered in the colon than in the small intestine. Pressure controlled delivery systems that exploit these high pressures have been developed (see, e.g., Takaya et al. (1998) J. Control. Release, 50(1-3): 111-122). Illustrative controlled colon-delivery capsules can be prepared using ethylcellulose, which is insoluble in water. In such systems, drug release occurs following the disintegration of a water-insoluble polymer capsule because of pressure in the lumen of the colon. The thickness of the ethylcellulose membrane, the capsule size and density control the rate of disintegration of the formulation.

E) Novel Colon Targeted Delivery Systems (CODES™)

CODES™ is a CDDS technology that was designed to avoid the inherent problems associated with pH or time dependent systems. CODES™ utilizes a combined approach of pH dependent and microbially triggered CDDS (see, e.g., U.S. Pat. No. 6,368,629). It utilizes a mechanism involving lactulose, which acts as a trigger for site specific drug release in the colon. The system typically comprises a traditional tablet core containing lactulose, that is overcoated with an acid soluble material (e.g., EUDRAGIT® E, and which is subsequently overcoated with an enteric material, (e.g., EUDRAGIT® L). The enteric coating protects the tablet while it is located in the stomach and then dissolves quickly following gastric emptying. The acid soluble material coating then protects the preparation as it passes through the alkaline pH of the small intestine. Once the tablet arrives in the colon, bacteria enzymatically degrade the oligosaccharide (lactulose) into organic acid. This lowers the pH surrounding the system sufficient to effect the dissolution of the acid soluble coating and subsequent drug release.

F) Osmotic Controlled Drug Delivery (ORDS-CT)

Osmotic Controlled Drug Delivery (ORDS-CT) (see, e.g., U.S. Pat. No. 4,904,474) can be used to target a drug locally to the colon for the treatment of disease or to achieve systemic absorption that is otherwise unattainable. The system can be a single osmotic unit or may incorporate as many as 5-6 push-pull units, each encapsulated within a hard gelatin capsule, Each bilayer push pull unit contains an osmotic push layer and a drug layer, both surrounded by a semipermeable membrane. An orifice is drilled through the membrane next to the drug layer. Immediately after the ORDS-CT is swallowed, the gelatin capsule containing the push-pull units dissolves. Because of its drug-impermeable enteric coating, each push-pull unit is prevented from absorbing water in the acidic aqueous environment of the stomach, and hence no drug is delivered. As the unit enters the small intestine, the coating dissolves in this higher pH environment (pH>7), water enters the unit causing the osmotic push compartment to swell, and concomitantly creates a flowable gel in the drug compartment. Swelling of the osmotic push compartment forces drug gel out of the orifice at a rate precisely controlled by the rate of water transport through the semipermeable membrane. By way of example, for treating ulcerative colitis, each push pull unit is designed with a 3-4 h post gastric delay to prevent drug delivery in the small intestine. Drug release begins when the unit reaches the colon. ORDS-CT units can maintain a constant release rate for up to 24 hours in the colon or can deliver drug over a period as short as four hours. Other phase transited systems also provide good tools for targeting drugs to the colon. Various in vitro/in vivo evaluation techniques have been developed and proposed to test the performance and stability of CDDS.

### Microorganism Detection.

As indicated above, the targeting peptides and/or STAMPs are useful in diagnostic compositions and methods to determine the presence or absence and/or to quantify the amount of C. difficile present in a biological sample, and the like.

For example, targeting peptide-antimicrobial peptide conjugates described herein (e.g. (STAMPs)) can be used as diagnostic reagents. STAMPs (and other targeted antimicrobial constructs described herein) have the ability to specifically bind to C. difficile, and permeabilize or disrupt the bacterial membrane such that cell impermeable dyes or other reagent (propidium iodide, etc.) may enter the microorganism or intracellular molecules or contents (ATP, DNA, Calcium, etc.) of the targeted microorganism are caused to be released into the environment for analysis. In one method a STAMP described herein can permeabilize or disrupt the membrane of a C. difficile, in a prepared culture or clinical sample. To the sample a cell impermeable dye (e.g., propidium iodide, etc.) is added to label and allow for detection of those microorganisms targeted by the STAMP. Cell permeable dyes (e.g. SYTO9) can also be added to label and detect the entire population of microorganisms in the sample. Labeled cells can then be quantified by fluorescence microscopy, fluorometry, flow cytometry or other method.

In another example, a STAMP treated sample is mixed with luciferase and luciferin that reacts with the ATP released from the STAMP treated cells and the resulting luminescence is used to detected and quantify targeted cells.

### Fecal Matter Transplants.

Fecal microbiota transplantation (FMT) also known as a fecal matter transplant or a stool transplant is the process of transplantation of fecal bacteria typically from a healthy individual into a recipient or an autologous transplant from an earlier stored sample into the same subject (Bakken et al. (2011) Clin. Gastroenterol. Hepatol., 9(12): 1044-1049). Various studies have shown fecal matter transplantion to provide effective treatment for patients suffering from Clostridium difficile infection (CDI), that can produce effects ranging from diarrhea to pseudomembranous colitis (Borody and Khoruts (2011) Nat. Rev. Gastroenterol. Hepatol. 9(2): 88-96). Additionally, beginning in 2000, various hypervirulent strains of C. difficile have emerged, that appear to be linked to the administration of antibiotics commonly used in empiric treatments (Gould and McDonald (2008) Crit. Care 12(1): 203). In the U.S. alone, an estimated 3 million new acute Clostridium difficile infections currently are diagnosed annually (Sailhamer et al. (2009) Arch. Surg. 144: 433-439). Of these, a subgroup will go on to develop fulminant CDI which results in approximately 300 deaths per day or almost 110,000 deaths per year (Jarvis et al. (2009) Am. J. Infect. Control, 37: 263-270). Due to the epidemic in North America and Europe, FMT has gained increasing prominence, with some experts calling for it to become first-line therapy for CDI (Brandt et al. (2011) J. Clin. Gastroenterol. 45: 655-657).

Typically fecal matter transplantation (fecal microbiota transplantation) involves restoration of the colonic microflora by introducing healthy bacterial flora through infusion of stool (e.g. by enema, colonoscopy, rectal suppository, oral capsule, etc.), obtained from a healthy human donor or from the same subject (e.g., before administration of antibiotics). Infusion of feces from healthy donors has been demonstrated in a randomized, controlled trial to be highly effective in treating recurrent C. difficile, and more effective than vancomycin alone (van Nood et al. (2013) N. Engl. J. Med., 368(5): 407-415).

Preparing for the procedure typically involves careful selection and screening of the donor and excluding those who test positive for certain diseases as well as any donor carrying any pathogenic gastrointestinal infectious agent. However it is believed that treatment of the fecal matter (stool) with one or more of the constructs described herein can reduce or eliminate viable C. difficile in the sample without substantially depleting or killing the other microbiota in the sample thereby improving safety and broadening the donor pool.

Accordingly in certain embodiments, a composition comprising fecal matter (e.g. stool or a composition derived from stool) for fecal transplantation is contemplated where the composition comprises fecal matter combined with a construct that kills C. difficile as described herein. Typically the construct will be present in an amount sufficient to reduce or eliminate viable C. difficile in the fecal matter. In various embodiments the fecal matter can comprise human stool or stool from a non-human mammal (e.g., a feline, a canine, a porcine, a bovine, an equine, a non-human primate, a largomorph, etc.).

In illustrative, but non-limiting embodiments, approximately 200-300 grams of fecal material treated with one or more of the anti-C. difficile constructs described herein is provided per treatment. Fresh stools have been recommended to be used within six hours, however frozen stool samples can also be used without loss of efficacy. There is evidence that the relapse rate is 2 fold greater when water is used as opposed to saline as the dilution agent. There is also some evidence that using infusions of greater than 500 ml produce a higher success rate compared to infusions using less than 200 ml of prepared material.

In various embodiments the procedure can involve single to multiple infusions (e.g. by enema, colonoscopy, rectal suppository, through a nasogastric or nasoduodenal tube, or via oral administration of encapsulated fecal matter) of the construct-treated fecal material preferably obtained from a healthy donor or the same subject, e.g., obtained before administration of certain antibiotics. Most fecal transplant patients with CDI recover clinically and their CDI can be eradicated after just one treatment (Kelly et al. (2012) J. Clin. Gastroenterol. 46(2): 145-149; Brandt et al. (2011) J. Clin. Gastroenterol. 45(8): 655-657). While C. difficile is easily eradicated with a single FMT infusion, however, this generally appears to not be the case with ulcerative colitis. Published experience of ulcerative colitis treatment with FMT largely shows that multiple and recurrent infusions are required to achieve prolonged remission or cure (Borody and Campbell (2011) Exp. Rev. Gastroenterol. Hepatol. 5(6): 653-655).

The fecal microbiota infusions can be administered via various routes depending on suitability and ease, although enema infusion is perhaps the simplest. Other routes include, but are not limited to colonoscopy, rectal suppository, through a nasogastric or nasoduodenal tube, or via oral administration of encapsulated fecal matter. There does not appear to be any significant methodological difference in efficacy between the various routes. Typically, repeat stool testing is performed on subjects to confirm eradication of CDI.

Medical treatment with antibiotics often is the cause of C. difficile in a subject. In certain embodiments an autologous fecal sample, can be provided from the subject before medical treatment, and this sample can be is stored under refrigeration. Should the patient subsequently develop a C. difficile infection, the sample is extracted with saline filtered and treated with the anti-C. difficile constructs described herein. The filtrate can be used directly or it can be freeze-dried and the resulting solid enclosed in enteric-coated capsules.

In certain embodiments, the subjects most likely to receive this treatment are those who have had at least three recurrences of C. difficile infection and have failed conventional therapies, including, for example a pulsed, tapered regimen of vancomycin. However, in certain embodiments, the treatment spectrum can be widened to include any subjects that are severely ill because of C. difficile infection, even if the current infection is their first episode. Some of these severely ill subjects could develop fulminant colitis, require colectomy, or even die. It is believed that such complications can be prevented by fecal transplantation earlier in these subjects.

Another group of subjects in whom fecal transplantation might be considered using the construct-treated stool samples described herein is any subject with C. difficile infection, regardless of the number of recurrences or the severity of the infection. In a presentation at the 2011 Annual Meeting of the American College of Gastroenterology (ACG), a group of researchers reported on 77 patients from 5 geographically disparate medical centers who had undergone fecal transplantation at least 3 months previously. These patients had suffered from C. difficile infection for a minimum of 3 months, with the average duration of symptoms being 11 months, and they had failed an average of 5 prior conventional treatments. When asked about their attitudes toward fecal transplantation as a treatment option, 97% said they would elect to undergo fecal transplantation again if they experienced another recurrence of C. difficile infection, and 53% of patients said they would prefer fecal transplantation as their first-line treatment, rather than antibiotic therapy.

In certain embodiments, the fecal transplantation using the construct-treated stool contemplated herein is performed in combination with antibiotic therapy (e.g. vancomycin treatment). In certain embodiments the antibiotic treatment will follow fecal transplantation. Without being bound to a particular theory, it is believed that subjects can respond to vancomycin after fecal transplantations because they now have sufficient diversity of bacteria to keep C. difficile in check once vancomycin lowered the C. difficile burden.

While fecal transplantation has been used primarily for treatment of C. difficile infection, it is believed to provide an effective treatment for other diseases. Clinicians have limited experience using fecal transplantation for a variety of gastroenterologic diseases including, but not limited to, ulcerative colitis, Crohn's disease, irritable bowel syndrome, and idiopathic constipation. It is contemplated that the construct-treated stool samples described herein find utility in the treatment of these conditions as well.

### Kits.

In another embodiment kits are provided for the inhibition of a C. difficile infection and/or for prevention of a C. difficile infection in a mammal. The kits typically comprise a container containing one or more of the active agents (i.e., the antimicrobial peptide(s) and/or chimeric construct(s)) described herein. In certain embodiments the active agent(s) can be provided in a unit dosage formulation (e.g., suppository, tablet, caplet, patch, etc.) and/or may be optionally combined with one or more pharmaceutically acceptable excipients.

In certain embodiments kits are provided for detecting and/or locating and/or quantifying certain target microorganisms (e.g., C. difficile) and/or cells or tissues comprising certain target microorganisms, and/or biofilms comprising certain target microorganisms. In various embodiments these kits typically comprise a chimeric moiety comprising a targeting peptide and a detectable label as described herein and/or a targeting peptide attached to an affinity tag for use in a pretargeting strategy as described herein.

In addition, the kits optionally include labeling and/or instructional materials providing directions (i.e., protocols) for the practice of the methods or use of the “therapeutics” or “prophylactics” or detection reagents of this invention. Certain instructional materials describe the use of one or more active agent(s) of this invention to therapeutically or prophylactically to inhibit or prevent C. difficile infection. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Synthesis and Evaluation of STAMPs Directed Against Clostridium difficile Methods

STAMP Design.

Several families of STAMPS shown in Table 7 directed against Clostridium difficile were chemically synthesized.

TABLE 7
Illustrative anti-C. difficile STAMPs. Linker is underlined.

| | Amino Acid Sequence | SEQ ID |
|---|---|---|
| STAMP | (Linker underlined if present) | NO |
| CD0714 + AF5_2G | VPAKLLRVIDEIPGGFLKFLKKFFKKLKY | 292 |
| CD0714 + AF5_2G_M4 | VPAKLLRVIKKIPGGFLKFLKKFFKKLK | 293 |
| CD0714 + AF5_2G_M7 | VPAKLLRVIKEIPGGFLKFLKKFFKKLK | 294 |
| CD0714 + BD2-21_NG | VPAKLLRVIDEIP KLFKFLRKHLL | 295 |
| CD0714 + Lys_A1_2G | VPAKLLRVIDEIPGGKIFGAIWPLALGALKNLIK | 296 |
| CD0126 + AF5_4G | LATKLKYEKEHKKMGGGGFLKFLKKFFKKLKYY | 297 |
| CD0126 + Lys_A1_1G | LATKLKYEKEHKKMGKIFGAIWPLALGALKNLIK | 298 |
| CD0126 + Lys_A1_M3 | LATLKKYLKEHKKMGKIFGAIWPLALGALKNLIK | 299 |
| CD9232_G2_3G | NILRVLKQVWKGGGKNLRIIRKGIHIIKKY | 300 |
| CD8040-G2_3G | GNFYRLFKDILKGGGKNLRIIRKGIHIIKKY | 301 |

The CD0714 family STAMPs (CD0714+AF5_2G, CD0714+AF5_2G_M4, CD0714+BD2-21_NG, and CD0714+Lys_A1_2G) were constructed by modifying the C. difficile binding sequence VPAKLLRVIDEIPE (PF-285, SEQ ID NO:2) by deletion of the terminal “E” to produce the targeting peptide VPAKLLRVIDEIP (SEQ ID No:3) (and further optimized in some embodiments (e.g., CD0714-AF52GM4), by replacing “DE” with “KK” to produce the targeting peptide VPAKLLRVIKKIP (SEQ ID NO:4).

For proof of principle, these sequences were combined with the killing (antimicrobial) peptides AF5 (FLKFLKKFFKKLKY, (SEQ ID NO:181)) and BD2-21 (KLFKFLRKHLL, (SEQ ID NO:134)) to yield a STAMP family. For evaluation purposes all the peptides had an amidated C-terminus. In CD0714-AF52GM4 (see Table 7), the C-terminal “Y” in killing peptide AF5 was also deleted.

The CD0126 family (CD0126+AF5_4G, CD0126+Lys_A1_1G) STAMPs were constructed by selecting CD0126 (SEQ ID NO:11), also known as PF-299). This sequence was combined with the killing peptide AF5 (modified with an extra terminal “Y”) to yield the following STAMP comprising a single amino acid linker “G” (CD0126+Lys_A1_1G) and a 4 amino acid linker GGGG (SEQ ID NO:265) (CD0126+AF5_4G). For evaluation purposes all the peptides had an amidated C-terminus.

Additional STAMPs (CD9232_G2_3G, CD8040-G2_3G) were constructed by combining several genomic peptides sequences identified from a recently sequenced and annotated C. difficile genome. They were combined with the killing peptide G2 (KNLRIIRKGIHIIKKY, (SEQ ID NO:169)).

Bacterial Growth.

C. difficile isolates ATCC 630 (strain 1382), ATCC BAA-1803, ATCC BAA-1875 (strain 5325), ATCC 43255 (strain 10463), and ATCC 43601 (strain 7322, non-toxigenic), and commensals B. fragilis (ATCC 2528), were grown in oxygen-reduced 1×BBL Brain-Heart Infusion (BD, Franklin Lakes, N.J.) supplemented with yeast and filter-sterilized 1% (w/v) L-cysteine (BHI-S). L. casei were grown in Difco Lactobaciili MRS (BD, Franklin, N.J.). All bacteria strains were cultivated at anaerobic conditions (10% H2, 10% CO2, 80% N₂) at 37° C. overnight and refresh to an OD₆₀₀ of 0.8-1.2 prior antimicrobial testing.

Peptide Synthesis and Purification.

Peptides were synthesized using standard solid-phase (Fmoc) chemistry with an Apex 396 peptide synthesizer (Aapptec, Louisville, Ky.) at a 0.01 mM scale. N-terminal deblocking was conducted with 0.6 ml of 25% (vol/vol) piperidine in dimethylformamide (DMF), followed by agitation for 27 min and wash cycles with dichlormethane (DCM) (1 ml; one wash cycle) and N-methylpyrrolidone (NMP) (0.8 ml; seven wash cycles). Subsequent amino acid coupling cycles were conducted with a mixture of Fmoc-protected amino acid (5 eq), HOBT (5 eq), HBTU (5 eq), N,N-diisopropylethylamine (DIEA; 10 eq)-DMF (0.1 ml), and NMP (0.2 ml) with agitation for 45 min. The washing cycle was repeated before the next round of deprotection and coupling. After synthesis, peptides were washed in methanol and dried for 24 h. Protected peptides were cleaved with 1 ml of trifluoroacetic acid (TFA)-thioanisole-water-1,2-ethanedithiol (10 ml:0.5 ml:0.5 ml:0.25 ml) mixture for 3 h at room temperature and the resultant peptide solution was precipitated in methyl tert-butyl ether.

Analytical and preparative HPLC was conducted as described previously (Eckert et al. (2006) Antimicrob. Agents Chemother. 50: 3833-3838; He et al. (2009) Int. J. Antimicrob. Agents, 33: 532-537) to refine each peptide to 80 to 90% purity. Correct peptide mass values were confirmed by matrix-assisted laser desorption ionization (MALDI) (Voyager 4219 workstation; Applied Biosystems, Foster City, Calif.) or electrospray ionization (ESI) mass spectroscopy (Waters 3100 mass detector; Waters, Milford, Mass.) as described previously (He et al. (2009) Int. J. Antimicrob. Agents, 33: 532-537). Measurements were made in linear, positive ion mode with an α-cyano-4-hydroxycinnamic acid matrix where appropriate (data not shown).

MICs of Peptides.

Peptide MICs were determined by broth microdilution (Eckert et al. (2006) Antimicrob. Agents Chemother. 50: 3651-3657; Qi, et al. (2005) FEMS Microbiol. Lett. 251: 321-326). Briefly, 2-fold serial dilutions of each peptide were prepared with 1× Mueller-Hinton broth) at a volume of 50 μl per well in 96-well flat-bottom microtiter plates. The concentrations of peptides ranged from 250 to 0.5 μg/ml. The microtiter plate was then inoculated with 150 μl of bacterial cell suspension per well at a final concentration of ˜2.5×10⁵ CFU/ml and incubated at 37° C. for 24 to 48 h under the appropriate conditions. After incubation, absorbance at 600 nm (A₆₀₀) was measured using a microplate UV-Vis spectrophotometer (model 3550; Bio-Rad, Hercules, Calif.) to assess cell growth. The MIC endpoint was calculated as the lowest concentration of antibacterial agent that completely inhibited growth or that produced an at least 90% reduction in turbidity compared with that of a peptide-free control. At least 3 independent tests were conducted per peptide.

Peptide Killing Kinetics.

Killing kinetics of each peptide were determined by CFU/plating method in planktonic liquid cultures. 2× concentration of each peptide and bacteria co-culture were prepared separately with BHI-S at a volume of 500 μL in 1.5 mL microcentrifuge tubes (USA Scientific, Ocala, Fla.). Peptides and bacteria were then mix together to reach a final concentration of ˜4×10⁵ CFU/mL of bacteria with appropriate peptide concentration. Cell viability was monitor at 5 different time points (1 MIN, 15 MIN, 30 MIN, 1 HR, and 2 HR) by CFU plating methods. Briefly, 50 μL of bacteria and peptide mixture were taken out at each time points and rescued onto 96-well flat-bottom micro titer polystyrene plates (Costar 3595, Corning, Corning, N.Y.). The bacterial culture was then diluted 5-fold, 4 times. Dilutions 3 and 4 were plated twice onto a reduced two-division sterile polystyrene petri plates 100×15 mm (VWR, Radnor, Pa.). Replicates of each dilution were performed onto each division. Petri plates were prepared with BHI-S and agar supplemented with a selective antibiotic for desired bacteria. The selective antibiotic and its concentration was determined through MIC screening against C. difficile and each desired commensal. The chosen antibiotics met the criteria of complete inhibition of one species and not affecting the growth of second species in mixture at the minimum concentration (data not shown) as follows: Erythromycin (1 μg/ml) plates were used to select C. difficile 1803 growth while vancomycin (1 μg/ml) plates were used to select L. casei growth for co-culture kinetic assays. Rifampcin (0.5 μg/ml) plates were used to select C. difficile 1803 growth and Metronidazole (1 μg/ml) plates were used to select B. fragilis for co-culture kinetic assay. Plates were incubated for ˜16-24 HRs to ensuring colonies were visible and accurately countable by automated colony counter (Protocol3, Synbiosis, Frederick, Md.).

### Results.

MIC Values.

MIC values for the STAMPs described above against C. difficile isolates, B. fragilis, and L. casei are shown in Table 8. As shown therein, STAMPs designed against C. difficile displayed MIC values of 62.5 μg/ml or below against a variety of C. difficile isolates tested. The peptides had relatively higher MIC values against the B. fragilis and L. casei isolates examined.

TABLE 8
MIC values (μg/ml) after 24 hrs

| | C. | C. | C. | C. | C. | | |
|---|---|---|---|---|---|---|---|
| | difficile | difficile | difficile | difficile | difficile | | |
| | isolate | isolate | isolate | isolate | isolate | B. | L. |
| Peptide | CD1382 | CD1803 | CD1875 | CD43255 | CD43601 | fragilis | casei |
| CD0714 + AF5_2G | 31.3 | 62.5 | 62.5 | 31.3 | N/A | >250 | >250 |
| CD0714 + AF5_2G_M4 | 3.9 | 7.8 | 15.6 | 7.8 | 3.9 | 62.5 | 125 |
| CD0714 + AF5_2G_M7 | 7.8 | 3.9 | 15.6 | 3.9 | 7.8 | >250 | >250 |
| CD0714 + BD2-21_NG | 7.8 | 15.6 | 15.6 | 7.8 | 3.9 | >250 | 125 |
| CD0714 + Lys_A1_2G | 31.3 | 125 | 31.3 | 62.5 | 15.6 | >250 | >250 |
| CD0126 + AF5_4G | 15.6 | 31.3 | 15.6 | 15.6 | 7.8 | >250 | >250 |
| CD0126 + Lys_A1_1G | 7.8 | 7.8 | 15.6 | 15.6 | 15.6 | >250 | >250 |
| CD0126 + Lys_A1_M3 | 3.9 | 7.8 | 7.8 | 7.8 | 7.8 | >250 | >250 |
| CD9232_G2_3G | 3.9 | 7.8 | 31.3 | 7.8 | 7.8 | 15.6 | 125 |
| CD8040_G2_3G | 15.6 | 15.6 | 62.5 | 31.3 | 31.3 | 62.5 | >250 |

STAMP Activity.

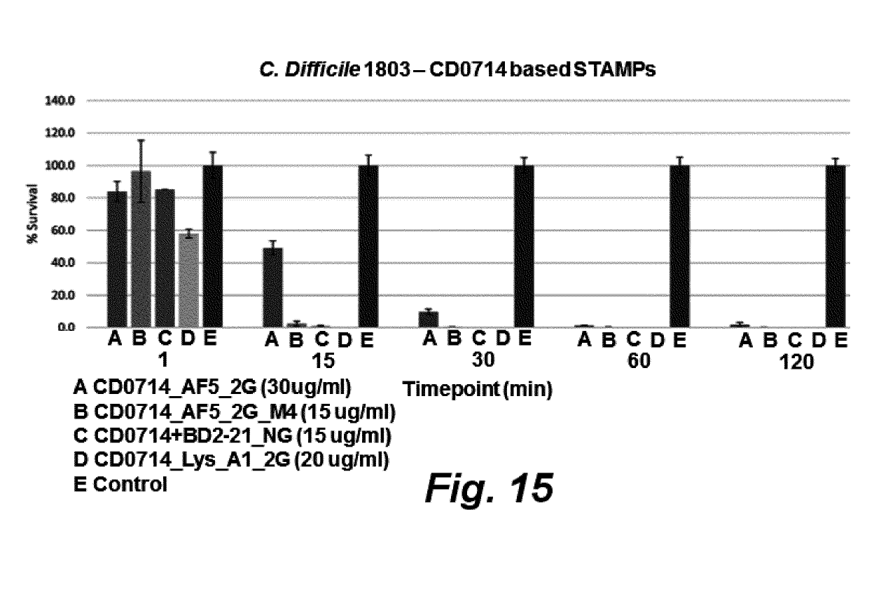

FIG. 15 shows the killing kinetics of CD0714-based STAMPs on C. difficile isolate 1803. The data indicate that >90% killing of C. difficile is obtained by all the STAMPs examined by 30 minutes, suggesting a rapid killing mechanism and activity.

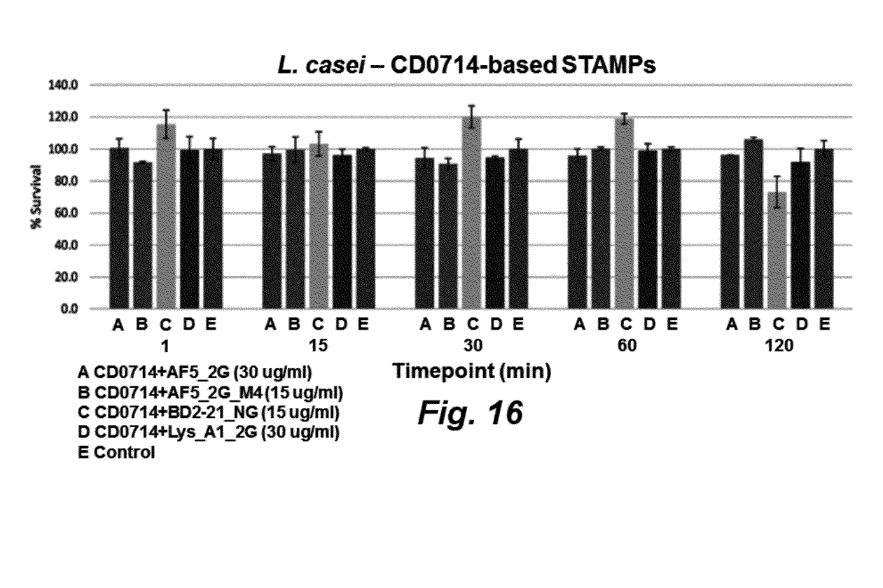

FIG. 16 shows the killing kinetics of CD0714-based STAMPs on L. casei. Against the un-targeted organism L. casei the STAMPs examined were less active compared to the levels seen in FIG. 15. Surviving L. casei did not significantly decrease through 2 hours of STAMP exposure.

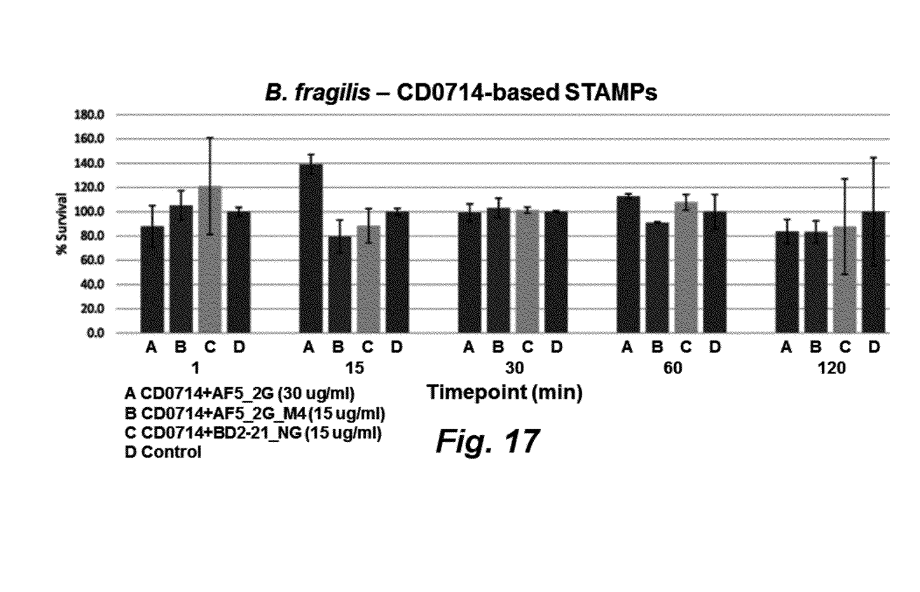

FIG. 17 shows the killing kinetics of CD0714-based STAMPs on B. fragilis. Against the un-targeted organism B. fragilis the STAMPs examined were less active compared to the levels seen in FIG. 15. Surviving B. fragilis did not significantly decrease through 2 hours of STAMP exposure.

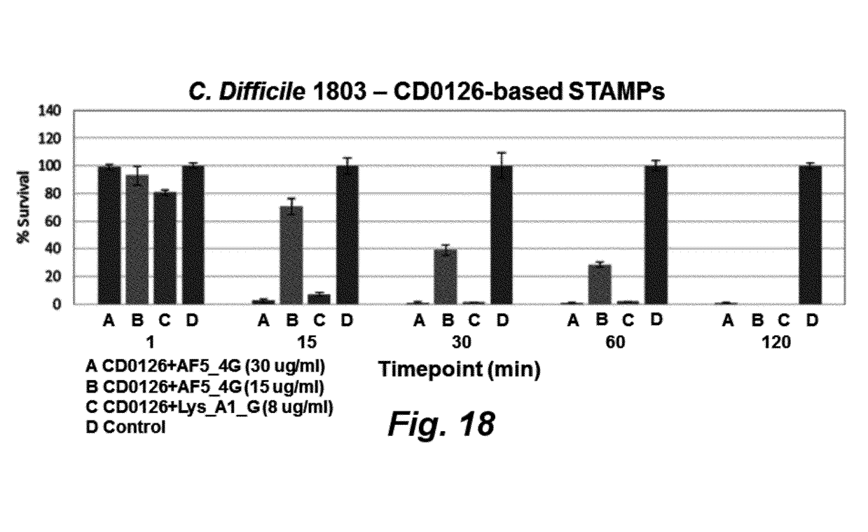

FIG. 18 shows the killing kinetics of CD0126-based STAMPs on C. difficile isolate 1803. The data indicate that >90% killing of C. difficile is obtained by all the STAMPs examined by 120 minutes, suggesting a rapid killing mechanism and activity. STAMPs CD0126+AF5_4G and CD0126+Lys_A1_1G reduced the viable C. difficile recovered to <90% by 15 minutes, suggesting robust activity.

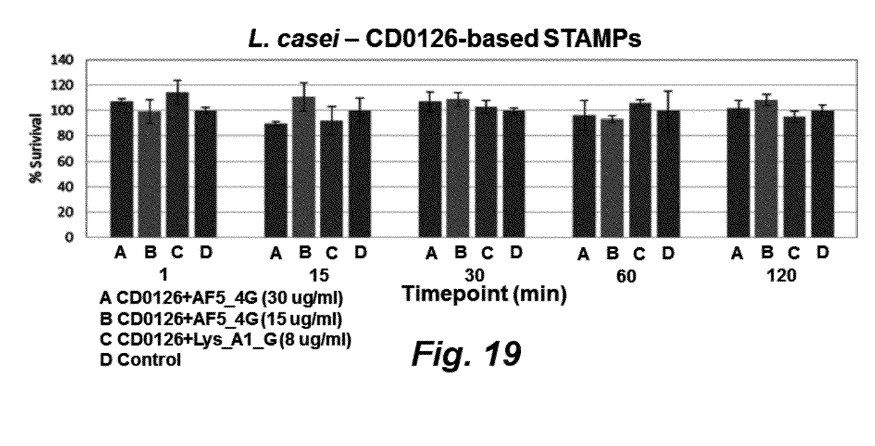

FIG. 19 shows the killing kinetics of CD0126-based STAMPs on L. casei. Against the un-targeted organism L. casei the STAMPs examined were less active compared to the levels seen in FIG. 18. Surviving L. casei did not significantly decrease through 2 hours of STAMP exposure.

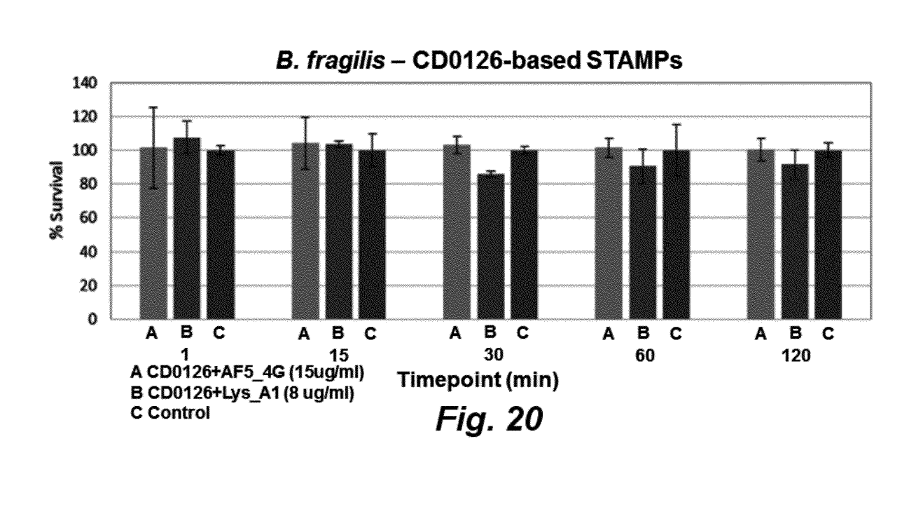

FIG. 20 shows the killing kinetics of STAMP CD0126+AF5_4G on B. fragilis. Against the un-targeted organism B. fragilis the STAMPs examined were less active compared to the levels seen in FIG. 18. Surviving B. fragilis did not significantly decrease through 2 hours of STAMP exposure.

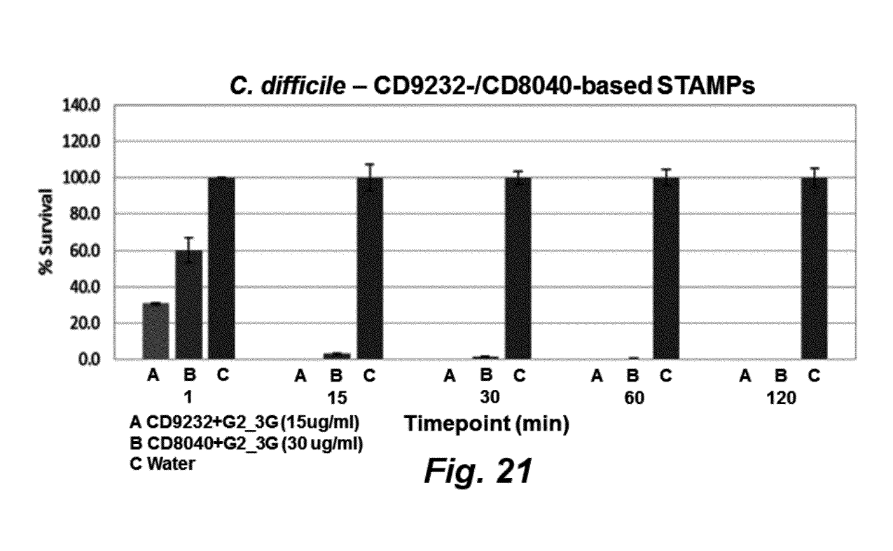

FIG. 21 shows the killing kinetics of CD9232/CD8040-based STAMPs on C. difficile isolate 1803. The data indicate that >90% killing of C. difficile is obtained by all the STAMPs examined by 120 minutes, suggesting a rapid killing mechanism and activity. STAMPs CD9232+G2_3G and CD8040+G2_3G reduced the viable C. difficile recovered to <90% by 15 minutes, suggesting robust activity.

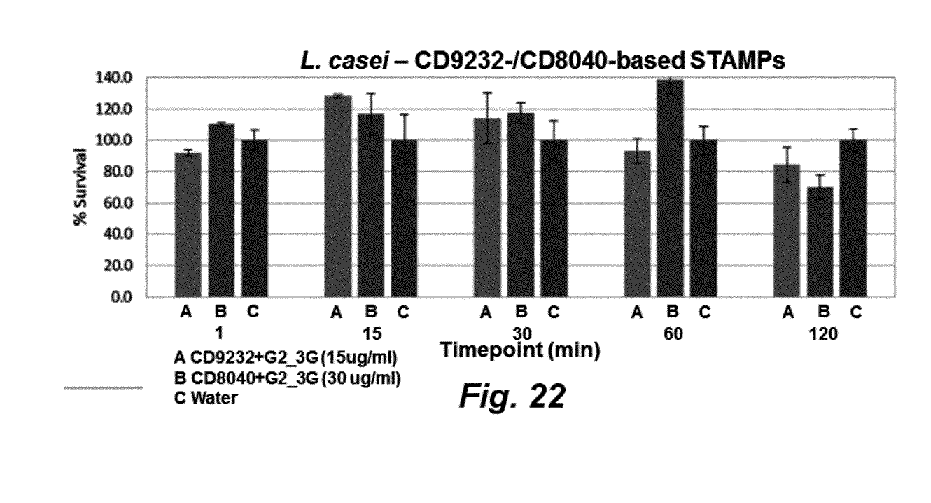

FIG. 22 shows the killing kinetics of CD9232/CD8040-based STAMPs on L. casei. Against the un-targeted organism L. casei the STAMPs examined were less active compared to the levels seen in FIG. 21. Surviving L. casei did not significantly decrease through 2 hours of STAMP exposure.

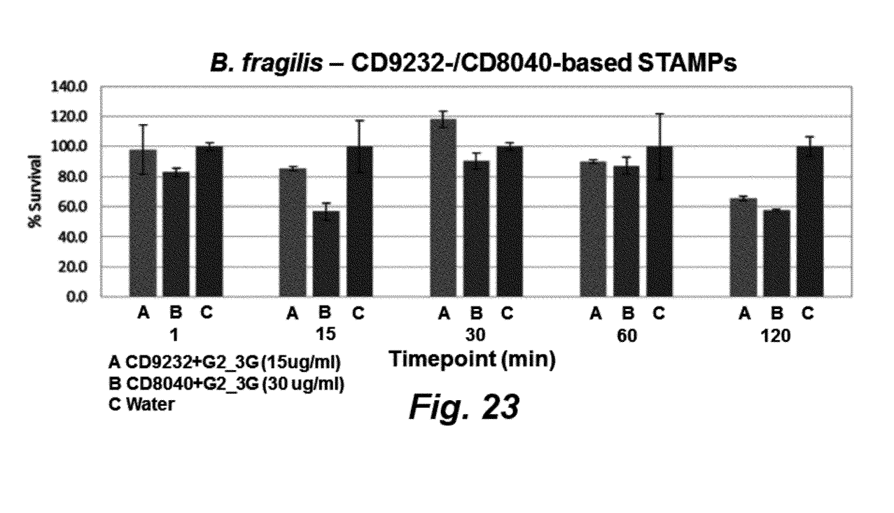

FIG. 23 shows the killing kinetics of CD9232/CD8040-based STAMPs on B. fragilis. Against the un-targeted organism B. fragilis STAMPs CD9232+G2_3G and CD8040+G2_3G were less active compared to the levels seen in FIG. 21. Surviving B. fragilis did not significantly decrease through 2 hours of STAMP exposure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A targeting peptide that binds to Clostridium difficile, where said peptide comprises or consists of an amino acid sequence selected from the group consisting of:
VPAKLLRVIDEIP (SEQ ID NO:3) where said peptide does not comprise the amino acid sequence VPAKLLRVIDEIPE (SEQ ID NO:2);
VPAKLLRVIKKIP (SEQ ID NO:4), VPAKLLRVIKEIP (SEQ ID NO:5), NILRVLKQVWK (SEQ ID NO:20), GNFYRLFKDILK (SEQ ID NO:28);
a fragment of VPAKLLRVIDEIP (SEQ ID NO:3) comprising at least 6, or at least 8, or at least 10 contiguous residues of said sequence where said peptide does not comprise the amino acid sequence VPAKLLRVIDEIPE (SEQ ID NO:2);
a fragment of VPAKLLRVIKKIP (SEQ ID NO:4) comprising at least 6, or at least 8, or at least 10 contiguous residues of said sequence;
a fragment of NILRVLKQVWK (SEQ ID NO:20) comprising at comprising at least 6, or at least 8, or at least 10 contiguous residues of said sequence;
a fragment of GNFYRLFKDILK (SEQ ID NO:28) comprising at least 6, or at least 8, or at least 10 contiguous residues of said sequence;
an inverso form of any of the preceding amino acid sequences; and
an amino acid sequence that has at least 90% sequence identity with an amino acid sequence selected from the group consisting of VPAKLLRVIDEIP (SEQ ID NO:3), VPAKLLRVIKKIP (SEQ ID NO:4), VPAKLLRVIKEIP (SEQ ID NO:5), NILRVLKQVWK (SEQ ID NO:20), and GNFYRLFKDILK (SEQ ID NO:28), and where said sequence does not contain the amino acid sequence VPAKLLRVIDEIPE (SEQ ID NO:2);
wherein said targeting peptide binds to C. difficile.

2-12. (canceled)

13. The targeting peptide of claim 1, wherein said peptide is attached to an antimicrobial peptide.

14. A construct comprising:
a targeting peptide of claim 1, or a targeting peptide comprising or consisting of the amino acid sequence LATKLKYEKEHKKM (SEQ ID NO:11) or that comprises or consists of the amino acid sequence LATLKKYLKEHKKM (SEQ ID NO:12), where said targeting peptide is attached to an effector moiety selected from the group consisting of a detectable label, a porphyrin or other photosensitizer, an antimicrobial peptide, an antibiotic, a ligand, a lipid or liposome, an agent that physically disrupts the extracellular matrix within a community of microorganisms, and a polymeric particle.

15. The construct of claim 14, wherein said targeting peptide is attached to an antimicrobial peptide comprising or consisting of an amino acid sequence found in Table 3.

16. The construct of claim 14, wherein said targeting peptide is attached to an antimicrobial peptide comprising or consisting of an amino acid sequence selected from the group consisting of KNLRIIRKGIHIIKKY ((SEQ ID NO:169, G2), FLKFLKKFFKKLKYY (SEQ ID NO:42), KNLRRIIRKGIHIIKKYG (SEQ ID NO:197), Novispirin G10), KNLRRIIRKTIHIIKKYG (SEQ ID NO:198, Novispirin T10), KNLRRIGRKIIHIIKKYG (SEQ ID NO:199, Novispirin G7), KNLRRITRKIIHIIKKYG (SEQ ID NO:200, Novispirin T7), KNLRRIIRKIIHIIKKYG (SEQ ID NO:201, Ovispirin), PGGGLLRRLRKKIGEIFKKYG (SEQ ID NO:302), RGGRLCYCRRRFCVCVGR (SEQ ID NO:234, Protegrin-1), GLGRVIGRLIKQIIWRR (SEQ ID NO:170, K-1), VYRKRKSILKIYAKLKGWH (SEQ ID NO:180, K-2), NYRLVNAIFSKIFKKKFIKF (SEQ ID NO:184, K-7), KILKFLFKKVF (SEQ ID NO:185, K-8), FIRKFLKKWLL (SEQ ID NO:186, K-9), KLFKFLRKHLL (SEQ ID NO:134, K-10), KILKFLFKQVF (SEQ ID NO:171, K-11), KILKKLFKFVF (SEQ ID NO:172, K-12), GILKKLFTKVF (SEQ ID NO:173, K-13), LRKFLHKLF (SEQ ID NO:174, K-14), LRKNLRWLF (SEQ ID NO:175, K-15), FIRKFLQKLHL (SEQ ID NO:176, K-16), FTRKFLKFLHL (SEQ ID NO:177, K-17), KKFKKFKVLKIL (SEQ ID NO:178, K-18), LLKLLKLKKLKF (SEQ ID NO:179, K-19), FLKFLKKFFKKLKY (SEQ ID NO:181, K-20), GWLKMFKKIIGKFGKF (SEQ ID NO:182, K-21), GIFKKFVKILYKVQKL (SEQ ID NO: 183, K-22), FKKFWKWFRRF (SEQ ID NO:90, B-33), FELVDWLETNLGKILKSKSA (SEQ ID NO:218, PF-522), and YIQFHLNQQPRPKVKKIKIFL (SEQ ID NO: 225, PF-531).

17. The construct of claim 14, wherein said targeting peptide and said antimicrobial peptide are “L” peptides.

18. The construct of claim 14, wherein the carboxyl terminal residue of said construct or the amino terminal residue of said construct is a “D” amino acid.

19. (canceled)

20. The construct of claim 14, wherein said targeting peptide and said antimicrobial peptide are “D” peptides or are beta peptides.

21. (canceled)

22. The construct of claim 14, wherein said targeting peptide is chemically conjugated to said effector.

23-25. (canceled)

26. The construct of claim 14, wherein said targeting peptide is linked directly to said effector.

27. The construct of claim 14, wherein said targeting peptide is linked to said effector via a single amino acid or via a peptide linkage.

28. The construct of claim 27, wherein said effector comprises an antimicrobial peptide and said construct is a fusion protein.

29. The construct of claim 27, wherein said targeting peptide is attached to said effector by a single amino acid or by a peptide linker comprising or consisting of an amino acid sequence found in Table 6.

30. (canceled)

31. The construct of claim 14, wherein the amino acid sequence of said construct comprises or consists of an amino acid sequence selected from the group consisting of VPAKLLRVIDEIPGGFLKFLKKFFKKLKY (SEQ ID NO:293, CD0714+AF5_2G), VPAKLLRVIKKIPGGFLKFLKKFFKKLK (SEQ ID NO:294, CD0714+AF5_2G_M4), VPAKLLRVIKEIPGGFLKFLKKFFKKLK (SEQ ID NO:295, CD0714+AF5_2G_M7), VPAKLLRVIDEIPKLFKFLRKHLL (SEQ ID NO:296, CD0714+BD2-21_NG), VPAKLLRVIDEIPGGKIFGAIWPLALGALKNLIK (SEQ ID NO:297, CD0714+Lys_A1_2G), LATKLKYEKEHKKMGGGGFLKFLKKFFKKLKYY (SEQ ID NO:298, CD0126+AF5_4G), LATKLKYEKEHKKMGKIFGAIWPLALGALKNLIK (SEQ ID NO:299, CD0126+Lys_A1_1G), LATLKKYLKEHKKMGKIFGAIWPLALGALKNLIK (SEQ ID NO:300, CD0126+Lys_A1_M3), NILRVLKQVWKGGGKNLRIIRKGIHIIKKY (SEQ ID NO:301, CD9232_G2_3G), and GNFYRLFKDILKGGGKNLRIIRKGIHIIKKY (SEQ ID NO:302, CD8040-G2_3G).

32. The construct of claim 14, wherein said construct further comprises a chemoattractant peptide sequence.

33-39. (canceled)

40. The construct of claim 14, wherein said construct bears no terminal protecting groups.

41. The construct of claim 14, wherein said construct bears one or more protecting groups.

42-47. (canceled)

48. An antimicrobial peptide (AMP) comprising or consisting of the amino acid sequence FLKFLKKFFKKLKYY (SEQ ID NO:42) or a truncated version thereof that retains antimicrobial activity.

49-54. (canceled)

55. A pharmaceutical composition comprising a construct of claim 14, in a pharmaceutically acceptable carrier.

56-57. (canceled)

58. The pharmaceutical composition of claim 55, wherein said composition is formulated for specific or preferential delivery to the colon, esophagus, stomach, large intestine, or small intestine in a mammal.

59. The pharmaceutical composition of claim 58, wherein said composition is formulated for specific or preferential delivery to the colon in a mammal using a formulation form selected from the group consisting of pH sensitive coating, delayed time-controlled release system, microbially triggered drug delivery, pressure controlled drug delivery, Colon Targeted Delivery System (CODES™), and Osmotic Controlled Drug Delivery (ORDS-CT).

60-76. (canceled)

77. A method of killing or inhibiting the growth or proliferation of Clostridium difficile, said method comprising:
contacting said C. difficile with a composition comprising a construct of claim 14, in an amount effective to kill or inhibit the growth or proliferation of C. difficile.

78-83. (canceled)

84. A composition comprising fecal matter for fecal transplantation, said composition comprising fecal matter combined with a construct of claim 1.

85-88. (canceled)

89. A method of preparing material for fecal transplantation, said method comprising combining material comprising stool from a mammal with a construct of claim 14, in an amount sufficient to preferentially reduce or eliminate viable C. difficile in said stool.

90. A method of treating a gasteroenterologic disease, in a subject in need thereof, said method comprising performing a fecal matter transplant into said subject using a composition of claim 84.

91-106. (canceled)
